SEQUENCE LISTING (8 sequences)

SEQ ID NO: 1 - protein, 6 aa, Rattus sp.
ESESRG

SEQ ID NO: 2 - protein, 6 aa, Rattus sp.
ESENVY

SEQ ID NO: 3 - protein, 883 aa, Rattus sp.
MIPLLLSLLAALVLTQAPAALADDLKEDSSEDRAFRVRIGAAQLRGVLGGALAIPCHVHHLRPPPSRRAAPGFPRVKWTFLSGDREVEVLVARGLRVKVNEAYRFRVALPAYPASLTDVSLVLSELRPNDSGVYRCEVQHGIDDSSDAVEVKVKGVVFLYREGSARYAFSFAGAQEACARIGARIATPEQLYAAYLGGYEQCDAGWLSDQTVRYPIQNPREACYGDMDGYPGVRNYGVVGPDDLYDVYCYAEDLNGELFLGAPPGKLTWEEARDYCLERGAQIASTGQLYAAWNGGLDRCSPGWLADGSVRYPIITPSQRCGGGLPGVKTLFLFPNQTGFPSKQNRFNVYCFRDSAHPSAFSEASSPASDGLEAIVTVTEKLEELQLPQEAVESENVYAIYSIPITEDGGGGSSTPEDPAEAPRTPLESETQSVAPPTGSSEEEGEALEEEERFKDTETPKEEKEQENLWVWPTELSSPLPTGLETEHSLSQVSPPAQAVLQLGASPSPRPPRVHGPPAETLQPPREGSLTSTPDGAREVAGETGSPELSGVPRESEEAGSSSLEDGPSLLPATWAPVGTRELETPSEEKSGRTVLTGTSVQAQPVLPTDSASRGGVAVAPSSGDCIPSPCHNGGTCLEEKEGFRCLCLPGYGGDLCDVGLHFCSPGWEAFQGACYKHFSTRRSWEEAESQCRALGAHLTSICTPEEQDFVNDRYREYQWIGLNDRTIEGDFLWSDGAPLLYENWNPGQPDSYFLSGENCVVMVWHDQGQWSDVPCNYHLSYTCKMGLVSCGPPPQLPLAQIFGRPRLAYAVDTVLRYRCRDGLAQRNLPLIRCQENGLWEAPQISCVPRRPARALRSMTAPEGPRGQLPRQRKALLTPPSSL

SEQ ID NO: 4 - DNA, 2652 bp, Rattus sp.
atgatcccattgcttctgtccctgctggcagctctggtcctgacccaagcccctgcagccctcgctgatgacctgaaagaagacagctcagaggatcgagcctttcgggtgcgcatcggtgccgcgcagctgcggggtgtgctgggcggtgccctggccatcccatgccacgtccaccacctgaggccgccgcccagccgccgggccgcgccgggctttccccgagtcaaatggaccttcctgtccggggaccgggaggtggaggtgctggtggcgcgcgggctgcgcgtcaaggtaaacgaagcctatcggttccgcgtggcgctgcctgcctaccccgcatcgctcacagatgtgtctttagtattgagcgaactgcggcccaatgattccggggtctatcgctgcgaggtccagcacggtatcgacgacagcagtgatgctgtggaagtcaaggtcaaaggggtcgtcttcctctaccgagagggctctgcccgctatgctttctccttcgctggagcccaggaagcctgtgctcgcatcggagcccgaattgccacccctgagcagctgtatgctgcctacctcggcggctatgaacagtgtgatgctggctggctgtccgaccaaaccgtgaggtaccccatccagaacccacgagaagcctgttatggagacatggatggctaccctggagtgcggaattacggagtggtgggtcctgatgatctctacgatgtctactgttatgccgaagacctaaatggagaactgttcctaggtgcccctcccggcaagctgacgtgggaggaggctcgggactactgtctggaacgcggtgctcagatcgctagcacgggccagctatacgcggcatggaatggcggcttggacagatgtagccctggctggctggctgatggcagtgtgcggtaccccatcatcacgcccagccaacgctgtgggggaggcctgccaggagtcaagaccctcttcctctttcccaaccagactggcttccccagcaagcagaaccgcttcaatgtctactgcttccgagactctgcccatccctctgccttctctgaggcctccagcccagcctctgatggactagaggccattgtcacagtgacagagaagctggaggaactgcagttgcctcaggaagctgtggagagcgagaatgtttacgcgatctactccatccccatcacagaagatgggggaggaggaagctctaccccagaagacccagcagaggcccccaggactcctctagaatcagaaacccaatccgttgcaccacctaccgggtcctcagaagaggaaggcgaagccctggaggaagaagaaagattcaaagacacagagactccgaaggaagagaaggagcaggagaacctgtgggtgtggcccacggagctcagcagccctctccctactggcttggaaacagagcactcactctcccaggtgtccccaccagcccaggcagttctacagctgggtgcatcaccttctcccaggcctccaagggtccatggaccgcctgcagagactttgcaacccccaagggagggaagcctcacatctactccagatggggcaagagaagtagcgggggaaactgggagccctgagctctctggggttcctcgagaaagcgaggaggcaggaagctccagcttggaggatggcccttccctccttccagcgacatgggcccctgtgggtaccagggagctggagaccccctcagaagagaagtctggaagaactgttctgacaggcacatcagtgcaggcccagccagtgctgcccaccgacagtgccagccgaggtggagtggctgtggctccctcatcaggtgactgtatccccagcccctgccacaatggtgggacatgcttggaggagaaggagggtttccgctgcctctgtttgccaggctatgggggggacctgtgcgatgttggcctccacttctgcagcccgggctgggaggccttccagggtgcctgctacaagcacttttccacacgaaggagttgggaggaggcagaaagccagtgccgagcgctaggggctcatctgaccagcatctgcacccctgaggagcaggactttgtcaacgatcgatacagggagtaccagtggattgggctcaatgacaggaccatcgagggtgacttcctgtggtcagatggtgcccctctgctctatgaaaactggaaccctgggcagcctgacagctacttcctgtctggggagaactgtgtggtcatggtgtggcatgaccagggacagtggagtgatgtaccctgcaactaccacctatcctacacctgcaagatggggcttgtgtcatgtggacctccaccacagctgcccctggctcaaatatttggtcgccctcggctggcctacgcggtggacactgtgcttcgatatcggtgccgagacgggctggcccagcgcaacttgccgttgatccgctgccaggagaatgggctttgggaggcccctcagatttcttgcgtgccccgaagacctgcccgtgctctccgctcaatgaccgccccagaaggaccacggggacagctcccgaggcagaggaaagcactgttgacacctccctccagtctctag

SEQ ID NO: 5 - DNA, 2652 bp, Rattus sp.
atgatcccattgcttctgtccctgctggcagctctggtcctgacccaagcccctgcagccctcgctgatgacctgaaagaagacagctcagaggatcgagcctttcgggtgcgcatcggtgccgcgcagctgcggggtgtgctgggcggtgccctggccatcccatgccacgtccaccacctgaggccgccgcccagccgccgggccgcgccgggctttccccgagtcaaatggaccttcctgtccggggaccgggaggtggaggtgctggtggcgcgcgggctgcgcgtcaaggtaaacgaagcctatcggttccgcgtggcgctgcctgcctaccccgcatcgctcacagatgtgtctttagtattgagcgaactgcggcccaatgattccggggtctatcgctgcgaggtccagcacggtatcgacgacagcagtgatgctgtggaagtcaaggtcaaaggggtcgtcttcctctaccgagagggctctgcccgctatgctttctccttcgctggagcccaggaagcctgtgctcgcatcggagcccgaattgccacccctgagcagctgtatgctgcctacctcggcggctatgaacagtgtgatgctggctggctgtccgaccaaaccgtgaggtaccccatccagaacccacgagaagcctgttatggagacatggatggctaccctggagtgcggaattacggagtggtgggtcctgatgatctctacgatgtctactgttatgccgaagacctaaatggagaactgttcctaggtgcccctcccggcaagctgacgtgggaggaggctcgggactactgtctggaacgcggtgctcagatcgctagcacgggccagctatacgcggcatggaatggcggcttggacagatgtagccctggctggctggctgatggcagtgtgcggtaccccatcatcacgcccagccaacgctgtgggggaggcctgccaggagtcaagaccctcttcctctttcccaaccagactggcttccccagcaagcagaaccgcttcaatgtctactgcttccgagactctgcccatccctctgccttctctgaggcctccagcccagcctctgatggactagaggccattgtcacagtgacagagaagctggaggaactgcagttgcctcaggaagctgtggagagcgagtctcgtggggcgatctactccatccccatcacagaagatgggggaggaggaagctctaccccagaagacccagcagaggcccccaggactcctctagaatcagaaacccaatccgttgcaccacctaccgggtcctcagaagaggaaggcgaagccctggaggaagaagaaagattcaaagacacagagactccgaaggaagagaaggagcaggagaacctgtgggtgtggcccacggagctcagcagccctctccctactggcttggaaacagagcactcactctcccaggtgtccccaccagcccaggcagttctacagctgggtgcatcaccttctcccaggcctccaagggtccatggaccgcctgcagagactttgcaacccccaagggagggaagcctcacatctactccagatggggcaagagaagtagcgggggaaactgggagccctgagctctctggggttcctcgagaaagcgaggaggcaggaagctccagcttggaggatggcccttccctccttccagcgacatgggcccctgtgggtaccagggagctggagaccccctcagaagagaagtctggaagaactgttctgacaggcacatcagtgcaggcccagccagtgctgcccaccgacagtgccagccgaggtggagtggctgtggctccctcatcaggtgactgtatccccagcccctgccacaatggtgggacatgcttggaggagaaggagggtttccgctgcctctgtttgccaggctatgggggggacctgtgcgatgttggcctccacttctgcagcccgggctgggaggccttccagggtgcctgctacaagcacttttccacacgaaggagttgggaggaggcagaaagccagtgccgagcgctaggggctcatctgaccagcatctgcacccctgaggagcaggactttgtcaacgatcgatacagggagtaccagtggattgggctcaatgacaggaccatcgagggtgacttcctgtggtcagatggtgcccctctgctctatgaaaactggaaccctgggcagcctgacagctacttcctgtctggggagaactgtgtggtcatggtgtggcatgaccagggacagtggagtgatgtaccctgcaactaccacctatcctacacctgcaagatggggcttgtgtcatgtggacctccaccacagctgcccctggctcaaatatttggtcgccctcggctggcctacgcggtggacactgtgcttcgatatcggtgccgagacgggctggcccagcgcaacttgccgttgatccgctgccaggagaatgggctttgggaggcccctcagatttcttgcgtgccccgaagacctgcccgtgctctccgctcaatgaccgccccagaaggaccacggggacagctcccgaggcagaggaaagcactgttgacacctccctccagtctctag

SEQ ID NO: 6 - protein, 883 aa, Rattus sp.
MIPLLLSLLAALVLTQAPAALADDLKEDSSEDRAFRVRIGAAQLRGVLGGALAIPCHVHHLRPPPSRRAAPGFPRVKWTFLSGDREVEVLVARGLRVKVNEAYRFRVALPAYPASLTDVSLVLSELRPNDSGVYRCEVQHGIDDSSDAVEVKVKGVVFLYREGSARYAFSFAGAQEACARIGARIATPEQLYAAYLGGYEQCDAGWLSDQTVRYPIQNPREACYGDMDGYPGVRNYGVVGPDDLYDVYCYAEDLNGELFLGAPPGKLTWEEARDYCLERGAQIASTGQLYAAWNGGLDRCSPGWLADGSVRYPIITPSQRCGGGLPGVKTLFLFPNQTGFPSKQNRFNVYCFRDSAHPSAFSEASSPASDGLEAIVTVTEKLEELQLPQEAVESESRGAIYSIPITEDGGGGSSTPEDPAEAPRTPLESETQSVAPPTGSSEEEGEALEEEERFKDTETPKEEKEQENLWVWPTELSSPLPTGLETEHSLSQVSPPAQAVLQLGASPSPRPPRVHGPPAETLQPPREGSLTSTPDGAREVAGETGSPELSGVPRESEEAGSSSLEDGPSLLPATWAPVGTRELETPSEEKSGRTVLTGTSVQAQPVLPTDSASRGGVAVAPSSGDCIPSPCHNGGTCLEEKEGFRCLCLPGYGGDLCDVGLHFCSPGWEAFQGACYKHFSTRRSWEEAESQCRALGAHLTSICTPEEQDFVNDRYREYQWIGLNDRTIEGDFLWSDGAPLLYENWNPGQPDSYFLSGENCVVMVWHDQGQWSDVPCNYHLSYTCKMGLVSCGPPPQLPLAQIFGRPRLAYAVDTVLRYRCRDGLAQRNLPLIRCQENGLWEAPQISCVPRRPARALRSMTAPEGPRGQLPRQRKALLTPPSSL

SEQ ID NO: 7 - DNA, 2878 bp, Homo sapiens
tgtggcactgcctgcgtacccaaccccagccctgggtagcctgcagcatggcccagctgttcctgcccctgctggcagccctggtcctggcccaggctcctgcagctttagcagatgttctggaaggagacagctcagaggaccgcgcttttcgcgtgcgcatcgcgggcgacgcgccactgcagggcgtgctcggcggcgccctcaccatcccttgccacgtccactacctgcggccaccgccgagccgccgggctgtgctgggctctccgcgggtcaagtggactttcctgtcccggggccgggaggcagaggtgctggtggcgcggggagtgcgcgtcaaggtgaacgaggcctaccggttccgcgtggcactgcctgcgtacccagcgtcgctcaccgacgtctccctggcgctgagcgagctgcgccccaacgactcaggtatctatcgctgtgaggtccagcacggcatcgatgacagcagcgacgctgtggaggtcaaggtcaaaggggtcgtctttctctaccgagagggctctgcccgctatgctttctccttttctggggcccaggaggcctgtgcccgcattggagcccacatcgccaccccggagcagctctatgccgcctaccttgggggctatgagcaatgtgatgctggctggctgtcggatcagaccgtgaggtatcccatccagaccccacgagaggcctgttacggagacatggatggcttccccggggtccggaactatggtgtggtggacccggatgacctctatgatgtgtactgttatgctgaagacctaaatggagaattgttcctgggtgaccctccagagaagctgacattggaggaagcacgggcgtactgccaggagcggggtgcagagattgccaccacgggccaactgtatgcagcctgggatggtggcctggaccactgcagcccagggtggctagctgatggcagtgtgcgctaccccatcgtcacacccagccagcgctgtggtgggggcttgcctggtgtcaagactctcttcctcttccccaaccagactggcttccccaataagcacagccgcttcaacgtctactgcttccgagactcggcccagccttctgccatccctgaggcctccaacccagcctccaacccagcctctgatggactagaggctatcgtcacagtgacagagaccctggaggaactgcagctgcctcaggaagccacagagagtgaatcccgtggggccatctactccatccccatcatggaggacggaggaggtggaagctccactccagaagacccagcagaggcccctaggacgctcctagaatttgaaacacaatccatggtaccgcccacggggttctcagaagaggaaggtaaggcattggaggaagaagagaaatatgaagatgaagaagagaaagaggaggaagaagaagaggaggaggtggaggatgaggctctgtgggcatggcccagcgagctcagcagcccgggccctgaggcctctctccccactgagccagcagcccaggaggagtcactctcccaggcgccagcaagggcagtcctgcagcctggtgcatcaccacttcctgatggagagtcagaagcttccaggcctccaagggtccatggaccacctactgagactctgcccactcccagggagaggaacctagcatccccatcaccttccactctggttgaggcaagagaggtgggggaggcaactggtggtcctgagctatctggggtccctcgaggagagagcgaggagacaggaagctccgagggtgccccttccctgcttccagccacacgggcccctgagggtaccagggagctggaggccccctctgaagataattctggaagaactgccccagcagggacctcagtgcaggcccagccagtgctgcccactgacagcgccagccgaggtggagtggccgtggtccccgcatcaggtgactgtgtccccagcccctgccacaatggtgggacatgcttggaggaggaggaaggggtccgctgcctatgtctgcctggctatgggggggacctgtgcgatgttggcctccgcttctgcaaccccggctgggacgccttccagggcgcctgctacaagcacttttccacacgaaggagctgggaggaggcagagacccagtgccggatgtacggcgcgcatctggccagcatcagcacacccgaggaacaggacttcatcaacaaccggtaccgggagtaccagtggatcggactcaacgacaggaccatcgaaggcgacttcttgtggtcggatggcgtccccctgctctatgagaactggaaccctgggcagcctgacagctacttcctgtctggagagaactgcgtggtcatggtgtggcatgatcagggacaatggagtgacgtgccctgcaactaccacctgtcctacacctgcaagatggggctggtgtcctgtgggccgccaccggagctgcccctggctcaagtgttcggccgcccacggctgcgctatgaggtggacactgtgcttcgctaccggtgccgggaaggactggcccagcgcaatctgccgctgatccgatgccaagagaacggtcgttgggaggccccccagatctcctgtgtgcccagaagacctgcccgagctctgcacccagaggaggacccagaaggacgtcaggggaggctactgggacgctggaaggcgctgttgatccccccttccagccccatgccaggtccctagggggcaaggccttgaacactgccggccacagcactgccctgtcacccaaattttccctcacaccctgcgctcaccacaggaagtgacaacatgac

SEQ ID NO: 8 - protein, 911 aa, Homo sapiens
MAQLFLPLLAALVLAQAPAALADVLEGDSSEDRAFRVRIAGDAPLQGVLGGALTIPCHVHYLRPPPSRRAVLGSPRVKWTFLSRGREAEVLVARGVRVKVNEAYRFRVALPAYPASLTDVSLALSELRPNDSGIYRCEVQHGIDDSSDAVEVKVKGVVFLYREGSARYAFSFSGAQEACARIGAHIATPEQLYAAYLGGYEQCDAGWLSDQTVRYPIQTPREACYGDMDGFPGVRNYGVVDPDDLYDVYCYAEDLNGELFLGDPPEKLTLEEARAYCQERGAEIATTGQLYAAWDGGLDHCSPGWLADGSVRYPIVTPSQRCGGGLPGVKTLFLFPNQTGFPNKHSRFNVYCFRDSAQPSAIPEASNPASNPASDGLEAIVTVTETLEELQLPQEATESESRGAIYSIPIMEDGGGGSSTPEDPAEAPRTLLEFETQSMVPPTGFSEEEGKALEEEEKYEDEEEKEEEEEEEEVEDEALWAWPSELSSPGPEASLPTEPAAQEESLSQAPARAVLQPGASPLPDGESEASRPPRVHGPPTETLPTPRERNLASPSPSTLVEAREVGEATGGPELSGVPRGESEETGSSEGAPSLLPATRAPEGTRELEAPSEDNSGRTAPAGTSVQAQPVLPTDSASRGGVAVVPASGDCVPSPCHNGGTCLEEEEGVRCLCLPGYGGDLCDVGLRFCNPGWDAFQGACYKHFSTRRSWEEAETQCRMYGAHLASISTPEEQDFINNRYREYQWIGLNDRTIEGDFLWSDGVPLLYENWNPGQPDSYFLSGENCVVMVWHDQGQWSDVPCNYHLSYTCKMGLVSCGPPPELPLAQVFGRPRLRYEVDTVLRYRCREGLAQRNLPLIRCQENGRWEAPQISCVPRRPARALHPEEDPEGRQGRLLGRWKALLIPPSSPMPGP

(19) United States
(12) Patent Application Publication
(10) Pub. No.: US 2003/0224976 A1
(43) Pub. Date: Dec. 4, 2003

(54) Compositions, methods and kits relating to behab and primary CNS tumors

(75) Inventors: Susan Hockfield, New Haven, CT (US); Russell T. Matthews, New Haven, CT (US); Mariano S. Viapiano, New Haven, CT (US)

Correspondence Address: MORGAN, LEWIS & BOCKIUS LLP, 1701 MARKET STREET, PHILADELPHIA, PA, 19103-2921, US

(73) Assignee: Yale University

(21) Appl. No.: 10/453,420

(22) Filed: Jun. 2, 2003

Related U.S. Application Data
(63) Continuation-in-part of application No. 10/195,970, filed on Jul. 16, 2002.
(60) Provisional application No. 60/306,046, filed on Jul. 17, 2001.

(51) Int. Cl. A61K 38/17; G01N 33/574; C07H 21/04; C12P 21/02; C12N 5/06; C07K 14/47

(52) U.S. Cl. 514/8; 435/7.23; 435/320.1; 435/325; 435/69.1; 530/395; 530/388.8; 536/23.5

(57) ABSTRACT

The present invention relates to primary CNS tumors and provides useful compositions and methods for reducing tumor volume and increasing the length of survival in mammals with primary CNS tumors, thereby providing a treatment for primary CNS tumors. The invention also relates to methods of identifying compounds for reducing tumor volume and increasing animal survival, which therefore relate to treating primary CNS tumors. The invention also relates to methods of detecting and diagnosing a tumor.

### CROSS-REFERENCE TO RELATED APPLICATIONS

This application is a continuation-in-part of U.S. application Ser. No. 10/195,970, filed Jul. 16, 2002, which is entitled to priority pursuant to 35 U.S.C. §119(e) to U.S. Provisional Patent Application No. 60/306,046, filed on Jul. 17, 2001, all of which are incorporated herein by reference in their entirety.

### STATEMENT REGARDING FEDERALLY SPONSORED RESEARCH OR DEVELOPMENT

This invention was supported in part by funds obtained from the U.S. Government (National Institutes of Health Grant Numbers EY06511 and NS35228), and the U.S. Government may therefore have certain rights in the invention.

### BACKGROUND OF THE INVENTION

Gliomas, primary brain tumors, are notoriously difficult to control and manage. Unlike secondary tumors that have metastasized to the brain or non-glial brain tumors, gliomas demonstrate a unique invasive ability, characterized by a lack of well-defined borders between cancerous tissue and healthy brain. Conventional therapies including surgery, chemotherapy, and radiation therapy, are often only partially effective or ineffective treatments due to the invasive nature of gliomas. Therefore, the prognosis of patients afflicted with gliomas is uniformly grim.

Not only is the prognosis often negative, but the diagnosis of gliomas is a difficult and costly process as well. Detection of brain tumors requires costly imaging equipment that is not readily available at all medical facilities, and confirmation of imaging results usually requires invasive and dangerous surgical sampling of the tumor.

The nature of the extracellular matrix (ECM) in the brain may play a role in the invasive capabilities of cancer cells. During normal development, the composition of the extracellular matrix of the brain changes dramatically. Cell proliferation, migration, neuronal and glial outgrowth and angiogenesis in the developing brain take place in a soluble matrix permissive to cell movement. In the mature, developed brain however, cell motility is markedly decreased to stabilize mature cell to cell interactions (Hockfield, 1990, Semin. Dev. Biol. 1:55-63).

The mechanisms employed by malignant tumors to invade the surrounding tissue in the developed brain differ remarkably. For example, some malignant cells are capable of producing their own ECM molecules, such as hyaluronic acid (HA), thereby changing the balance and structure of the neighboring environment (Delpech et al., 1997, International Journal of Cancer 72:942-948; Kosaki et al., 1999, Cancer Research, 59: 1141-1145; Turley, 1992, Cancer and Metastasis Reviews 11:21-30; Tzanakakis et al., 1997 Biochimie. 79: 323-332; Zetter, 1993, Seminars in Cancer Biology 4: 219-229). Additionally, tumor cells can alter their interactions with ECM molecules by changing the composition of their cellular receptors, for example, the upregulation of the HA binding molecules RHAMM and CD44 (Goldbrunner et al., 1999, Acta Neurochirurgica 141: 295-305; Hall and Turley, 1995, Journal of Neuro-Oncology 26: 221-229; Hall et al., 1995, Cell 82: 19-28; Merzak et al., 1994, Cancer Research 54: 3988-3992). Further, malignant cells can degrade the existing normal matrix by producing proteolytic enzymes to digest the surrounding ECM (Furcht et al., 1994, Laboratory Investigation 70: 781-783; Mignatti and Rilkin, 1993, Physiological Reviews 73: 161-195; Stetler-Stevenson et al., 1993, FASEB Journal 7: 1434-1441). While the mechanisms that facilitate the invasion of tumor cells into adjacent tissue are diverse, it is perhaps the ability of malignant tumors to modify the composition of the surrounding matrix, including the production and digestion of matrix molecules, that best characterizes the invasive phenotype.

The role of proteases in the invasive process has been demonstrated in tumors of almost every tissue type (Furcht et al., 1994, Laboratory Investigation 70: 781-783; Mignatti and Rifkin, 1993, Physiological Reviews 73: 161-195; Stetler-Stevenson et al., 1993, FASEB Journal 7: 1434-1441), and have been strongly implicated in the invasive properties of high grade gliomas (DeClerck et al., 1991, Cancer Research 51: 2151-2157; Mohanam et al., 1994, Journal of Neuro-Oncology 22: 153-160; Nakagawa et al., 1994, Journal of Neurosurgery 81: 69-77; Rao et al., 1993, Cancer Research 53: 2208-2211; Rao et al., 1994, Journal of Neuro-Oncology 18: 129-138; Vaithilingham et al., 1992, Journal of Neurosurgery 77: 595-600; Yamamoto et al., 1994, Journal of Neuro-Oncology 22: 139-151). A wealth of literature has demonstrated that matrix metalloproteinases (MMPs), especially MMP-2 and MMP-9, are highly upregulated in gliomas, and that inhibition of these proteases can decrease glioma invasiveness (Deryugina et al., 1997, Journal of Cell Science 110: 2473-2482; Forsyth et al., 1999, British Journal of Cancer 79: 1828-1835; Hamasuna et al., 1999, International Journal of Cancer 82: 274-281; Rao et al., 1996, Clinical and Experimental Metastasis 14: 12-18; Sawaya et al., 1996, Clinical and Experimental Metastasis 14: 35-42; Uhm et al., 1996, Clinical and Experimental Metastasis 14: 421-433; Nakagawa et al., 1994, Journal of Neurosurgery 81: 69-77; Rao et al., 1993, Cancer Research 53: 2208-2211; Rao et al., 1994, Journal of Neuro-Oncology 18: 129-138). However, clinical trials employing MMP inhibitors for the treatment of primary tumors have been disappointing due to serious and deleterious side effects (Heath and Grochow, 2000, Drugs 59: 1043-1055).

One of the brain ECM molecules that plays an important role in glioma invasiveness and tumor progression is brain-enriched hyaluronan binding (BEHAB) protein (Hockfield et al., 1997, U.S. Pat. No. 5,635,370). BEHAB is a member of the proteoglycan tandem-repeat family of proteins, and exists as a both a secreted and GPI-anchored protein with a hyaluronan binding domain (Jaworski et al., 1994, J. Cell Biol. 125: 495-509; Yamada et al., 1994, J. Biol. Chem 269:10119-10126; Seidenbecher et al., 1995, J. Biol. Chem. 270: 27206-27212). Recent studies have demonstrated that BEHAB is a brain specific protein that is down-regulated as development progresses in the human brain (Gary et al., 2000, Gene 256: 139-147). Importantly, BEHAB is upregulated about 700% in almost all adult human glioma samples assayed to date (Jaworski et al., 1996, Cancer Research, 56: 2293-2298, Gary et al., 2000, Gene 256: 139-147).

BEHAB cleavage plays a prominent role in the progression of glioma. Recent work by Matthews et al. (2000, J. Biol. Chem. 275: 22695-22703) demonstrated that BEHAB is cleaved between Glu³⁹⁵-Ser³⁹⁶ into 50 kDa and 90 kDa fragments by a metalloproteinase, but not by an MMP. Instead, BEHAB is cleaved by a member of the a disintegrin and metalloproteinase with thrombospondin motifs (ADAMTS) family of metalloproteinases, specifically, ADAMTS4.

Unfortunately, due to deleterious side effects, traditional metalloproteinase inhibitors have proved to be an ineffective method for treating gliomas. Given the. inherent risks of both diagnosing and treating gliomas with conventional techniques, and the failure of newer treatment regimens such as metalloproteinase inhibitors, there exists a long felt need for a method to both diagnose and treat primary central nervous system (CNS) tumors. The present invention meets this need.

### BRIEF SUMMARY OF THE INVENTION

The invention includes an isolated rat glycosylation-variant BEHAB isoform, wherein the glycosylation-variant BEHAB isoform has a molecular weight of about 130 kDa.

In one aspect the glycosylation-variant BEHAB isoform is encoded by the isolated nucleic acid of SEQ ID NO: 5.

The invention includes an isolated rat underglycosylated BEHAB isoform, wherein the underglycosylated BEHAB isoform has a molecular weight of about 130 kDa.

In one aspect the underglycosylated BEHAB isoform is encoded by the isolated nucleic acid of SEQ ID NO: 5.

The invention includes an isolated rat unglycosylated BEHAB isoform, wherein the unglycosylated BEHAB isoform has a molecular weight of about 130 kDa.

In one aspect the unglycosylated BEHAB isoform is encoded by the isolated nucleic acid of SEQ ID NO: 5.

The invention includes an isolated human glycosylation-variant BEHAB isoform, wherein the glycosylation-variant BEHAB isoform has a molecular weight of about 150 kDa.

In one aspect, the human glycosylation-variant BEHAB isoform is encoded by the isolated nucleic acid of SEQ ID NO: 7.

The invention includes an isolated human underglycosylated BEHAB isoform, wherein the underglycosylated BEHAB isoform has a molecular weight of about 150 kDa.

In one aspect, the human underglycosylated BEHAB isoform is encoded by the isolated nucleic acid of SEQ ID NO: 7.

The invention includes an isolated human unglycosylated BEHAB isoform, wherein the unglycosylated BEHAB isoform has a molecular weight of about 150 kDa.

In one aspect, the human unglycosylated BEHAB isoform is encoded by the isolated nucleic acid of SEQ ID NO: 7.

The invention includes a method of making a glycosylation-variant BEHAB isoform. The method comprises transfecting a cell with an isolated nucleic acid encoding a BEHAB protein and isolating a glycosylation-variant BEHAB therefrom.

In one aspect, the cell is an Oli-neu cell.

In another aspect, the isolated nucleic acid encoding BEHAB protein comprises the isolated nucleic acid of SEQ ID NO:5.

The invention includes a method of detecting a glycosylation-variant BEHAB isoform in a mammal. The method comprises contacting a biological sample of the mammal with an antibody that specifically binds with a glycosylation-variant BEHAB isoform or fragment thereof and detecting the binding of the antibody to the biological sample, where binding of the antibody with the sample detects a glycosylation-variant BEHAB isoform in a mammal.

In one aspect, the mammal is a human.

In another aspect, the antibody is selected from the group consisting of B5, B6, or B_{CRP}.

In yet another aspect, the antibody comprises a tag polypeptide covalently linked thereto.

The invention includes a method of diagnosing a primary CNS tumor in a mammal. The method comprises obtaining a biological sample from a mammal suspected of having a primary CNS tumor, assessing the level of a glycosylation-variant BEHAB isoform in the biological sample, and comparing the level of a glycosylation-variant BEHAB isoform in the biological sample with the level of a glycosylation-variant BEHAB isoform in a biological sample obtained from a mammal not suspected of having a primary CNS tumor. A higher level of a glycosylation-variant BEHAB isoform in the biological sample from the mammal suspected of having a primary CNS tumor compared with the level of a glycosylation-variant BEHAB isoform in the biological sample from the mammal not suspected of having a primary CNS tumor is an indication that the mammal suspected of having a primary CNS tumor has a primary CNS tumor, thereby diagnosing a primary CNS tumor in a mammal.

In one aspect, the mammal is a human.

In yet another aspect, the biological sample is selected from the group consisting of blood, neural tissue, cerebrospinal fluid, urine, saliva and brain tissue.

The invention includes a method of treating a primary CNS tumor in a mammal. The method comprises administering to a mammal an effective amount of a glycosylation-variant BEHAB isoform inhibitor, thereby treating a primary CNS tumor in a mammal.

In one aspect, the mammal is a human.

In yet another aspect, the glycosylation-variant BEHAB isoform inhibitor is selected from the group consisting of an antibody, a protein and a peptidomimetic.

In still another aspect, the antibody specifically binds to a glycosylation-variant BEHAB isoform, or fragment thereof.

The invention includes a method of assessing the effectiveness of a treatment for a primary CNS tumor in a mammal. The method comprises assessing the level of a glycosylation-variant BEHAB isoform in the mammal before, during, or after administration of a treatment for a primary CNS tumor to the mammal, where a lower level of the glycosylation-variant BEHAB isoform in the mammal during or after administration of the treatment for a primary CNS tumor with the level of the glycosylation-variant BEHAB isoform in the mammal before administration of the treatment for a primary CNS tumor is an indication of the effectiveness of the treatment for a primary CNS tumor in the mammal, thereby assessing the effectiveness of the treatment for a primary CNS tumor in said mammal.

In one aspect, the mammal is a human.

In yet another aspect, the treatment for a primary CNS tumor is selected from the group consisting of chemotherapy, radiation therapy, and surgery.

The invention includes a method of identifying a compound that affects expression of a glycosylation-variant BEHAB isoform in a cell. The method comprises contacting a cell with a test compound and comparing the level of glycosylation-variant BEHAB isoform expression in the cell with the level of glycosylation-variant BEHAB isoform expression in an otherwise identical cell not contacted with the test compound, where a higher or lower level of glycosylation-variant BEHAB isoform expression in the cell contacted with the test compound compared with the level of glycosylation-variant BEHAB isoform expression in the otherwise identical cell not contacted with the test compound is an indication that the test compound affects expression of the glycosylation-variant BEHAB isoform in a cell, thereby identifying a compound that affects expression of the glycosylation-variant BEHAB isoform in a cell.

In one aspect, the invention includes a compound identified by the method above.

The invention includes a method of identifying a compound that reduces expression of a glycosylation-variant BEHAB isoform in a cell. The method comprises contacting a cell with a test compound and comparing the level of glycosylation-variant BEHAB isoform expression in the cell with the level of glycosylation-variant BEHAB isoform expression in an otherwise identical cell not contacted with the test compound, where a higher or lower level of glycosylation-variant BEHAB isoform expression in the cell contacted with the test compound compared with the level of glycosylation-variant BEHAB isoform expression in the otherwise identical cell not contacted with the test compound is an indication that the test compound reduces expression of the glycosylation-variant BEHAB isoform in a cell, thereby identifying a compound that reduces expression of the glycosylation-variant BEHAB isoform in a cell.

In one aspect, the invention includes a compound identified by the method above.

The invention includes a method of treating a primary CNS tumor in a mammal. The method comprises isolating a cell from a mammal, contacting the cell with a glycosylation-variant BEHAB isoform, or a fragment thereof, and administering the cell so contacted to the mammal.

In one aspect, the cell is an antigen presenting cell.

In another aspect, the cell is a dendritic cell.

The invention includes a kit for detecting a glycosylation-variant BEHAB isoform. The kit comprises an-antibody to a glycosylation-variant BEHAB isoform. The kit further comprises an instructional material for the use thereof.

The invention includes a kit for diagnosing a primary CNS tumor in a mammal. The kit comprises an antibody to a glycosylation-variant BEHAB isoform. The kit further comprises an applicator, and an instructional material for the use thereof.

The invention includes a kit for treating a primary CNS tumor. The kit comprises a composition comprising an antibody that specifically binds with a glycosylation-variant BEHAB isoform, or a fragment thereof, and a pharmaceutically acceptable carrier. The kit further comprises an applicator, and an instructional material for use thereof.

The invention includes a kit for treating a primary CNS tumor with immune therapy. The kit comprises a glycosylation-variant BEHAB isoform, or a fragment thereof, the kit further comprising an applicator, and an instructional material for use thereof.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A through 1C, depicts images of Western blot analysis of media from cells stably (FIG. 1C) or transiently (FIG. 1A and 1B) transfected with full-length and/or mutant BEHAB. FIG. 1A depicts Western blots of media from cells transiently transfected with full-length (FL) and mutant (NVY) BEHAB. Both cell lines exhibit strong staining of the 145 kDa full-length protein. In contrast, no cleavage product was detected in the NVY mutant using the B50 antibody, while a 50 kDa cleavage product was detected in the media of cells transfected with the FL. FIG. 1B depicts Western blots of media from cells transiently co-transfected with 2 μg of the FL construct and 0, 1, 2, or 4 μg of the NVY mutant construct. Transfection of the NVY mutant increased the amount of full-length BEHAB, but had no effect on cleavage of normal BEHAB as seen using the B50 antibody. FIG. 1C depicts Western blots of media from cells stably transfected with full-length (FL) and mutant (NVY) BEHAB. Expression of BEHAB was examined in the CNS-1-FL and CNS-1-NVY cells using the B6 and B50 antibodies. As with the transient transfections, cells stably transfected with CNS-1-FL produced and cleaved BEHAB, whereas cells stably transfected with CNS-1-NVY produced the full-length protein but did not cleave it.

FIG. 2, comprising FIGS. 2A and 2B, depicts the effects of stable transfection on cell proliferation and cell death. CNS-1-FL, CNS-1-GFP, and CNS-1-NVY stable cell lines were compared to parental CNS-1 cells for the effect of CNS-1-FL, CNS-1-GFP, and CNS-1-NVY transfection on cell proliferation (FIG. 2A) and cell death (FIG. 2B). FIG. 2A is a graph depicting cell proliferation over seven days using the MTT assay. Data show changes in absorbance. Transfection using CNS-1-FL, CNS-1-GFP, and CNS-1-NVY had no effect on cell proliferation. FIG. 2B is a graph depicting cell death over seven days as evaluated using the LDH assay. Transfections using CNS-1-FL, CNS-1-GFP, and CNS-1-NVY had no effect on cell death.

FIG. 3, comprising FIGS. 3A through 3D, depicts the effect of transfection using CNS-1-FL, CNS-1-GFP, and CNS-1-NVY on tumor volume. Stably transfected cells were implanted intracranially in rats for eight days, and the relative sizes of tumors that resulted were evaluated histologically. FIG. 3A depicts an image of a representative tumor derived from CNS-1-GFP cells. FIG. 3B depicts an image of a representative tumor derived from CNS-1-FL cells. FIG. 3C depicts an image of a representative tumor derived from CNS-1-NVY cells. The CNS-1-FL tumor is larger and more invasive than the CNS-1-GFP and CNS-1-NVY tumors. FIG. 3D is a graph depicting volumes of tumors derived from CNS-1-FL, CNS-1-GFP, and CNS-1-NVY cells. These data were quantified by image analysis and volume reconstruction, and individual data points are represented by an asterisk.

FIG. 4 is a graph depicting the effect of tumors derived from cells stably transfected with CNS-1-FL, CNS-1-GFP, and CNS-1-NVY on animal survival. Animals with CNS-1-FL tumors survived a significantly shorter time than animals with CNS-1-GFP tumors or CNS-1-NVY tumors. Survival in animals with CNS-1-NVY tumors was not significantly different from survival in animals with CNS-1-GFP control tumors.

FIG. 5, comprising FIGS. 5A and 5B, is a series of images depicting expression of a BEHAB isoform over the embryonic (e), postnatal (P) and adult (Ad) developmental stages in rats. Total homogenates from rat brains were separated into soluble(s) and membrane particulate (p) fractions. The resulting fractions were treated with chondroitinase ABC and processed for western blotting (FIG. 5A). Full-length BEHAB (B/b_{FL}) and 90 kDa BEHAB cleavage product (B/b₉₀) were detected with B6 antibody, 50 kDa cleavage product (B/b₅₀) was detected with B50 antibody (FIG. 5B). Arrows indicate the positions of the full-length BEHAB isoform (B/b_{FL}) as well as the membrane-associated glycosylation-variant BEHAB isoform (B/b_{Δg}). FIG. 6, comprising FIGS. 6A through 6D, is series of images depicting the difference between GPI-anchored BEHAB and glycosylation-variant BEHAB. FIG. 6A depicts adult rat brain total membranes (M) supernatant (s) and pellet (p) resuspended in TrisHCl buffer, (Tris), Tris buffer with Triton X-100 (Tx100), or Na₂CO₃, (Na₂CO₃). FIG. 6B depicts adult rat brain membranes resuspended in CH buffer with chondroitinase ABC in the absence (Ctrl) or presence of PI-PLC (PI_PLC). A new 120-kDa band, representing the GPI-anchored isoform, was detected following PI-PLC treatment. FIG. 6C depicts rat brain membranes treated enzymatically as above, and subsequently separated by centrifugation. FIG. 6D depicts rat brain membranes resuspended in CH buffer (S) and extracted with Triton X-114, yielding an insoluble pellet (In) an aqueous phase (H₂O) and a detergent phase (Tx). Arrows indicate full-length BEHAB (B/b_{FL}), glycosylation-variant BEHAB (B/b_{Δg}) and GPI-linked BEHAB isoforms.

FIG. 7, comprising FIGS. 7A through 7D, is a series of images demonstrating that glycosylation-variant BEHAB is not GPI-linked BEHAB. FIG. 7A is a schematic diagram depicting the structure of BEHAB and the location of the immunogenic epitopes recognized by antibodies B6, B5 and B_{CRP}. HABD, HA-binding domain; GAG, GAG-attachment region; EGF, epidermal growth factor repeat; CRP, complement regulatory protein-like domain. FIG. 7B is an image of a blot depicting solubilized rat brain membranes (M) immunoprecipitated in the absence (mock) or presence (B6) of B6 antibody. Immunoprecipitated samples were immuno-detected using B6, B5 and B_{CRP} antibodies. FIG. 7C is an image of a blot depicting culture medium (m) and cell membranes (c) from the CNS-1 rat glioma cell line stably transfected with a full-length rat BEHAB cDNA and detected with B6, B5 and B_{CRP} antibodies. Again, all antibodies detected full-length BEHAB and glycosylation-variant BEHAB, indicating that the size differences between these two bands is not generated by cleavage. The asterisk indicates a band corresponding to a C-terminally-clipped degradation product of BEHAB, not detected by the B_{CRP} antibody. FIG. 7D is an image depicting culture medium (m) and cell membranes (c) from mouse Oli-neu cells expressing untagged or V5/6xHis-tagged BEHAB cDNA and immunoblotted respectively with B6 or V5 antibodies. In both cases, only the rat glycosylation-variant BEHAB isoform was detected in these cells.

FIG. 8 is an image depicting that full-length BEHAB and glycosylation-variant-BEHAB arise from differential glycosylation of a single core protein. Arrows indicate full-length BEHAB (B/b_{FL}) and glycosylation variant BEHAB (B/b_{Δg}) isoforms.

FIG. 9, comprising FIGS. 9A and 9B, is a series of images depicting the calcium-independent association of glycosylation-variant BEHAB with cell membranes. FIG. 9A depicts total membranes (M) from adult rat brain and FIG. 9B depicts total membranes from Oli-neu cells transfected with V5/6xHis-BEHAB cDNA and resuspended in TrisHCl buffer, with (EDTA) or without EDTA (Tris). The resulting supernatant (s) and pellet fractions (p) were treated with chondroitinase ABC and immunoblotted with B6 (rat brain) or V5 (Oli-neu cells) antibodies. Arrows indicate the full-length BEHAB (B/b_{FL}) and glycosylation-variant BEHAB (B/b_{Δg}) isoforms.

FIG. 10 is an image depicting the enrichment of glycosylation-variant BEHAB in light membrane subcellular fractions. Total rat brain homogenate (H) was subjected to subcellular fractionation and the resulting fractions were treated with chondroitinase ABC prior to western blotting with B6 antibody. Fractions in FIG. 10 are: nuclear pellet (Nuc), heavy mitochondrial pellet (HM), light microsomal pellet (Mc) and soluble supernatant (Sol). The heavy mitochondrial pellet was subfractionated in a discontinuous sucrose gradient in the following subfractions: floating myelin fraction (My), light membrane fraction (LM), synaptosomal fraction (Syn) and mitochondrial pellet (Mit). Arrows indicate the full-length BEHAB (B/b_{FL}) and glycosylation-variant BEHAB (B/b_{Δg}) isoforms.

FIG. 11 is a series of images depicting the location of glycosylation-variant BEHAB on the cell surface. Oli-neu cells transfected with V5/His-tagged rat BEHAB cDNA were live stained with an anti-V5 antibody (V5) to detect tagged glycosylation-variant BEHAB. Cells were counterstained with propidium iodide (PI). FIG. 11A depicts BEHAB-transfected cells stained with anti-V5, demonstrating the extracellular location of glycosylation-variant BEHAB. FIG. 11B depicts control (vector)-transfected cells stained with anti-V5; and FIG. C depicts BEHAB-transfected cells stained with non-immune serum. Bar=20 μm.

FIG. 12 is an image depicting the upregulation of glycosylation-variant BEHAB in a rat model of glioma. A sample from rat brain glioma tumor produced by intracranial injection of transfected CNS-1cells (Gli) was fractionated into soluble (s) and particulate (p) fractions, treated with chondroitinase ABC and processed for western blotting. A paired control sample (Ctrl) corresponding to the contralateral side of the brain, as well as a separate control sample from non-treated adult animals (Ad) were processed identically. BEHAB was detected using the B6 antibody. Arrows indicate the full-length BEHAB (B/b_{FL}) and glycosylation-variant BEHAB (B/b_{Δg}).

FIG. 13 is an image depicting immunoblots demonstrating expression of a BEHAB isoform across human development in post-mortem brain tissue from 55 days (d) to 72 years (yr) of age and in surgical glioma (Gli) samples. (S) indicates the soluble fraction from human brain homogenate and (P) indicates the membrane particulate fractions of human brain homogenate. The resulting fractions were treated with chondroitinase ABC and processed for western blotting. Human full-length BEHAB (B/b_{FL}) was detected with the B6 antibody. Arrows indicate the positions of full-length BEHAB isoform (B/b_{FL}) and the membrane-associated glycosylation-variant BEHAB isoform (B/b_{Δg}).

FIG. 14 is an image of immunoblots depicting that human glioma-specific glycosylation-variant BEHAB (B/b_{Δg}) is not a product of proteolytic cleavage of the full-length BEHAB isoform. Solubilized membranes (M) from normal human brains and gliomas were immunoprecipitated in the absence (mock) or presence (B6) of B6 antibody. Immunoprecipitated samples were immuno-detected using B6, B5 and B_{CRP} antibodies. Arrows indicate the positions of a full-length BEHAB isoform (B/b_{FL}) and a membrane-associated glycosylation-variant BEHAB isoform (B/b_{Δg}).

FIG. 15 is an image depicting that human full-length BEHAB and glioma-specific glycosylation-variant BEHAB arise from differential glycosylation of a single core protein. Samples were treated with chondroitinase (CH'ase) PNGase F (N-glyc F), O-glycosidase (O-glycos.) and/or sialidase. Arrows indicate full-length BEHAB (B/b_{FL}) and glycosylation-variant BEHAB (B/b_{Δg}).

FIG. 16 is an image depicting the calcium-independent association of glycosylation-variant BEHAB with cell membranes. Total membranes (M) from adult human brain and glioma were resuspended in TrisHCl buffer, with (EDTA) or without EDTA (Tris). The resulting supernatant (s) and pellet fractions (p) were treated with chondroitinase ABC and immunoblotted with the B6 antibody. Arrows indicate the full-length BEHAB (B/b_{FL}) and glycosylation-variant BEHAB (B/b_{Δg}) isoforms.

### DETAILED DESCRIPTION OF THE INVENTION

Manipulation of the extracellular matrix (ECM) of the brain plays an important role in the progression and invasive phenotype of primary CNS tumors. One component of the ECM, brain-enriched hyaluronan binding (BEHAB) protein is vital in glioma cell motility, and thus invasiveness. The data disclosed herein demonstrate for the first time, that affecting BEHAB function and/or cleavage mediates a decrease in tumor size and an increase in survival time in animals with primary CNS tumors. Therefore, the present invention includes compositions and methods for the treatment of primary CNS tumors, including, but not limited to gliomas, oligodendroglioma, astrocytoma, gliosarcoma, glioblastoma multiforme, lymphoma, and reactive gliosis following brain injury.

The present invention also includes compositions and methods for diagnosing primary CNS tumors in a mammal. That is, the data disclosed herein demonstrate that a primary CNS tumor, including, but not limited to, gliomas, oligodendroglioma, astrocytoma, and gliosarcoma, may be diagnosed in a mammal using the methods disclosed herein.

The present invention further encompasses a novel glycosylation-variant BEHAB isoform. The data disclosed herein demonstrate for the first time that this novel glycosylation-variant isoform binds to the cell-membrane in a previously undisclosed calcium-independent manner, and is dramatically upregulated in invasive gliomas. Thus, the present invention includes compositions and methods for the generation of a glycosylation-variant BEHAB isoform, as well as compositions, methods and kits for the detection, treatment, and diagnosis of primary CNS tumors relating to a glycosylation-variant BEHAB isoform.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element. “Amplification” refers to any means by which a polynucleotide sequence is copied and thus expanded into a larger number of polynucleotide molecules, e.g., by reverse transcription, polymerase chain reaction, and ligase chain reaction.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

By the term “applicator” as the term is used herein, is meant any device including, but not limited to, a hypodermic syringe, a pipette, and the like, for administering the mutant BEHAB nucleic acid, protein, and/or anti-BEHAB antibodies and the antisense BEHAB nucleic acid of the invention to a mammal.

“BEHAB” “full-length BEHAB”, or “endogenous BEHAB” as the terms are used synonymously herein, refers to the Brain-Enriched Hyaluronan Binding molecule, otherwise known as brevican. Full-length BEHAB has a molecular weight of greater than about 150 kDa in rats and mice, and greater than about 160 kDa in humans and is exemplified by the nucleotide and amino acid sequences set forth in SEQ ID NO:5 SEQ ID NO:6 for rat full-length BEHAB, and SEQ ID NO:7 and SEQ ID NO:8 for human full-length BEHAB, respectively.

“Biological sample,” as that term is used herein, means a sample obtained from or in a mammal that can be used to assess the level of expression of a BEHAB, the level of BEHAB protein present, or both. Such a sample includes, but is not limited to, a blood sample, a neural tissue sample, a brain sample, and a cerebrospinal fluid sample.

“Cleavage” is used herein to refer to the disassociation of a peptide bond between two amino acids in a polypeptide, thereby separating the polypeptide comprising the two amino acids into at least two fragments.

A “cleavage inhibitor” is used herein to refer to a molecule, compound or composition that prevents the cleavage of a polypeptide either by titrating the protease responsible for cleavage, blocking the cleavage site, or otherwise making the cleavage site unrecognizable to a protease.

“Cleavage inhibiting amount” is used herein to refer to an effective amount of a cleavage inhibitor.

“Cleavage products” is used herein to refer to the fragments of an initial polypeptide resulting from the cleavage of the initial polypeptide into two or more fragments. As an example, the cleavage products of the 145 kDa BEHAB protein include 90 kDa and 50 kDa fragments.

By “complementary to a portion or all of the nucleic acid encoding BEHAB” is meant a sequence of nucleic acid which does not encode a BEHAB protein. Rather, the sequence which is being expressed in the cells is identical to the non-coding strand of the nucleic acid encoding a BEHAB protein and thus, does not encode BEHAB protein.

The terms “complementary” and “antisense” as used herein, are not entirely synonymous. “Antisense” refers particularly to the nucleic acid sequence of the non-coding strand of a double stranded DNA molecule encoding a protein, or to a sequence which is substantially homologous to the non-coding strand.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anticodon region of a transfer RNA molecule during translation of the mRNA molecule or which encode a stop codon. The coding region may thus include nucleotide residues corresponding to amino acid residues which are not present in the mature protein encoded by the mRNA molecule (e.g. amino acid residues in a protein export signal sequence).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A first region of an oligonucleotide “flanks” a second region of the oligonucleotide if the two regions are adjacent one another or if the two regions are separated by no more than about 1000 nucleotide residues, and preferably no more than about 100 nucleotide residues.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 500 nucleotides, even more preferably, at least about 500 nucleotides to about 1000 nucleotides, yet even more preferably, at least about 1000 to about 1500, even more preferably, at least about 1500 nucleotides to about 2000 nucleotides, yet even more preferably, at least about 2000 to about 2500, even more preferably, at least about 2500 nucleotides to about 2600 nucleotides, yet even more preferably, at least about 2600 to about 2650, and most preferably, the nucleic acid fragment will be greater than about 2652 nucleotides in length.

As applied to a protein, a “fragment” of BEHAB is about 20 amino acids in length. More preferably, the fragment of a BEHAB is about 100 amino acids, even more preferably, at least about 200, yet more preferably, at least about 300, even more preferably, at least about 400, yet more preferably, at least about 500, even more preferably, about 600, and more preferably, even more preferably, at least about 700, yet more preferably, at least about 800, even more preferably, about 850, and more preferably, at least about 884 amino acids in length.

As used herein, a “glycosylation-variant BEHAB isoform” and “glycosylation-variant BEHAB” means a BEHAB protein having an altered glycosylation pattern as compared to the glycosylation pattern of full-length BEHAB and a molecular weight less than about 150 kDa in rats and less than about 160 kDa in humans. The term glycosylation-variant BEHAB isoform or glycosylation-variant BEHAB includes underglycosylated BEHAB, differently-glycosylated BEHAB and unglycosylated BEHAB.

As used herein, a “differently-glycosylated BEHAB” and a “differently-glycosylated BEHAB isoform” refers to a BEHAB protein having an altered glycosylation pattern wherein the carbohydrate and sugar content is similar to that of full-length BEHAB, but the composition of the sugars associated with the amino acid backbone is altered.

“Underglycosylated BEHAB isoform” and “underglycosylated BEHAB” are used herein to refer to a BEHAB protein having the primary amino acid sequence of a full-length BEHAB protein, or a fragment thereof, but having less than the glycosylation content of the full-length BEHAB protein, but still having at least one sugar or carbohydrate associated with the protein.

“Unglycosylated BEHAB isoform” and “unglycosylated BEHAB” are used herein to refer to a BEHAB protein having the primary amino acid sequence of a full-length BEHAB protein, or fragment thereof, but having no sugars or carbohydrates associated with the protein.

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g, as a cDNA or a genomic or cDNA fragment produced by PCR or restriction enzyme digestion) independent of other sequences. It also includes a recombinant DNA which is part of a hybrid gene encoding additional polypeptide sequence.

“Mutant BEHAB” is used herein to refer to a Brain Enriched Hyaluronan Binding molecule in which the amino acid sequence has been modified to inhibit cleavage by proteases.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is naturally-occurring.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

A “portion” of a polynucleotide means at least at least about twenty sequential nucleotide residues of the polynucleotide. It is understood that a portion of a polynucleotide may include every nucleotide residue of the polynucleotide.

“Primary CNS tumor” is used herein to refer to a neoplasia with origins in the brain, in that the cancerous cells did not originate in another part of the body and metastasize to the brain. Examples of primary CNS tumors include, but are not limited to, gliomas, well-differentiated astrocytomas, anaplastic astrocytomas, glioblastoma multiforme, ependymomas, oligodendrogliomas, ganglioneuromas, mixed gliomas, brain stem gliomas, optic nerve gliomas, meningiomas, pineal tumors, pituitary tumors, pituitary adenomas, reactive gliosis, primitive neuroectodermal tumors, schwannomas, lymphomas, vascular tumors, and lymphomas.

“Treating a primary CNS tumor” is used herein to refer to a situation where the severity of a symptom of a primary CNS tumor, including the volume of the tumor or the frequency with which any symptom or sign of the tumor is experienced by a patient, or both, is reduced, or where time to tumor progression or survival time is increased.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Probe” refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

By the term “specifically binds,” as used herein, is meant an antibody which recognizes and binds an epitope of a BEHAB protein, but does not substantially recognize or bind other molecules in a sample.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

A “transgene”, as used herein, means an exogenous nucleic acid sequence comprising a nucleic acid which encodes a promoter/regulatory sequence operably linked to nucleic acid which encodes an amino acid sequence, which exogenous nucleic acid is encoded by an animal or cell.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

Description

I. Isolated Nucleic Acids

A. Sense Nucleic Acids

The present invention includes an isolated nucleic acid encoding a mammalian mutant BEHAB molecule, or a fragment thereof, wherein the nucleic acid shares at least about 99.7% identity with a nucleic acid having the sequence of SEQ ID NO:4. The mammal is preferably a human. Preferably, the nucleic acid is about 99.8% homologous, more preferably, and most preferably, about 99.9% homologous to SEQ ID NO:4, disclosed herein. Even more preferably, the nucleic acid is SEQ ID NO:4. The isolated nucleic acid of the invention should be construed to include an RNA or a DNA sequence encoding a mutant BEHAB protein of the invention, and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

The present invention should not be construed as being limited solely to the nucleic and amino acid sequences disclosed herein. Once armed with the present invention, it is readily apparent to one skilled in the art that other nucleic acids encoding mutant BEHAB proteins can be obtained by following the procedures described herein in the experimental details section for the generation of other mammalian mutant BEHAB nucleic acids encoding mutant BEHAB polypeptides as disclosed herein (e.g., site-directed mutagenesis, frame shift mutations, and the like), and procedures that are well-known in the art or to be developed.

Further, any other number of procedures may be used for the generation of derivative or variant forms of mutant BEHAB using recombinant DNA methodology well known in the art such as, for example, that described in Sambrook et al. (1989, Molecular Cloning: A Laboratory Manual, Cold Spring Harbor Laboratory Press, New York) and Ausubel et al. (1997, Current Protocols in Molecular Biology, Green & Wiley, New York).

Procedures for the introduction of amino acid changes in a protein or polypeptide by altering the DNA sequence encoding the polypeptide are well known in the art and are also described in Sambrook et al. (1989, supra); Ausubel et al. (1997, supra).

The invention includes a nucleic acid encoding a mammalian mutant BEHAB wherein a nucleic acid encoding a tag polypeptide is covalently linked thereto. That is, the invention encompasses a chimeric nucleic acid wherein the nucleic acid sequence encoding a tag polypeptide is covalently linked to the nucleic acid encoding a mutant BEHAB polypeptide. Such tag polypeptides are well known in the art and include, for instance, green fluorescent protein (GFP), myc, myc-pyruvate kinase (myc-PK), His₆, maltose biding protein (MBP), an influenza virus hemagglutinin tag polypeptide, a flag tag polypeptide (FLAG), and a glutathione-S-transferase (GST) tag polypeptide. However, the invention should in no way be construed to be limited to the nucleic acids encoding the above-listed tag polypeptides. Rather, any nucleic acid sequence encoding a polypeptide which may function in a manner substantially similar to these tag polypeptides should be construed to be included in the present invention.

The nucleic acid comprising a nucleic acid encoding a tag polypeptide can be used to localize mutant BEHAB within a cell, a tissue, and/or a whole organism (e.g., a mammalian embryo), detect mutant BEHAB secreted from a cell, and to study the role(s) of mutant BEHAB in a cell. Further, addition of a tag polypeptide facilitates isolation and purification of the “tagged” protein such that the proteins of the invention can be produced and purified readily.

B. Antisense nucleic acids

In certain situations, it may be desirable to inhibit expression of BEHAB and the invention therefore includes compositions useful for inhibition of BEHAB expression. Thus, the invention features an isolated nucleic acid complementary to a portion or all of a nucleic acid encoding a mammalian BEHAB molecule which nucleic acid is in an antisense orientation with respect to transcription. Preferably, the antisense nucleic acid is complementary with a nucleic acid having at least about 99.7% homology with SEQ ID NO:5, or a fragment thereof. Preferably, the nucleic acid is about 99.8% homologous, and most preferably, about 99.9% homologous to a nucleic acid complementary to a portion or all of a nucleic acid encoding a mammalian BEHAB having the sequence of SEQ ID NO:5, or a fragment thereof, which is in an antisense orientation with respect to transcription. Most preferably, the nucleic acid is complementary to a portion or all of a nucleic acid that is SEQ ID NO:5, or a fragment thereof. Such antisense nucleic acid serves to inhibit the expression, function, or both, of a BEHAB molecule.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see Cohen, 1989, In: Oligodeoxyribonucleotides, Antisense Inhibitors of Gene Expression, CRC Press, Boca Raton, Fla.; Tullis, 1991, U.S. Pat. No. 5,023,243,) incorporated by reference herein in its entirety.

II. Isolated Polypeptides

The invention also includes an isolated polypeptide comprising a mammalian mutant BEHAB molecule. Preferably, the isolated polypeptide comprising a mammalian mutant BEHAB molecule is at least about 99.6% homologous to a polypeptide having the amino acid sequence of SEQ ID NO:3, or some fragment thereof. Preferably, the isolated polypeptide is about 99.7% homologous, more preferably, about 99.8% homologous, more preferably, and most preferably, about 99.9% homologous to SEQ ID NO:3, or some fragment thereof. Most preferably, the isolated polypeptide comprising a mutant BEHAB molecule is SEQ ID NO:3.

The present invention also provides for analogs of proteins or peptides which comprise a mutant BEHAB molecule as disclosed herein. Analogs may differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine.

Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

The present invention should also be construed to encompass “derivatives,” and “variants” of the peptides of the invention (or of the DNA encoding the same) which derivatives and variants are mutant BEHAB peptides which are altered in one or more amino acids (or, when referring to the nucleotide sequence encoding the same, are altered in one or more base pairs) such that the resulting peptide (or DNA) is not identical to the sequences recited herein, but has the same biological property as the peptides disclosed herein, in that the peptide has biological/biochemical properties of the mutant BEHAB peptide of the present invention.

A biological property of a mutant BEHAB protein should be construed to but not be limited to include the ability of the peptide to be secreted from a cell, or anchored via a GPI-linkage, the ability to not be cleaved by a protease, and the like.

The skilled artisan would understand, based upon the disclosure provided herein, that mutant BEHAB biological activity encompasses, but is not limited to, the ability of a molecule or compound to be expressed in brain tissue, to be detected in brain tissue, to be secreted from a cell, to be anchored to a cell, to not be cleaved by a protease, and the like. “Mutant BEHAB activity” includes the effects of mutant BEHAB, either that circulating in the ECM or cerebrospinal fluid or that produced locally in the brain. Mutant BEHAB biological activity mediates, is associated with, or both, inter alia, tumor progression, tumor invasiveness, tumor volume and size, animal survival, and the like.

III. Vectors

In other related aspects, the invention includes an isolated nucleic acid encoding a mammalian mutant BEHAB operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

Expression of mutant BEHAB, either alone or fused to a detectable tag polypeptide, in cells which either normally express normal BEHAB, may be accomplished by generating a plasmid, viral, or other type of vector comprising the desired nucleic acid operably linked to a promoter/regulatory sequence which serves to drive expression of the protein, with or without tag, in cells in which the vector is introduced. Many promoter/regulatory sequences useful for driving constitutive expression of a gene are available in the art and include, but are not limited to, for example, the cytomegalovirus immediate early promoter enhancer sequence, the SV40 early promoter, both of which were used in the experiments disclosed herein, as well as the Rous sarcoma virus promoter, and the like. Moreover, inducible and tissue specific expression of the nucleic acid encoding mutant BEHAB may be accomplished by placing the nucleic acid encoding mutant BEHAB, with or without a tag, under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein operably linked thereto.

Expressing mutant BEHAB using a vector allows the isolation of large amounts of recombinantly produced protein. Further, where the expression of full-length BEHAB expression causes a disease, disorder, or condition associated with such expression, the expression of mutant BEHAB driven by a promoter/regulatory sequence can provide useful therapeutics including, but not limited to, gene therapy whereby mutant BEHAB is provided. A disease, disorder or condition associated with an increased level of expression, level of protein, or increased level of cleavage and/or activity of the protein or its cleavage products, for which administration of mutant BEHAB can be useful can include, but is not limited to, primary CNS tumors, gliomas, well-differentiated astrocytomas, anaplastic astrocytomas, glioblastoma multiforme, ependymomas, oligodendrogliomas, ganglioneuromas, mixed gliomas, brain stem gliomas, optic nerve gliomas, meningiomas, pineal tumors, pituitary tumors, pituitary adenomas, primitive neuroectodermal tumors, schwannomas, vascular tumors, lymphomas and the like.

Therefore, the invention includes not only methods of inhibiting full-length BEHAB expression, translation, cleavage and/or activity, but it also includes methods relating to decreasing BEHAB expression, protein level, cleavage and/or activity since decreasing BEHAB expression, cleavage and/or activity or increasing mutant BEHAB expression and/or activity can be useful in providing effective therapeutics.

Selection of any particular plasmid vector or other DNA vector is not a limiting factor in this invention and a wide plethora of vectors are well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

The invention thus includes a vector comprising an isolated nucleic acid encoding a mammalian mutant BEHAB. The incorporation of a desired nucleic acid into a vector and the choice of vectors is well-known in the art as described in, for example, Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

The invention also includes cells, viruses, proviruses, and the like, containing such vectors. Methods for producing cells comprising vectors and/or exogenous nucleic acids are well-known in the art. See, for example, Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

The nucleic acids encoding mutant BEHAB may be cloned into various plasmid vectors. However, the present invention should not be construed to be limited to plasmids or to any particular vector. Instead, the present invention should be construed to encompass a wide plethora of vectors which are readily available and/or well-known in the art.

IV. Recombinant Cells

The invention includes a recombinant cell comprising, inter alia, an isolated nucleic acid encoding mutant BEHAB, an antisense nucleic acid complementary thereto, a nucleic acid encoding an antibody that specifically binds BEHAB or its cleavage products, and the like. In one aspect, the recombinant cell can be transiently transfected with a plasmid encoding a portion of the nucleic acid encoding mutant BEHAB. The nucleic acid need not be integrated into the cell genome nor does it need to be expressed in the cell. Moreover, the cell may be a prokaryotic or a eukaryotic cell and the invention should not be construed to be limited to any particular cell line or cell type. Such cells include, but are not limited to, neurons, neural cells, brain cells, glioma-derived cell lines, glial cell lines, non-glial cell lines, stem cell lines, and the like.

The invention further includes a method of making a glycosylation-variant BEHAB isoform in a recombinant cell comprising, inter alia, an isolated nucleic acid encoding a BEHAB protein. That is, as demonstrated by the data disclosed herein, a glycosylation-variant BEHAB isoform can be produced in a recombinant cell by transfecting a cell with an isolated nucleic acid encoding BEHAB, or a fragment thereof, and isolating the glycosylation-variant BEHAB isoform therefrom. Cells useful for the production of a glycosylation-variant BEHAB include, for example, an Oli-neu cell. Further, methods for transfecting a cell and producing a protein therefrom are well known in the art and are described in detail elsewhere herein. Recombinant cells thus include those which express full-length BEHAB, and those that express a glycosylation-variant BEHAB.

Further, it is important to note that the purpose of recombinant cells should not be construed to be limited to the generation of intracranial tumors. Rather, the invention should be construed to include any cell type into which a nucleic acid encoding a mammalian mutant BEHAB is introduced, including, without limitation, a prokaryotic cell and a eukaryotic cell comprising an isolated nucleic acid encoding mammalian mutant BEHAB.

The invention includes a eukaryotic cell which, when the recombinant gene of the invention is introduced therein, and the protein encoded by the desired gene is expressed therefrom, where it was not previously present or expressed in the cell or where it is now expressed at a level or under circumstances different than that before the transgene was introduced, a benefit is obtained. Such a benefit may include the fact that there has been provided a system wherein the expression of the desired gene can be studied in vitro in the laboratory or in a mammal in which the cell resides, a system wherein cells comprising the introduced gene can be used as research, diagnostic and therapeutic tools, and a system wherein mammal models are generated which are useful for the development of new diagnostic and therapeutic tools for selected disease states in a mammal.

A cell expressing an isolated nucleic acid encoding mutant BEHAB can be used to provide mutant BEHAB to a cell, tissue, or whole mammal where a higher level of mutant BEHAB can be useful to treat or alleviate a disease, disorder or condition associated with full-length BEHAB expression, cleavage, and/or activity. Such diseases, disorders or conditions can include, but are not limited to, primary CNS tumors, gliomas, well-differentiated astrocytomas, anaplastic astrocytomas, glioblastoma multiforme, ependymomas, oligodendrogliomas, ganglioneuromas, mixed gliomas, brain stem gliomas, optic nerve gliomas, meningiomas, pineal tumors, pituitary tumors, pituitary adenomas, primitive neuroectodermal tumors, schwannomas, vascular tumors, lymphomas, and the like. Therefore, the invention includes a cell expressing mutant BEHAB to decrease or prevent full-length BEHAB expression, translation, cleavage and/or activity, where increasing mutant BEHAB expression, protein level, and/or activity can be useful to treat or alleviate a disease, disorder or condition.

One of ordinary skill would appreciate, based upon the disclosure provided herein, that a “knock-in” or “knock-out” vector of the invention comprises at least two sequences homologous to two portions of the nucleic acid which is to be replaced or deleted, respectively. The two sequences are homologous with sequences that flank the gene; that is, one sequence is homologous with a region at or near the 5′ portion of the coding sequence of the nucleic acid encoding full-length BEHAB and the other sequence is further downstream from the first. One skilled in the art would appreciate, based upon the disclosure provided herein, that the present invention is not limited to any specific flanking nucleic acid sequences. Instead, the targeting vector may comprise two sequences which remove some or all of, for example, full-length BEHAB (i.e., a “knock-out” vector) or which insert (i.e., a “knock-in” vector) a nucleic acid encoding mutant BEHAB, or a fragment thereof, from or into a mammalian genome, respectively. The crucial feature of the targeting vector is that it comprise sufficient portions of two sequences located towards opposite, i.e., 5′ and 3′, ends of the full-length BEHAB open reading frame (ORF) in the case of a “knock-out” vector, to allow deletion/insertion by homologous recombination to occur such that all or a portion of the nucleic acid encoding full-length BEHAB is deleted from a location on a mammalian chromosome.

The design of transgenes and knock-in and knock-out targeting vectors is well-known in the art and is described in standard treatises such as Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York), and the like. The upstream and downstream portions flanking or within the BEHAB coding region to be used in the targeting vector may be easily selected based upon known methods and following the teachings disclosed herein based on the disclosure provided herein including the nucleic and amino acid sequences of mammalian BEHAB and mutant BEHAB. Armed with these sequences, one of ordinary skill in the art would be able to construct the transgenes and knock-out vectors of the invention.

One skilled in the art would appreciate, based upon this disclosure, that cells comprising decreased levels of full-length BEHAB protein, decreased levels of BEHAB and/or BEHAB cleavage product activity, or both, include, but are not limited to, cells expressing inhibitors of BEHAB expression (e.g., antisense or ribozyme molecules, synthetic antibodies or intrabodies).

Methods and compositions useful for maintaining mammalian cells in culture are well known in the art, wherein the mammalian cells are obtained from a mammal including, but not limited to, cells obtained from a mouse, a rat, a human, and the like.

The recombinant cell of the invention can be used to study the effect of qualitative and quantitative alterations in BEHAB levels on tumor progression and invasiveness. This is because the fact that BEHAB is secreted and possesses a hyaluronan binding domain indicates that BEHAB is involved in the function, composition, or activity of the ECM. Further, the recombinant cell can be used to produce mutant BEHAB for use for therapeutic and/or diagnostic purposes. That is, a recombinant cell expressing mutant BEHAB can be used to produce large amounts of purified and isolated mutant BEHAB that can be administered to treat or alleviate a disease, disorder or condition associated with or caused by BEHAB expression, activity, and/or cleavage.

Alternatively, recombinant cells expressing mutant BEHAB can be administered in ex vivo and in vivo therapies where administering the recombinant cells thereby administers the protein to a cell, a tissue, and/or a mammal. Additionally, the recombinant cells are useful for the discovery of BEHAB receptor and BEHAB signaling pathways.

The recombinant cell of the invention, wherein the cell has been engineered such that it does not express BEHAB, or expresses mutant BEHAB lacking cleavability, can also be used in ex vivo and in vivo cell therapies where either a mammal's own cells (e.g., neural cells, brain cells, and the like) or those of a syngeneic matched donor are recombinantly engineered as described elsewhere herein (e.g., by insertion of an antisense nucleic acid or a knock-out vector such that BEHAB expression and/or protein levels are thereby reduced in the recombinant cell), and the recombinant cell is administered to the recipient mammal. In this way, recombinant cells that express BEHAB at a reduced level can be administered to a mammal whose own cells express increased levels of BEHAB thereby treating or alleviating a disease, disorder or condition associated with or mediated by increased BEHAB expression as disclosed elsewhere herein.

V. Antibodies

Also included is an antibody that specifically binds BEHAB, a BEHAB cleavage product, or fragments thereof.

One skilled in the art would understand, based upon the disclosure provided herein, that an antibody that specifically binds BEHAB and/or a BEHAB cleavage product, binds with a BEHAB protein, or an immunogenic portion thereof, preferably the cleavage site discussed elsewhere herein. In one embodiment, the antibody is directed to mammalian BEHAB comprising the amino acid sequence SEQ ID NO:6.

The skilled artisan, when equipped with the present disclosure, would also understand that the present invention further comprises antibodies that bind a glycosylation-variant BEHAB isoform, including an underglycosylated BEHAB isoform and an unglycosylated BEHAB isoform. The generation of antibodies is described elsewhere herein, and their production is accomplished using techniques and skills well known in the art. Antibodies that bind glycosylation-variant BEHAB, including underglycosylated BEHAB and unglycosylated BEHAB include, but are not limited to the B5, B6 and B_{CRP} antibodies described in the experimental details herein and elsewhere in the art (Matthews et al., 2000, J. Biol. Chem. 275: 22695-22703). Further, the antibodies described herein can bind various forms of mammalian BEHAB, including rat and human, and art thus useful in the present invention for the detection, diagnosis, and treatment of primary CNS tumors associated with BEHAB.

The present invention is not limited to the antibodies enumerated herein, but rather also includes anti-glycosylation-variant BEHAB antibodies discovered and generated in the future. An antibody to a glycosylation-variant BEHAB, including a differently-glycosylated, underglycosylated and unglycosylated BEHAB, can be generated in a variety of ways well known in the art. As a non-limiting example, a nucleic acid encoding BEHAB, or a fragment thereof, can be transformed into an organism that does not glycosylate the proteins it produces, such as E. coli. Methods for the production of proteins in E. coli and other prokaryotic species are well known in the art and are described elsewhere herein. The protein isolated from a non-glycosylating prokaryotic species can then be administered to a mammal to generate antibodies, as is described herein. The antibodies specifically bind a glycosylation-variant BEHAB isoform, including unglycosylated BEHAB.

Further, antibodies to glycosylation-variant BEHAB can be generated by contacting a full-length BEHAB protein with glycosidases in order to remove some or all of the sugars and carbohydrates associated with the BEHAB protein backbone. Such glycosidases are well known in the art, and a number of relevant glycosidases are described elsewhere herein. Further, the skilled artisan, when equipped with the present disclosure and the data disclosed herein, would readily be able to select specific glycosidases for the removal of a certain family of sugars or carbohydrates while optionally retaining others on sugars and carbohydrates on the BEHAB molecule. The BEHAB molecule, after treatment with a glycosidase, can then be administered to an animal for the generation of antibodies to glycosylation-variant-BEHAB. Methods for the administration of a protein to a mammal and the generation of an antibody are well known in the art and are described herein.

The invention further comprises generating antibodies specific to glycosylation-variant BEHAB. Such antibodies are useful in the compositions, methods and kits disclosed elsewhere herein. As a non-limiting example, an antibody-specific to glycosylation-variant BEHAB can be generated by administering a peptide or protein comprising fragments of the primary amino acid sequence of BEHAB. Such fragments can comprise consensus glycosylation sites present in the primary amino acid sequence of BEHAB. The skilled artisan will readily recognize such consensus glycosylation sites by their sequences and amino acid content. As an example, O-linked saccharides are usually attached via a glycosidic bond on a threonine or serine residue, and in some cases, on hydroxylysine or hydroxyproline. Further, N-linked saccharides are often attached to an asparagine residue, often at a site having a sequence of any amino acid bound to an asparagine bound to any amino acid bound to threonine. Thus, the skilled routineer, when armed with the present disclosure and the methods disclosed herein, would readily be able to identify consensus glycosylation sites in a BEHAB primary amino acid sequence, generate peptides for immunizing an animal comprising these consensus glycosylation sites, and generate antibodies that specifically bind glycosylation-variant BEHAB. Such antibodies are useful in therapeutic treatments, including, but not limited to immunizing a mammal against the formation of primary CNS tumors, treating a primary CNS tumor, detecting a primary CNS tumor in a mammal either in vivo or in vitro, and other methods -and uses disclosed elsewhere herein.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the BEHAB portion is rendered immunogenic (e.g., BEHAB conjugated with keyhole limpet hemocyanin, KLH) and a portion comprising the respective rodent and/or human BEHAB amino acid residues. The chimeric proteins are produced by cloning the appropriate nucleic acids encoding BEHAB (e.g., SEQ ID NO:5 into a plasmid vector suitable for this purpose, such as but not limited to, pMAL-2 or pCMX. Other methods of producing antibodies that specifically bind BEHAB and portions thereof are detailed in Matthews et al. (2000, J. Biol. Chem. 275: 22695-22703).

However, the invention should not be construed as being limited solely to polyclonal antibodies that bind a full-length BEHAB. Rather, the invention should be construed to include other antibodies, as that term is defined elsewhere herein, to mammalian BEHAB, or portions thereof. Further, the present invention should be construed to encompass antibodies that, among other things, bind to BEHAB and are able to bind BEHAB present on Western blots, in immunohistochemical staining of tissues thereby localizing BEHAB in the tissues, and in immunofluorescence microscopy of a cell transiently or stably transfected with a nucleic acid encoding at least a portion of BEHAB.

One skilled in the art would appreciate, based upon the disclosure provided herein, that the antibody can specifically bind with any portion of the protein and the full-length protein can be used to generate antibodies specific therefor. However, the present invention is not limited to using the full-length protein as an immunogen. Rather, the present invention includes using an immunogenic portion of the protein to produce an antibody that specifically binds with mammalian BEHAB. That is, the invention includes immunizing an animal using an immunogenic portion, or antigenic determinant, of the BEHAB protein, for example, the epitope comprising the cleavage site, or a new antigenic site produced by proteolytic cleavage.

The antibodies can be produced by immunizing an animal such as, but not limited to, a rabbit or a mouse, with a BEHAB protein, or a portion thereof, or by immunizing an animal using a protein comprising at least a portion of BEHAB, or a fusion protein including a tag polypeptide portion comprising, for example, a maltose binding protein tag polypeptide portion, covalently linked with a portion comprising the appropriate BEHAB amino acid residues. One skilled in the art would appreciate, based upon the disclosure provided herein, that smaller fragments of these proteins can also be used to produce antibodies that specifically bind BEHAB.

One skilled in the art would appreciate, based upon the disclosure provided herein, that various portions of an isolated BEHAB polypeptide can be used to generate antibodies to either epitopes comprising the cleavage site of BEHAB or to epitopes present on the cleavage products of BEHAB. Once armed with the sequence of BEHAB and the detailed analysis localizing the various epitopes and cleavage products of the protein, the skilled artisan would understand, based upon the disclosure provided herein, how to obtain antibodies specific for the various portions of a mammalian BEHAB polypeptide using methods well-known in the art or to be developed.

Therefore, the skilled artisan would appreciate, based upon the disclosure provided herein, that the present invention encompasses antibodies that neutralize and/or inhibit BEHAB activity (e.g., by inhibiting necessary BEHAB cleavage product receptor/ligand interactions or BEHAB cleavage) which antibodies can recognize BEHAB or BEHAB cleavage products.

The invention should not be construed as being limited solely to the antibodies disclosed herein or to any particular immunogenic portion of the proteins of the invention. Rather, the invention should be construed to include other antibodies, as that term is defined elsewhere herein, to BEHAB, or portions thereof, or to proteins sharing at least about % homology with a polypeptide having the amino acid sequence of SEQ ID NO:6. Preferably, the polypeptide is about 1% homologous, more preferably, about 5% homologous, more preferably, about 10% homologous, even more preferably, about 20% homologous, more preferably, about 30% homologous, preferably, about 40% homologous, more preferably, about 50% homologous, even more preferably, about 60% homologous, more preferably, about 70% homologous, even more preferably, about 80% homologous, preferably, about 90% homologous, more preferably, about 95% homologous, even more preferably, about 99% homologous, and most preferably, about 99.9% homologous to BEHAB (SEQ ID NO:6).

One skilled in the art would appreciate, based upon the disclosure provided herein, that the antibodies can be used to localize the relevant protein in a cell and to study the role(s) of the antigen recognized thereby in cell processes. Moreover, the antibodies can be used to detect and or measure the amount of protein present in a biological sample using well-known methods such as, but not limited to, Western blotting and enzyme-linked immunosorbent assay (ELISA). Moreover, the antibodies can be used to immunoprecipitate and/or immuno-affinity purify their cognate antigen using methods well-known in the art. In addition, the antibody can be used to decrease the level of BEHAB or BEHAB cleavage products in a cell thereby inhibiting the effect(s) of BEHAB or BEHAB cleavage products in a cell. Thus, by administering the antibody to a cell or to the tissues of a mammal or to the mammal itself, the required BEHAB receptor/ligand interactions are therefore inhibited such that the effect of BEHAB cleavage is also inhibited. One skilled in the art would understand that inhibiting BEHAB cleavage using an anti-BEHAB antibody can include, but is not limited to, decreased tumor size, increased survival, and the like.

One skilled in the art would appreciate, based upon the disclosure provided herein, that the invention encompasses administering an antibody that specifically binds with BEHAB orally, parenterally, intraventricularly, intrathecally, intraparenchymally or by multiple routes, to inhibit BEHAB cleavage in the brain. Administration can include delivery by bioengineered polymers, direct injection, through an Ommaya reservoir (A device implanted under the scalp that is used to deliver anticancer drugs to the cerebrospinal fluid, or other such means well known to one of skill in the art of neurosurgery.

The invention encompasses polyclonal, monoclonal, synthetic antibodies, and the like. One skilled in the art would understand, based upon the disclosure provided herein, that the crucial feature of the antibody of the invention is that the antibody bind specifically with BEHAB. That is, the antibody of the invention recognizes BEHAB, or a fragment thereof (e.g., an immunogenic portion or antigenic determinant thereof), on Western blots, in immunostaining of cells, and immunoprecipitates BEHAB using standard methods well-known in the art.

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al. (1992, Critical Rev. Immunol. 12:125-168), and in the references cited therein, and in Gu et al. (1997, Thrombosis and Hematocyst 77:755-759). The present invention also includes the use of humanized antibodies specifically reactive with epitopes of BEHAB. Such antibodies are capable of specifically binding BEHAB, or a fragment thereof. The humanized antibodies of the invention have a human framework and have one or more complementarity determining regions (CDRs) from an antibody, typically, but not limited to a mouse antibody, specifically reactive with BEHAB, or a fragment thereof Thus, for example, humanized antibodies to BEHAB are useful in the treatment of primary CNS tumors such as gliomas, well-differentiated astrocytomas, anaplastic astrocytomas, glioblastoma multiforme, ependymomas, oligodendrogliomas, ganglioneuromas, mixed gliomas, brain stem gliomas, optic nerve gliomas, meningiomas, pineal tumors, pituitary tumors, pituitary adenomas, primitive neuroectodermal tumors, schwannomas, vascular tumors, lymphomas, and the like.

When the antibody used in the invention is humanized, the antibody may be generated as described in Queen, et al. (U.S. Pat. No. 6,180,370), Wright et al., (1992, Critical Rev. Immunol. 12:125-168) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as BEHAB, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO87/02671, which is herein incorporated by reference. CDRs useful in producing the antibodies of the present invention may be similarly derived from DNA encoding monoclonal antibodies capable of binding to BEHAB. Such humanized antibodies may be generated using well known methods in any convenient mammalian source capable of producing antibodies, including, but not limited to, mice, rats, rabbits, or other vertebrates. Suitable cells for constant region and framework DNA sequences and host cells in which the antibodies are expressed and secreted, can be obtained from a number of sources, for example, American Type Culture Collection, Manassas, Va.

In addition to the humanized antibodies discussed above, other modifications to native antibody sequences can be readily designed and manufactured utilizing various recombinant DNA techniques well known to those skilled in the art. Moreover, a variety of different human framework regions may be used singly or in combination as a basis for humanizing antibodies directed to BEHAB. In general, modifications of genes may be readily accomplished using a variety of well-known techniques, such as site-directed mutagenesis (Gillman and Smith, Gene, 8:81-97 (1979); Roberts et al., 1987, Nature, 328:731-734).

Alternatively, a phage antibody library may be generated. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor Laboratory, New York).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al. (992, Critical Rev. Immunol. 12:125-168).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CHI) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J. Mol. Biol. 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

VI. Compositions

The present invention encompasses a glycosylation-variant BEHAB isoform, including, but not limited to differently-glycosylated, underglycosylated BEHAB and unglycosylated BEHAB. The glycosylation-variant BEHAB of the present invention comprises a BEHAB molecule with altered or less than the full complement of sugars and carbohydrates found on full-length BEHAB. As disclosed by the data herein, glycosylation-variant BEHAB is the major upregulated form of BEHAB in primary CNS tumors, including, but not limited to, gliomas. Thus the present invention includes a glycosylation-variant BEHAB that is useful for, inter alia, a diagnostic tool for primary CNS tumors, a research tool for elucidating the interaction of the neural extracellular matrix with cancer-causing mutations, dysfunctions, and the like. Further, the glycosylation-variant BEHAB of the present invention is useful as a reagent in compositions, methods and kits for the detection, treatment, and diagnosis of primary CNS tumors.

Glycosylation-variant BEHAB can be made according to the methods disclosed herein. That is, the present invention comprises methods for the isolation of glycosylation-variant BEHAB from the particulate fraction of brain homogenate, and further includes methods for the differentiation of glycosylation-variant BEHAB from other BEHAB molecules, including full-length BEHAB and GPI-linked BEHAB.

The present invention further comprises methods for the generation of glycosylation-variant BEHAB in a recombinant cell. That is, the skilled artisan, when equipped with the present disclosure and the data herein, can produce glycosylation-variant BEHAB by transfecting a cell with an isolated nucleic acid encoding BEHAB, or a fragment thereof, and isolating glycosylation-variant BEHAB from a cell. Isolated nucleic acids for this purpose are disclosed elsewhere herein, as are methods for the transfection and expression of a protein in a cell. Preferably, the cell is a cell that expresses glycosylation-variant BEHAB, such as, but not limited to, and Oli-neu cell.

As described by the data disclosed herein, a glycosylation-variant BEHAB can be differentiated from full-length BEHAB or GPI-anchored BEHAB through various methods. Such methods include SDS-PAGE electrophoresis, immunofluorescence and localization, immunoprecipitation, and the like. Further, the skilled artisan would readily be able to distinguish between a different isoform of a protein based on glycosylation using techniques known in the art and described herein.

VII. Methods

A. Methods of Treating a Primary CNS Tumor

The present invention is based, in part, on the novel discovery that BEHAB plays a significant role in primary CNS tumor progression, invasiveness and the survival time of mammals with brain tumors. As demonstrated by the data disclosed herein, BEHAB cleavage potentiates the progression of primary CNS tumors, and inhibition of cleavage, and/or inhibition of the function of BEHAB and its cleavage products can be used as a treatment for a primary CNS tumor in a mammal. In all instances, whether treating or diagnosing a primary CNS tumor, the most preferred mammal is a human.

The present invention includes a method of treating a primary CNS tumor in a mammal, preferably a human. This is because, as demonstrated by the data disclosed elsewhere herein, cleavage of BEHAB, and/or the function, biological activity and expression of BEHAB cleavage products is critical to the progression and invasiveness of primary CNS tumors. Therefore, as is evident from the data presented herein, inhibiting the cleavage of BEHAB, and/or inhibiting the function, biological activity, and expression of BEHAB cleavage products can serve as a treatment for primary CNS tumors. One skilled in the art would appreciate, based on the present disclosure, that inhibiting the cleavage of BEHAB provides an important and novel therapeutic for the treatment of among other things, gliomas, well-differentiated astrocytomas, anaplastic astrocytomas, glioblastoma multiforme, ependymomas, oligodendrogliomas, ganglioneuromas, mixed gliomas, brain stem gliomas, optic nerve gliomas, meningiomas, pineal tumors, pituitary tumors, pituitary adenomas, primitive neuroectodermal tumors, schwannomas, vascular tumors, lymphomas, reactive gliosis, and the like. One of skill in the art will also recognize that, like primary CNS tumors, glial cell activation and proliferation arises from injury to the CNS, i.e. the brain and spinal cord. Such glial cell activation and proliferation that result from these injuries are well known in the art and often referred to as reactive gliosis. Reactive gliosis is detailed in, for example, Streit (2000, Toxicol. Pathol., 28:28-30). The present invention includes methods for treating reactive gliosis in a mammal, preferably a human.

An inhibitor of BEHAB cleavage is administered to a mammal, thereby decreasing BEHAB cleavage and providing a therapeutic benefit. The skilled artisan would appreciate, based upon the disclosure provided herein, that BEHAB cleavage can be inhibited using a wide range of techniques known or to be developed in the future. That is, the invention encompasses inhibiting the cleavage of BEHAB in a mammal, and thereby preventing the progression and invasiveness of a primary CNS tumor. The present invention discloses methods for inhibiting BEHAB cleavage in a mammal, e.g. blocking the cleavage site, titrating the protease responsible for cleavage, and expressing or administering a non-cleavable BEHAB mutant. This is because, as demonstrated by the data disclosed herein, affecting the cleavage of BEHAB mediates a variety of effects, including, but not limited to decreased tumor size and increased survival time in mammals afflicted with primary CNS tumors, and thereby provides a novel and powerful therapeutic for primary CNS tumors.

The skilled artisan will further understand when equipped with this disclosure and the data presented herein, that administering to a mammal an inhibitor of the function, biological activity, and expression of BEHAB and/or its cleavage products provides a beneficial therapeutic to a mammal with a primary CNS tumor. The present invention includes methods for reducing or preventing the expression of BEHAB and binding the cleavage products and/or their ligands. As demonstrated by the data disclosed herein, increased levels of BEHAB and the biological activity of BEHAB cleavage products mediate enhanced progression of primary CNS tumors, resulting in decreased survival rates and larger tumors. Therefore, a method for inhibiting BEHAB expression or BEHAB cleavage product expression and/or function is included in the present invention.

The skilled artisan would understand that inhibiting BEHAB cleavage encompasses blocking the cleavage site, titrating the protease responsible for cleavage, and expressing and/or administering a non-cleavable BEHAB mutant. The present invention includes a method for inhibiting the cleavage of BEHAB by blocking the cleavage site on the protein. As disclosed herein, the cleavage site comprises Glu³⁹⁵-Ser³⁹⁶ of the BEHAB protein. Therefore, inaccessibility of this cleavage site to a protease can prevent the cleavage of BEHAB. The present invention therefore includes methods for inhibiting the cleavage of BEHAB by blocking access to the cleavage site by proteases. As an example, an antibody or other ligand to a portion of the protein comprising the cleavage site, or a peptide or a small molecule that interacts with the cleavage site, would block access to the protein by a protease, thereby inhibiting BEHAB cleavage. The skilled artisan would appreciate, when armed with the disclosure and data disclosed herein, that an antibody can specifically bind a short peptide comprising the cleavage site, or to a larger portion of the BEHAB protein, provided that the antibody or the ligand blocks the cleavage site.

Methods of generating antibodies to BEHAB are well known in the art (Matthews et al., 2000, J. Biol. Chem. 275: 22695-22703) and are disclosed elsewhere herein. Further, methods for producing antibodies that specifically bind certain epitopes of a protein are well known in the art and can be accomplished using standard methods disclosed herein and elsewhere, see Harlow et al. (1988, Antibodies: A Laboratory Manual, Cold Spring Harbor, N.Y.).

One of skill in the art will appreciate that an antibody can be administered as a protein, a nucleic acid construct encoding a protein, or both. Numerous vectors and other compositions and methods disclosed elsewhere herein are well known for administering a protein or a nucleic acid construct encoding a protein to cells or tissues. Therefore, the invention includes a method of administering an antibody or nucleic acid encoding an antibody (synthetic antibody) that is specific for BEHAB (Sambrook et al., 1989, Molecular Cloning: A Laboratory Manual, Cold Spring Harbor Laboratory, New York; Ausubel et al., 1997, Current Protocols in Molecular Biology, John Wiley & Sons, New York).

One skilled in the art would understand, based upon the disclosure provided herein, that an antibody can be administered such that it blocks the cleavage site on BEHAB present in a mammal. Moreover, the invention encompasses administering an antibody that specifically binds with BEHAB, or a nucleic acid encoding the antibody, wherein the molecule further comprises an intracellular retention sequence such that the antibody binds with BEHAB and prevents its GPI-anchored expression or secretion. Such antibodies, frequently referred to as “intrabodies”, are well known in the art and are described in, for example, Marasco et al. (U.S. Pat. No. 6,004,490) and Beerli et al. (1996, Breast Cancer Research and Treatment 38:11-17). Thus, the invention encompasses methods comprising inhibiting BEHAB cleavage where BEHAB is present in a mammal, as well as methods of inhibiting BEHAB cleavage comprising inhibiting BEHAB being present in its GPI-anchored on a cell membrane form or its secreted, and such methods as become known in the future.

The present invention further comprises a method of treating a primary CNS tumor or reactive gliosis in a mammal, including a human, by administering to the mammal an effective amount of glycosylation-variant BEHAB isoform inhibitor. That is, the present invention encompasses a method for treating a primary CNS tumor in a mammal, including, gliomas, well-differentiated astrocytomas, anaplastic astrocytomas, glioblastoma multiforme, ependymomas, oligodendrogliomas, ganglioneuromas, mixed gliomas, brain stem gliomas, optic nerve gliomas, meningiomas, pineal tumors, pituitary tumors, pituitary adenomas, primitive neuroectodermal tumors, schwannomas, vascular tumors, lymphomas, and the like. The method comprises administering an antibody to a mammal wherein the antibody or other ligand binds to a glycosylation-variant BEHAB isoform and thus treats a primary CNS tumor. This is because, as demonstrated by the data disclosed herein, glycosylation-variant BEHAB is the major isoform of BEHAB present in primary CNS tumors, including gliomas and the like. Therefore, the present invention is useful in inhibiting the activity of a glycosylation-variant BEHAB in the CNS and thus treating a primary CNS tumor.

Methods for the generation and administration of an antibody that specifically binds a glycosylation-variant BEHAB isoform are well known in the art and are described elsewhere herein. The present invention further comprises intrabodies, antibodies administered as a protein, and antibodies administered as a nucleic acid construct encoding an antibody that binds a glycosylation-variant BEHAB isoform, including an underglycosylated BEHAB isoform and an unglycosylated BEHAB isoform.

The present invention also encompasses methods for inhibiting BEHAB cleavage by inhibiting the protease responsible for BEHAB cleavage. This is because, as is evident from the data presented herein, BEHAB is cleaved by a protease at a specific site, but inhibiting the cleavage of BEHAB results in, among other things, smaller tumor volumes and increased animal survival rates. Therefore, the present invention includes a method of inhibiting BEHAB cleavage by inhibiting the protease that cleaves BEHAB.

One of skill in the art will recognize that inhibiting a protease comprises administering to a mammal an effective amount of a protease inhibitor. Such inhibitors include, but are not limited to, chemical compounds, including tissue inhibitor of metalloproteinases 2, tissue inhibitor of metalloproteinases 3, inhibitors of ADAMTS proteases, small molecules, an antibody or other molecule that specifically binds a protease that cleaves BEHAB, and the like. Specific protease inhibitors are well known in the art, and are discussed in, for example, Martel-Pelletier et al., (2001, Best Pract. Res. Clin. Rheumatol. 15:805-29). The skilled artisan, when armed with the present disclosure and teachings herein, will readily understand how to administer a protease inhibitor to a mammal, and therefore, the present invention encompasses protease inhibitors as a treatment for primary CNS tumors.

The present invention also encompasses methods for inhibiting BEHAB cleavage by titrating the protease responsible for BEHAB cleavage. This is because, as is evident from the data presented herein, BEHAB is cleaved by a protease at a specific site, but the mutant BEHAB of the present invention cannot be cleaved by a protease, as measured in both in vivo and in vitro assays. Further, the protease that cleaves BEHAB is present in the body in limited amounts and limited locations compared to other metalloproteinases. Therefore, an uncleavable BEHAB is capable of titrating the protease so it is not available to cleave endogenous BEHAB. One of skill in the art will recognize that titrating a protease encompasses providing a substrate that reduces the functional concentration of the protease in a mammal, preferably a human, that is available to cleave BEHAB. Titrating a protease further includes providing a substrate that is recognized and bound by a protease, resulting in a decline in the number of proteases or protease active sites available to cleave BEHAB.

As described more fully elsewhere herein, a tumor expressing a mutant, uncleavable form of BEHAB, even in the presence of endogenous BEHAB, results in among other things, smaller tumor volumes and increase survival rates in animals. These data indicate that even though endogenous BEHAB is present in a cell, the additional presence of an uncleavable BEHAB results in the decreased progression of a primary CNS tumor in an art accepted in vivo primary CNS tumor model. The data further indicate that when tumors expressing exogenous mutant BEHAB are compared to tumors expressing exogenous full-length BEHAB, tumors expressing mutant BEHAB are both smaller and result in longer animal survival times. While not wishing to be bound by any particular theory, the data presented herein indicate that an uncleavable BEHAB mutant titrates the protease responsible for BEHAB cleavage, and as a result of decreased cleavage, decreased tumor progression ensues.

The skilled artisan would appreciate, based on the present disclosure and the data disclosed herein that a non-cleavable substrate for the protease inhibits tumor progression by decreasing tumor size and increasing survival rates in animals afflicted with primary CNS tumors. Therefore, the present invention includes a method for treating a primary CNS tumor by titrating the protease that cleaves BEHAB.

Compounds used to titrate the protease that cleaves BEHAB include, but are not limited to, peptides, proteins, mimetopes and peptidomimetics. As disclosed elsewhere herein, non-cleavable BEHAB (mutant BEHAB, SEQ ID NO:3) comprises the native BEHAB protein with a mutation in the amino acid sequence surrounding the cleavage site, specifically a mutation of Glu-Ser-Glu-Ser-Arg-Gly to Glu-Ser-Glu-Asn-Val-Tyr (SEQ ID NO:1 and SEQ ID NO:2, respectively). One of skill in the art will readily appreciate that a peptide derived from full length mutant BEHAB can exhibit the same protease titrating properties as the full length mutant BEHAB protein set forth in SEQ ID NO:3. Thereby the present invention encompasses the full length mutant BEHAB protein and truncated mutant BEHAB peptides comprising protease titrating activity.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The peptides of the present invention may be readily prepared by standard, well-established solid-phase peptide synthesis (SPPS) as described by Stewart et al. (Solid Phase Peptide Synthesis, 2nd Edition, 1984, Pierce Chemical Company, Rockford, Ill.) and as described by Bodanszky and Bodanszky (The Practice of Peptide Synthesis, 1984, Springer-Verlag, New York). At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF (hydrofluoric acid) treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequencers, which sequentially degrade the peptide and identify the amino acids in order, may also be used to determine definitely the sequence of the peptide.

Prior to its use, the peptide is purified to remove contaminants. In this regard, it will be appreciated that the peptide will be purified so as to meet the standards set out by the appropriate regulatory agencies or for specific uses. Any one of a number of a conventional purification procedures may be used to attain the required level of purity including, for example, reversed-phase high-pressure liquid chromatography (HPLC) using an alkylated silica column such as C₄-,C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

One of skill in the art would readily appreciate that a mutant BEHAB protein or peptide capable of titrating a protease that cleaves BEHAB may be administered to a mammal as an isolated nucleic acid encoding a mutant BEHAB protein or peptide. Methods of expressing a desired protein in a cell or a mammal are well known in the art, and when combined with the present disclosure and the data herein, the skilled artisan will to be able to express a mutant BEHAB protein or peptide in a cell or a mammal without undue experimentation.

One of skill in the art will appreciate that many methods exist for the expression of a protein or peptide in a cell or a mammal, including the introduction of a vector or expression vector comprising an isolated nucleic acid encoding the desired protein or peptide into a cell or mammal. The skilled artisan will further appreciate that a vector can comprise the isolated nucleic of SEQ ID NO:4, or some biologically active portion thereof.

The present invention also includes mimetopes of a mutant BEHAB protein and peptide of the present invention. As used herein, a mimetope of a mutant BEHAB protein or peptide refers to any compound that is able to mimic the activity of such a mutant BEHAB protein or peptide (e.g., ability to titrate a protease that cleaves BEHAB, thereby preventing the cleavage of native BEHAB), often because the mimetope has a structure that mimics the mutant BEHAB protein or peptide. It is to be noted, however, that the mimetope need not have a structure similar to an mutant BEHAB protein or peptide as long as the mimetope functionally mimics the protein. Mimetopes can be, but are not limited to: peptides that have been modified to decrease their susceptibility to degradation; anti-idiotypic and/or catalytic antibodies, or fragments thereof; non-proteinaceous immunogenic portions of an isolated protein (e.g., carbohydrate structures); synthetic or natural organic or inorganic molecules, including nucleic acids; and/or any other peptidomimetic compounds. Mimetopes of the present invention can be designed using computer-generated structures of a mutant BEHAB protein or peptide of the present invention. Mimetopes can also be obtained by generating random samples of molecules, such as oligonucleotides, peptides or other organic molecules, and screening such samples by affinity chromatography techniques using the corresponding binding partner, (e.g., a protease that cleaves BEHAB or anti-BEHAB antibody). A preferred mimetope is a peptidomimetic compound that is structurally and/or functionally similar to a mutant BEHAB protein or peptide of the present invention, particularly to the cleavage site of the mutant BEHAB protein. Methods for generating mimetopes and peptidomimetics are well known in the art, and are detailed in, for example, Kazmierski (1999, Peptidomimetics Protocols (Methods in Molecular Medicine Vol. 23) Humana Press, Totowa N.J.).

The present invention also includes methods for inhibiting the expression and/or activity of BEHAB in a mammal. The skilled artisan will understand, when equipped with the present disclosure and the data disclosed herein, that higher levels of BEHAB expression increase tumor size and decrease survival rates in mammals afflicted with primary CNS tumors. That is, the data presented elsewhere herein demonstrate, for the first time, that mammals with primary CNS tumors overexpressing BEHAB have larger tumor volumes and shorter survival times when compared to mammals expressing normal levels of BEHAB, or to mammals expressing mutant BEHAB. Thus, the skilled artisan will certainly appreciate that a method of treating a primary CNS tumor encompasses inhibiting BEHAB expression.

An inhibitor of BEHAB expression and/or activity is administered to a mammal thereby decreasing BEHAB and providing a therapeutic benefit. The skilled artisan would appreciate, based upon the disclosure provided herein, that BEHAB can be inhibited using a wide plethora of techniques well-known in the art or to be developed in the future. That is, the invention encompasses inhibiting BEHAB expression, e.g., inhibition of transcription and/or translation. This is because, as demonstrated by the data disclosed elsewhere herein, reduced levels of BEHAB expression and/ or activity mediated a variety of effects, including, but not limited to, decreased tumor size and increased survival rates. Thus, inhibiting BEHAB includes, but is not limited to, inhibiting translation and/or transcription of a nucleic acid encoding the protein.

Further, the routineer would understand, based upon the disclosure provided elsewhere herein, that inhibition of BEHAB includes, but is not limited to, inhibiting the biological activity of the molecule. This is because, as the data disclosed elsewhere herein demonstrate, inhibition of BEHAB activity, in that BEHAB is not cleaved by an endogenous protease, limits the progression of a primary CNS tumor. These data indicate that inhibition of BEHAB activity provides a therapeutic benefit for treatment of a disease, such as, but not limited to, primary CNS tumors, and the like.

The present invention encompasses inhibiting BEHAB by inhibiting expression of a nucleic acid encoding BEHAB. Methods for inhibiting the expression of a gene are well known to those of ordinary skill in the art, and include the use of ribozymes or antisense nucleic acid molecules.

Antisense nucleic acid molecules are DNA or RNA molecules that are complementary to some portion of an mRNA molecule. When present in a cell, antisense nucleic acids hybridize to an existing mRNA molecule and inhibit translation into a gene product. Inhibiting the expression of a gene using an antisense nucleic acid molecule is well known in the art (Marcus-Sekura, 1988, Anal. Biochem. 172:289), as are methods to express an antisense nucleic acid molecule in a cell (Inoue, 1993, U.S. Pat. No. 5,190,931).

The invention encompasses inhibiting the expression of BEHAB using a ribozyme. Using ribozymes for inhibiting gene expression is well known to those of ordinary skill in the art (Cech et al., 1992, J. Biol. Chem. 267:17479-17482; Hampel et al., 1989, Biochemistry 28: 4929-4933; Altman et al., 1992, U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030-3034), allowing the selective cleavage of specific mRNA molecules. Given the nucleotide sequence of BEHAB is well known in the art (Hockfield et al., 1997, U.S. Pat. No. 5,635,370) one of ordinary skill in the art can synthesize an antisense polynucleotide or ribozyme without undue experimentation, provided with the disclosure and references incorporated herein.

The skilled artisan will further appreciate, when armed with the present disclosure and the data presented herein, that cleavage of BEHAB mediates progression of primary CNS tumors. While not wishing to be bound by any particular theory, it can be theorized that while BEHAB is normally expressed endogenously at low levels and does not necessarily cause primary CNS tumors during normal expression, the cleavage of BEHAB, or more specifically the products of the cleavage event mediate the progression of a primary CNS tumor in a mammal. Thereby, as will be recognized by one of skill in the art, inhibiting the activity of the BEHAB cleavage products can be used as a method of treating a mammal afflicted with a primary CNS tumor.

The invention also encompasses the use of pharmaceutical compositions of an appropriate antibody, protein or peptide, mimetope, peptidomimetic, and/or isolated nucleic acid to practice the methods of the invention, the compositions comprising an appropriate antibody, protein or peptide, mimetope, peptidomimetic, and/or isolated nucleic acid and a pharmaceutically-acceptable carrier.

As used herein, the term “pharmaceutically-acceptable carrier” means a chemical composition with which an appropriate antibody, protein or peptide, mimetope, peptidomimetic, and/or isolated nucleic acid may be combined and which, following the combination, can be used to administer the appropriate antibody, protein or peptide, mimetope, peptidomimetic, and/or isolated nucleic acid to a mammal.

The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal.

Pharmaceutical compositions that are useful in the methods of the invention may be administered systemically in oral solid formulations, ophthalmic, suppository, aerosol, topical or other similar formulations. They can be administered directly into the CNS intrathecally, intraventricularly, intraparenchymally, via direct injection, or via bioengineered polymers. In addition to the appropriate antibody, protein or peptide, mimetope, peptidomimetic, and/or isolated nucleic acid, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate hypericin derivative according to the methods of the invention.

Compounds which are identified using any of the methods described herein may be formulated and administered to a mammal for treatment of the diseases disclosed herein are now described.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to mammals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, rodents (including rats and mice), birds including commercially relevant birds such as chickens, ducks, geese, and turkeys.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal, intraventricular, intraparenchymal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e. about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 650 F at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution, suspension, or slow-release polymer. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Another formulation is the activate ingredient incorporated in a slow-release polymer. Such polymers are well known in the pharmaceutical arts, and are detailed in, for example, U.S. Pat. Nos. (4,728,512; 4,728,513; 5,084,287; 5,285,186).

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

A pharmaceutical composition of the invention can be prepared, packaged, or sold in a formulation suitable for direct CNS administration. Such formulations may, for example, be in the form of liquid administered by an Ommaya reservoir, by intrathecal or intraventricular administration, by direct intraparenchymal injection, by slow-release polymers, or other such methods well known in the pharmaceutical and neurological fields.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to a mammal, preferably a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to a mammal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the animal, etc.

The present invention further comprises a method of treating a primary CNS tumor using immune therapy. That is, the present invention includes a method of treating a primary CNS tumor by modulating the immune system in order to facilitate the recognition of a tumor expressing glycosylation-variant BEHAB by immune effector cells, thereby destroying the tumor. This is because, as demonstrated by the data disclosed herein, glycosylation-variant BEHAB is expressed exclusively on all human glioma samples assayed, and is not expressed on healthy human brain tissue. Thus, the present invention provides an immune therapy mediated method for the treatment of primary CNS tumors without harming healthy tissue.

Immune therapy can comprise the delivery of agents with established tumor-immune reactivity, such as effector cells, that can directly or indirectly mediate anti-tumor effects. Examples of effector cells include T cells, T lymphocytes (such as CD8+ cytotoxic T lymphocytes and CD4+ T-helper tumor-infiltrating lymphocytes), killer cells (such as natural killer cells and lymphokine-activated killer cells), B cells and antigen-presenting cells (such as dendritic cells and macrophages) expressing a glycosylation-variant BEHAB. T cell receptors and antibody receptors specific for a glycosylation-variant BEHAB may be cloned, expressed and transferred into other vectors or effector cells for adoptive immune therapy.

Effector cells may generally be obtained from a patient using techniques well known in the art, and expanded in vitro until sufficient quantities are available for immune therapy. Culture conditions for expanding antigen-specific effector cells to several billion in number with retention of antigen recognition in vivo are well known in the art. Such in vitro culture conditions would typically comprise intermittent stimulation with glycosylation-variant BEHAB, often in the presence of cytokines (such as IL-2) and non-dividing feeder cells. In particular, antigen-presenting cells, such as dendritic, macrophage, monocyte, fibroblast and/or B cells, can be pulsed with glycosylation-variant BEHAB and expanded for ex vivo immune therapy. Methods for obtaining and culturing antigen-specific immune effector cells are well known in the art, and are described in, for example, Cheever et al., (1997, Imm. Rev. 157: 177-194).

Alternatively, a vector expressing a BEHAB protein can be introduced into antigen presenting cells, such as dendritic cells, taken from a patient and clonally propagated ex vivo for transplant back into the same patient. Transfected cells may be reintroduced into the patient using any means known in the art, preferably in sterile form by intravenous, intracavitary, intraperitoneal or intratumor administration. Methods for the transfection, expansion, and reintroduction of immune effector cells are well known in the art, and are described in, for example, U.S. Pat. No. 6,500,641 and Cheever et al., (1997, Imm. Rev. 157: 177-194).

Routes and frequency of administration of the immune therapies described herein, as well as dosage, will vary from individual to individual, and may be readily established using standard techniques well known in the art. The response to a primary CNS tumor immune therapy can be monitored by measuring the anti-tumor antibodies in a patient or by the generation of cytolytic effector cells capable of killing the patient's tumor cells in vitro after administration of an immune therapy. Such immune therapies should also be capable of causing an immune response that leads to an improved clinical outcome (e.g., more frequent remissions, complete or partial or longer disease-free survival) in treated patients as compared to untreated patients.

B. Methods of Diagnosing a Primary CNS Tumor

The present invention further encompasses methods for the diagnosis of primary CNS tumors, other central nervous system tumors, and other neuropathological disorders relating to BEHAB, including, but not limited to, gliomas, well-differentiated astrocytomas, anaplastic astrocytomas, glioblastoma multiforme, ependymomas, oligodendrogliomas, ganglioneuromas, mixed gliomas, brain stem gliomas, optic nerve gliomas, meningiomas, pineal tumors, pituitary tumors, pituitary adenomas, primitive neuroectodermal tumors, schwannomas, vascular tumors, lymphomas, and the like. This is because, as demonstrated by the data disclosed elsewhere herein, BEHAB overexpression is highly correlated with the progression and invasiveness of primary CNS tumors and the like. The present invention therefore includes methods of determining the level of expression of BEHAB in a mammal, and therefore a method of diagnosing a primary CNS tumor. In all instances recited herein, whether treating or diagnosing a primary CNS tumor, the most preferred mammal is a human.

The invention includes a method of diagnosing a primary CNS tumor in a mammal. The method comprises obtaining a biological sample from a first mammal and comparing the level of BEHAB (expression, amount, activity) in that sample with the level of BEHAB in a sample obtained from a normal second mammal that is otherwise identical to the first mammal but which is not afflicted with a primary CNS tumor. A higher level of BEHAB in the sample from the first mammal compared with the level of BEHAB in the sample obtained from the second otherwise identical mammal not afflicted with a primary CNS tumor is an indication that the first mammal is afflicted with a primary CNS tumor. This is because, as disclosed elsewhere herein, an increased level of BEHAB expression is associated with, inter alia, larger tumor volumes and decreased survival rates.

The invention also encompasses a method of diagnosing a primary CNS tumor in a mammal, including a human, in vivo or in vitro. That is, the present invention includes a method of diagnosing a primary CNS tumor either in a mammal or in a biological sample from a mammal. The method comprises assessing and comparing the level of glycosylation-variant BEHAB in a mammal suspected of having a primary CNS tumor with the level of glycosylation-variant BEHAB in a mammal not suspected of having a primary CNS tumor. A higher level of glycosylation-variant BEHAB in a mammal suspected of having a primary CNS tumor when compared to a mammal not suspected of having a primary CNS tumor is a indication that the animal suspected of having a primary CNS tumor actually has a primary CNS tumor, thereby diagnosing a primary CNS tumor.

Comparing the level of glycosylation-variant BEHAB in a biological sample can be accomplished using any of the methods disclosed herein or known in the art, including detection with an antibody, such as ELISA, immunoblotting techniques, protein detection techniques, such as SDS-PAGE electrophoresis, nucleic acid techniques such as PCR and LCR, nucleic acid hybridization techniques, including Southern blotting, Northern blotting, and other techniques well known in the art. As an example, a biological sample can be obtained from a mammal, and assessed for the level of glycosylation variant BEHAB in that sample. The biological sample can include, but is not limited to, blood, urine, feces, neural tissue, cerebrospinal fluid, saliva, brain tissue, and the like. The biological sample can be obtained by various methods depending on the biological sample to be obtained. For example, blood can be obtained through venipuncture; urine, feces, and saliva can be captured in a specimen vessel and the like. Tissue samples, including, but not limited to brain tissue and neural tissue can be obtained through a biopsy or similar methods well known in the art. Cerebrospinal fluid can be collected through a spinal tap using methods well known in the art.

For the in vivo detection of glycosylation-variant BEHAB or the diagnosis of a primary CNS tumor related to glycosylation variant BEHAB, the skilled artisan can employ a tagged antibody or nucleic acid for the detection of glycosylation-variant BEHAB in a mammal. Such antibodies can be generated using techniques described elsewhere herein and then conjugated to a tag or other molecule capable of detection through a number of methods. Methods of conjugating a tag or other molecule to an antibody are well known in the art and can be accomplished using techniques in protein chemistry, described elsewhere herein. As an example, an antibody that binds glycosylation-variant BEHAB can be conjugated to a radioactive isotope and the binding of the isotope tagged antibody can be detected on a film sensitive to radioactivity, such as X-ray film. The antibody can also be bound to a tag visible to magnetic resonance imaging technology. Further, the present invention includes a method in which an antibody is conjugated to fluorescent molecule, such as luciferase or green fluorescent protein, or another tag, such as horseradish-peroxidase, a fluorescent molecule, an enzyme, gold, biotin, a radioactive isotope, or gadolinium, and the binding of the antibody to a glycosylation-variant BEHAB isoform is detected through an imaging system capable of visualizing a tag. Uses of biophotonic imaging systems for the in vivo detection of fluorescent tags are well known in the art and such systems are available commercially (Xenogen, Alameda, Calif.). The invention further includes a method of diagnosing primary CNS tumor progression in a mammal. As will be appreciated by the skilled artisan, once armed with the present disclosure and the data herein, BEHAB cleavage mediates the progression of brain tumors, resulting in, among other things, larger tumor volumes and decreased survival times. Therefore, the present invention includes a method of diagnosing brain tumor progression in a mammal. The method comprises obtaining a biological sample from a first mammal and comparing the level of BEHAB cleavage in that sample with the level of BEHAB cleavage in a sample obtained from a normal second mammal that is otherwise identical to the first mammal but which is not afflicted with a primary CNS tumor, or is afflicted with a primary CNS tumor that has not progressed as far as the primary CNS tumor in the second mammal, as can be easily determined by one of skill in the art using standard neurological indicators. A higher level of BEHAB cleavage in the sample from the first mammal compared with the level of BEHAB cleavage in the sample obtained from the second otherwise identical mammal is an indication that the first mammal is afflicted with a primary CNS tumor progressing at a higher rate. This is because, as disclosed elsewhere herein, an increased level of BEHAB cleavage is associated with larger tumor volumes and decreased survival rates, and the like.

One of skill in the art will appreciate, when armed with the present disclosure and data herein, that methods for determining the level of BEHAB cleavage include, but are not limited to Western blotting, ELISA, and other immuno-detection assays well known in the art.

In one aspect, the biological sample is selected from the group consisting of a blood sample, a neurological tissue biopsy, a cerebrospinal fluid sample, urine, saliva, and the like.

The invention includes a method of assessing the effectiveness of a treatment for a primary CNS tumor in a mammal. The method comprises assessing the level of BEHAB expression, amount, and/or activity, before, during and after a specified course of treatment for a disease, disorder or condition mediated by or associated with increased BEHAB expression (e.g., primary CNS tumors and the like). This is because, as stated previously elsewhere herein, increased BEHAB expression, amount and/or activity is associated with or mediates larger tumor volumes and decreased animal survival rates, which is a feature of increased mortality due to primary CNS tumors.

Thus, assessing the effect of a course of treatment upon BEHAB expression/amount/activity indicates the efficacy of the treatment such that a lower level of BEHAB expression, amount, or activity indicates that the treatment method is successful.

The course of therapy to be assessed can include, but is not limited to, surgery, chemotherapy, radiation therapy, and/or the multiple modes of therapy for a primary CNS tumor disclosed herein.

The invention also includes a method for assessing the effectiveness of a treatment for a primary CNS tumor in a mammal. The method comprises assessing the level of glycosylation-variant BEHAB in a mammal before, during, or after administration a treatment for a primary CNS tumor. As an example, a biological sample from a mammal is obtained before the administration of a therapy for a primary CNS tumor, and other samples are obtained during and after therapy for a primary CNS tumor has been administered. Collection of the biological sample is accomplished according to methods well known in the art and described elsewhere herein. The level of glycosylation-variant BEHAB is assessed according to the methods disclosed elsewhere herein. The level of glycosylation-variant BEHAB, including underglycosylated BEHAB and unglycosylated BEHAB is compared between the biological samples taken before, during and after a therapy for a primary CNS tumor, providing an indication of the effectiveness of the treatment for a primary CNS tumor.

The invention also includes assessing the effectiveness of a treatment for a primary CNS tumor in a mammal before, during and after treatment of a primary CNS tumor. The effectiveness of a therapy for a primary CNS tumor can be assessed in a mammal using techniques disclosed elsewhere herein and well known in the art. That is, the present invention includes a method of assessing the effectiveness of a treatment for a primary CNS tumor by comparing the level of a glycosylation-variant BEHAB isoform in vivo using methods disclosed elsewhere herein.

The invention encompasses probes and primers for detecting the expression, amount, or activity of a BEHAB gene. The skilled artisan, when equipped with the present disclosure and the data disclosed herein, will appreciate that probes are provided that are capable of specifically hybridizing to DNA or RNA of a BEHAB gene. For purposes of the present invention, probes are “capable of hybridizing” to DNA or RNA of BEHAB if they hybridize to a BEHAB gene under conditions of either high or moderate stringency, see Sambrook et al. (1989, Molecular Cloning: A Laboratory Manual, Cold Spring Harbor Laboratory, New York) but not significantly or detectably to an unrelated gene. Preferably, the probe hybridizes to suitable nucleotide sequences under high stringency conditions, such as hybridization in 5×SSPE, 1×Denhardt's solution, 0.1% SDS at 65° C., and at least one wash to remove unhybridized probe in the presence of 0.2×SSC, 1×Denhardt's solution, 0.1% SDS at 65° C. Except as otherwise provided herein, probe sequences are designed to allow hybridization to a BEHAB gene, but not to DNA or RNA sequences from other genes. The probes are used, for example, to hybridize to nucleic acid that is present in a biological sample, including, but not limited to, blood, cerebrospinal fluid, lymph, or tissue, isolated from a patient. The hybridized probe is then detected, thereby indicating the presence of the desired cellular nucleic acid. The skilled artisan will recognize that the cellular nucleic acid can be subjected to an amplification procedure, such as polymerase chain reaction (PCR), prior to hybridization. Alternatively, a BEHAB gene can be amplified and the amplified product subjected to DNA sequencing. A BEHAB gene can be detected by DNA sequence analysis or hybridization with a BEHAB specific oligonucleotide probe under conditions and for a time sufficient to allow hybridization to the specific allele. Typically, the hybridization buffer can contain tetramethyl ammonium chloride and the like, see Sambrook et al. (1989, Molecular Cloning: A Laboratory Manual, Cold Spring Harbor Laboratory, New York).

Nucleic acid probes of the present invention may be composed of either deoxyribonucleic acids (DNA), ribonucleic acids (RNA), nucleic acid analogues (e.g., peptide nucleic acids), or any combination thereof, and can be as few as about 12 nucleotides in length, usually about 14 to 18 nucleotides in length, and possibly as large as the entire sequence of a BEHAB gene. Selection of probe size is somewhat dependent upon the use of the probe, and is well within the skill of the art.

Suitable probes can be constructed and labeled using techniques that are well known in the art. Shorter probes of, for example, 12 bases can be generated synthetically and labeled with ³²P using T₄ polynucleotide kinase. Longer probes of about 75 bases to less than 1.5 kb are preferably generated by, for example, PCR amplification in the presence of labeled precursors such as, but not limited to, [α³²P] dCTP, digoxigenin-dUTP, or biotin-dATP. Probes of more than 1.5 kb are generally most easily amplified by transfecting a cell with a plasmid containing the relevant probe, growing the transfected cell into large quantities, and purifying the relevant sequence from the transfected cells, see Sambrook et al. (1989, Molecular Cloning: A Laboratory Manual, Cold Spring Harbor Laboratory, New York).

Probes can be labeled by a variety of markers, including for example, radioactive markers, fluorescent markers, enzymatic markers, and chromogenic markers. The use of ³²P is but one example for marking or labeling a particular probe.

It is a feature of this aspect of the invention that the probes can be utilized to detect the presence of a BEHAB mRNA or DNA within a sample. However, if the relevant sample is present in only a limited number, then it can be beneficial to amplify the relevant sequence so that it may be more readily detected or obtained.

A variety of methods may be utilized in order to amplify a selected sequence, including, for example, RNA amplification (see Lizardi et al., 1988 Bio/Technology 6:1197-1202; Kramer et al., 1989, Nature 339:401-402; Lomeli et al., 1989, Clinical Chem. 35:1826-1831; U.S. Pat. No. 4,786,600), and DNA amplification utilizing ligase chain reaction (LCR) or PCR (U.S. Pat. Nos. 4,683,195, 4,683,202, and 4,800,159) (see also U.S. Pat. Nos. 4,876,187 and 5,011,769, which describe an alternative detection/amplification system comprising the use of scissile linkages), or other nucleic acid amplification procedures that are well within the level of ordinary skill in the art. With respect to PCR, for example, the method can be modified as known in the art. Transcriptional enhancement of PCR can be accomplished by incorporation of bacteriophage T7 RNA polymerase promoter sequences in one of the primary oligonucleotides, and immunoenzymatic detection of the products from the enhanced emitter may be effected using anti-RNA:DNA antibodies (Blais, 1994, Appl. Environ. Microbiol. 60:348-352). PCR can also be used in combination with reverse dot-blot hybridization (Iida et al., 1993, FEMS Microbiol. Lett. 114:167-172). PCR products may be quantitatively analyzed by incorporation of dUTP (Duplaa et al., 1993, Anal. Biochem. 212:229-236), and samples may be filter sampled for PCR-gene probe detection (Bej et al., 1991, Appl. Environ. Microbiol. 57:3529-3534).

The invention encompasses a method of detecting BEHAB overexpression and therefore diagnosing a primary CNS tumor wherein PCR amplification is used to detect BEHAB DNA. As an example, a DNA sample is denatured at about 92° to about 95° C. in order to generate single-stranded DNA. The DNA sample can be a cDNA generated from RNA. Specific primers are then annealed to the single-stranded DNA at about 37° C. to about 70° C., depending on the proportion of AT/GC in the primers and other factors well known in the art. The primers are extended at about 72° C. with, for example, Taq DNA polymerase or another thermostable DNA polymerase in order to generate the opposite strand to the template. These steps constitute one cycle, which can be repeated in order to amplify the selected sequence. For greater specificity, nested PCR can be performed. In nested PCR, a second amplification is performed using a second set of primers derived from sequences within the first amplified product. The entire coding region of BEHAB may be amplified from, for example, cDNA using an adequate number of primers to generate fragment lengths that are a convenient size for determining their sequence. The number of primers necessary will be well known to one of skill in the art.

The present invention further includes a method wherein, LCR amplification is utilized for amplification. LCR primers can be synthesized such that the 5′ base of the upstream primer is capable of hybridizing to a unique base pair in a desired gene to specifically detect a BEHAB gene.

Within an embodiment of the present invention, the probes can be used in an automated, non-isotopic strategy wherein target nucleic acid sequences are amplified by PCR, and then desired products are determined by, for example, a calorimetric oligonucleotide ligation assay (OLA) (Nickerson et al., 1990, Proc. Natl. Acad. Sci. USA 81:8923-8927).

Primers for the amplification of a selected sequence should be selected from sequences that are highly specific to BEHAB and form stable duplexes with the target sequence. As is well known in the art, the primers should also be non-complementary, especially at the 3′ end, should not form dimers with themselves or other primers, and should not form secondary structures or duplexes with other regions of DNA. In general, primers of about 18 to about 20 nucleotides are preferred, and can be easily synthesized using techniques well known in the art. PCR products, and other nucleic acid amplification products, may be quantitated using techniques well known in the art (Duplaa et al., 1993, Anal. Biochem. 212:229-236; Higuchi et al., 1993, Bio/Technology 11:1026-1030).

The skilled artisan will readily understand, when armed with the present disclosure and the data disclosed herein, that diagnostics can be developed which are capable of detecting the overexpression of BEHAB nucleic acid in a mammal. This is because, as demonstrated by the data elsewhere herein, increased expression of BEHAB in a mammal, when compared to a mammal with normal endogenous BEHAB expression or a mammal with overexpression of a non-cleavable BEHAB mutant, results in larger tumors and decreased survival time. Thereby, determining the level of BEHAB expression in a mammal or cell can be used as a powerful and novel diagnostic technique for the detection of among other things, a primary CNS tumor, and the like.

C. Methods of Identifying Useful Compounds

The present invention further includes a method of identifying a compound that affects expression of BEHAB, including a glycosylation-variant BEHAB isoform, in a cell. The method comprises contacting a cell with a test compound and comparing the level of expression of BEHAB in the cell so contacted with the level of expression of BEHAB in an otherwise identical cell not contacted with the compound. If the level of expression of BEHAB is higher or lower in the cell contacted with the test compound compared to the level of expression of BEHAB in the otherwise identical cell not contacted with the test compound, this is an indication that the test compound affects expression of BEHAB in a cell.

The invention encompasses methods to identify a compound that affects expression of BEHAB. One skilled in the art would appreciate, based upon the disclosure provided herein, that assessing the level of BEHAB can be performed using probes (e.g., antibodies and/or nucleic acid probes that specifically bind with of BEHAB), such that the method can identify a compound that selectively affects expression of BEHAB. Such compounds are useful for inhibiting expression of BEHAB. One skilled in the art would understand that such compounds can be useful for inhibiting a disease, disorder, or condition mediated by and/or associated with increased expression of BEHAB , e.g., increased levels of BEHAB is associated with primary CNS tumors, and BEHAB expression is associated with increased tumor volume and decreased survival rates. Thus, the skilled artisan would appreciate, based on the disclosure provided herein, that it may useful to decrease expression of BEHAB.

Similarly, the present invention includes a method of identifying a compound that reduces expression of BEHAB, including a glycosylation-variant BEHAB in a cell. The method comprises contacting a cell with a test compound and comparing the level of expression of BEHAB in the cell contacted with the compound with the level of expression of BEHAB in an otherwise identical cell, which is not contacted with the compound. If the level of expression of BEHAB is lower in the cell contacted with the compound compared to the level in the cell that was not contacted with the compound, then that is an indication that the test compound reduces expression of BEHAB or a glycosylation-variant BEHAB in a cell.

The invention also includes a method of identifying a compound that decreases cleavage of BEHAB in a cell. The method comprises contacting a cell with a test compound and comparing the level of BEHAB cleavage in the cell contacted with the compound with the level of BEHAB cleavage in an otherwise identical cell, which is not contacted with the compound. If the level of BEHAB cleavage is lower in the cell contacted with the compound compared to the level in the cell that was not contacted with the compound, then that is an indication that the test compound decreases cleavage of BEHAB in a cell.

A compound that decreases BEHAB cleavage in a cell is useful since it has been demonstrated herein that BEHAB cleavage is associated with primary CNS tumor progression and invasiveness. Additionally, the data disclosed herein demonstrate that BEHAB cleavage mediates or is associated with larger tumor volumes and decreased animal survival rates. Thus, methods of identifying a compound that decreases BEHAB cleavage can be used to treat various diseases, including, but not limited to, primary CNS tumors.

The skilled artisan will further appreciate that the present invention is not limited to a method of identifying a useful compound in a cell or an animal. That is, the present invention includes methods of identifying a useful compound in a cell-free system. A cell-free system, as used herein, refers to an in vitro assay wherein the components necessary for a reaction to take place are present, but are not associated with a cell. Such components can include cellular enzymes, transcription factors, proteins, nucleic acids, and the like, provided that they are substantially free from a cell. As disclosed by the data herein, BEHAB cleavage assays can be performed free of a cell or animal, including the use of immunoprecipitation assays and the like. Thereby, the present invention includes a method of identifying a useful compound for treating a primary CNS tumor in a cell-free system.

One skilled in the art would appreciate, based on the disclosure provided herein, that the level of expression of BEHAB in the cell may be measured by determining the level of expression of mRNA encoding BEHAB. Alternatively, the level of expression and/or cleavage of BEHAB can be determined by using immunological methods to assess BEHAB production and cleavage, as exemplified herein using Western blot analysis using anti-BEHAB antibodies. Further, nucleic acid-based detection methods, such as Northern blot and PCR assays and the like, can be used as well. In addition, the level of BEHAB activity and/or cleavage in a cell can also be assessed by determining the level of various parameters which can be affected by BEHAB activity and/or cleavage, such as, for example, tumor volume, tumor invasiveness, and animal survival rates. Thus, one skilled in the art would appreciate, based upon the disclosure and reduction to practice provided herein, that there are a multitude of methods that are well-known in the art which can be used to asses the level of BEHAB activity and cleavage in a cell including those disclosed herein and others which may be developed in the future.

In addition, a protein that specifically binds with BEHAB or its cleavage products, e.g. a receptor or other BEHAB-associated protein, can be identified using, for example, a yeast two hybrid assay. Yeast two hybrid assay methods are well-known in the art and can be performed using well documented techniques, for example those described in Bartel and Fields, (The Yeast Two-Hybrid System, Oxford University Press, Cary, N.C.). Therefore, once armed with the teachings provided herein, e.g., the full amino and nucleic acid sequences of the BEHAB protein, one skilled in the art can easily identify a protein that specifically binds with BEHAB or its cleavage products such as, but not limited to, a BEHAB target or receptor protein.

One skilled in the art would understand, based upon the disclosure provided herein, that the invention encompasses any molecule identified using the methods discussed elsewhere herein. That is, molecules that associate with BEHAB, such as but not limited to, a BEHAB receptor protein, or a BEHAB target protein, can be used to develop therapeutics and diagnostics for diseases, disorders or conditions mediated by BEHAB cleavage product interaction with a BEHAB-associated protein such as primary CNS tumors, gliomas, well-differentiated astrocytomas, anaplastic astrocytomas, glioblastoma multiforme, ependymomas, oligodendrogliomas, ganglioneuromas, mixed gliomas, brain stem gliomas, optic nerve gliomas, meningiomas, pineal tumors, pituitary tumors, pituitary adenomas, primitive neuroectodermal tumors, schwannomas, vascular tumors, and lymphomas. That is, one skilled in the art would appreciate, as more fully set forth elsewhere herein in discussing antibodies that specifically bind with BEHAB, that a BEHAB-associated protein can be used to develop therapeutics that inhibit BEHAB cleavage product activity in a cell by inhibiting BEHAB cleavage product receptor/ligand interactions and other BEHAB binding interactions.

BEHAB-associated proteins identified by the above-disclosed methods can be used directly to inhibit BEHAB interactions by contacting a cell with the BEHAB-associated protein, or a portion thereof, or they can be used to develop antibodies and/or peptidomimetics that can inhibit the BEHAB-associated protein interaction with BEHAB thereby inhibiting BEHAB function, activity, and cleavage. Thus, BEHAB-associated proteins, including a BEHAB receptor proteins or BEHAB cleavage product proteins, are useful and are encompassed by the invention.

VIII. Kits

The present invention encompasses various kits which comprise a compound, including a nucleic acid encoding mutant BEHAB, a mutant BEHAB polypeptide, an antibody that specifically binds BEHAB, a nucleic acid complementary to a nucleic acid encoding BEHAB but in an antisense orientation, an antibody to BEHAB cleavage products, an applicator, and instructional materials which describe use of the compound to perform the methods of the invention. Although model kits are described below, the contents of other useful kits will be apparent to the skilled artisan in light of the present disclosure. Each of these kits is contemplated within the present invention.

In one aspect, the invention includes a kit for treating a primary CNS tumor. The kit is used in the same manner as the methods disclosed herein for the present invention. Briefly, the kit may be used to contact a cell with a nucleic acid encoding a mutant BEHAB molecule of the invention. Additionally, the kit comprises an applicator and an instructional material for the use of the kit. These instructions simply embody the examples provided herein.

The kit further includes a pharmaceutically-acceptable carrier. The composition is provided in an appropriate amount as set forth elsewhere herein. Further, the route of administration and the frequency of administration are as previously set forth elsewhere herein.

In another aspect, the invention includes a kit for treating a primary CNS tumor. The kit is used in the same manner as the methods disclosed herein for the present invention. Briefly, the kit may be used to contact a cell with a mutant BEHAB polypeptide molecule of the invention. Additionally, the kit comprises an applicator and an instructional material for the use of the kit. These instructions simply embody the examples provided herein. The kit further includes a pharmaceutically-acceptable carrier. The composition is provided in an appropriate amount as set forth elsewhere herein. Further, the route of administration and the frequency of administration are as previously set forth elsewhere herein.

The invention further encompasses a kit for the treatment of a primary CNS tumor. The skilled artisan will appreciate that the kit can be used according to the methods set forth herein. The kit comprises an antibody, small molecule, or peptide that binds BEHAB, or some fragment thereof, an applicator, and an instructional material substantially similar to the examples provided herein. The kit further includes a pharmaceutically acceptable carrier, of which the composition, route of administration, and frequency of administration are as previously disclosed elsewhere herein.

Further, the invention comprises a kit comprising an antisense nucleic acid complementary to a nucleic acid encoding a mammalian BEHAB molecule, or some fragment thereof. Such kits can be used according to the methods of the invention to mediate the decreased expression of BEHAB. Additionally, the kit comprises an applicator and an instructional material for the use of the kit. These instructions simply embody the examples provided herein. The kit further includes a pharmaceutically-acceptable carrier. The antisense nucleic acid and pharmaceutically-acceptable carrier are provided in an appropriate amount as set forth elsewhere herein. Further, the route of administration and the frequency of administration are as previously set forth elsewhere herein.

The present invention further encompasses a kit for the treatment of a primary CNS tumor. Briefly, the kit comprises an antibody, a small molecule, or a peptide that specifically binds to BEHAB cleavage products, or some fragment thereof, and can be used according to the methods set forth elsewhere herein. The kit of the invention further comprises an applicator and an instructional material, similar to the methods set forth herein, for the use of the kit. The kit also comprises a pharmaceutically-acceptable carrier, of which the composition, route and frequency of administration, and dosage are set forth previously herein.

The present invention further comprises a kit for detecting a glycosylation-variant BEHAB isoform. The kit comprises an antibody to a glycosylation-variant BEHAB isoform. Such antibodies are disclosed are set forth elsewhere herein. The kit further comprises an instructional material comprising information on how to use the antibody for the detection of a glycosylation-variant BEHAB isoform, including instructions to accomplish the methods set forth elsewhere herein.

The present invention further comprises a kit for diagnosing a primary CNS tumor in a mammal. The kit comprises an antibody that specifically binds a glycosylation-variant BEHAB isoform, an applicator and a instructional method for the use of the kit. Uses of an applicator and methods for the diagnosis of a primary CNS tumor are disclosed elsewhere herein.

The invention also includes a kit for treating a primary CNS tumor. The kit includes a composition comprising an antibody that specifically binds a glycosylation-variant BEHAB isoform, or a fragment thereof, a pharmaceutically acceptable carrier, and an applicator. Methods for using an antibody and applicator are set forth elsewhere herein. The instructional material comprises the methods disclosed herein for the treatment of a primary CNS tumor.

The invention further includes a kit for treating a primary CNS tumor using immune therapy. The kit comprises a glycosylation-variant BEHAB isoform, or a fragment thereof, for use in stimulating a cell derived from a mammal to treat a primary CNS tumor. The kit further comprises an applicator for administration of the glycosylation-variant BEHAB-stimulated cell to a mammal after the cell has been stimulated to provide immune therapy to a mammal afflicted with a primary CNS tumor. The kit also includes an instructional material comprising the methods disclosed herein for the immune therapy of a primary CNS tumor.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

The materials and methods used in the experiments presented in this Example are now described.

Site-Directed Mutagenesis: Full-length BEHAB cDNA was cloned into the EcoR1 site of the eukaryotic expression vector pcDNA3 (Invitrogen, Carlsbad, Calif.) as described in Zhang et al. (1998, Journal of Neuroscience 18: 2370-2376). The full-length BEHAB expression vector was mutated using the QUIKCHANGE site-directed mutagenesis kit following the manufacturer's protocol (Stratagene, La Jolla, Calif.). Incorporation of the appropriate mutation was confirmed by sequencing using the fluorescently-labeled dideoxynucleotide chain termination method.

Cell Culture and Transfections: CNS-1 cells (American Type Culture Collection, Manassas, Va.) were grown and maintained in RPMI with 10% fetal calf serum (FCS). Cells were split (1:6 to 1:10) and re-plated every four days. 75% confluent cells were plated in 60 mm tissue culture plates and transfected using 4 μg of the appropriate expression construct and Fugene 6 according to the manufacturer's protocol (Roche, Indianapolis, Ind.). Briefly, DNA and Fugene 6 were incubated with cells in their standard media for six hours, after which the media was removed and replaced with fresh media overnight. The following day, cells were selected for expression of the transgene with G-418 (80 μg/μl, Invitrogen). Stable pools of transfected cells were derived by maintaining cells in G-418-supplemented media for two and a half weeks. After selection, stably transfected pools of cells were maintained in media containing G-418 at a concentration of (40 μg/μl).

In vitro proliferation and cell death assays: The effects of transfections on cell proliferation and cell death were evaluated. For both assays, cells were grown in 96 well tissue culture plates with an initial plating density of 4×10⁴ cells per well in 200 μl of media. Cell proliferation was measured by cellular uptake of MTT (3,-[4,5-dimethylthiazol-2-yl]-2,5-diphenyltetrazolium bromide; Sigma Chemical Co., St. Louis, Mo.). Briefly, 100 μl of media was removed was removed and saved for the cell death assays. 25 μl of MTT (2 mg/ml in Dulbecco's phosphate buffered saline; DPBS) was added to the remaining cells and media and incubated for 3 hours at 37° C. in a humidified incubator (5% CO₂ in air). 100 μl of 40 mM hydrochloric acid in isopropanol was added to solubilize the resultant product and samples were incubated at 37° C. for 2 hours. Samples were analyzed in quadruplicate by measuring the absorbance at 550 nM using a microplate reader.

100 μl of cell-free media was used for the cell death assays. The media was assayed for lactate dehydrogenase (LDH) activity using the Cytotoxicity Detection Kit per the manufacturer's instructions (Roche, Indianapolis, Ind.). Briefly, media was incubated with lactate, which is oxidized to pyruvate by LDH and reduces NAD⁺ to NADH. NADH in the presence of diaphorase catalyzes the conversion of yellow tetrazolium salt into red formazan salt. Samples were analyzed in quadruplicate by measuring the absorbance at 450 nM using a microplate reader.

Animal Studies: Female Lewis rats (Charles River Laboratories, Wilmington, Mass.) were anesthetized (75 mg/kg ketamine, 5 mg/kg xylazine) and positioned in a stereotaxic instrument (David Kopf Instruments, Tujunga, Calif.) with the incisor bar set at 3.0 mm below the intraaural line. About 70%-80% confluent CNS-1 cells were harvested from 100 mm tissue culture plates using trypsin. Cells were washed once in DPBS and suspended in phosphate buffered saline (PBS) supplemented with 1 μg/μl MgCl₂, 1 μg/μl CaCl₂, and 0.1% glucose at a concentration of 5×10⁴ cells/μl, except for the survival curve with the inhibitor in which the concentration of cells was 1×10⁵ cells/μl. Intrathalamic injections were made using a 10 μl Hamilton syringe fitted with a 26 gauge beveled needle into the right thalamus at the coordinates 2.8 mm posterior to bregma, 2.2 mm lateral to the midline, and 5.0 mm ventral to the dura. A total volume of 3 μl of the cell suspension was injected over 3 minutes and the needle was left in place for an additional minute before slow withdrawal.

The health and survival rate of the animals was evaluated every six hours. Animals that were not able to right themselves within 15 seconds of being placed on their side were considered to have reached the survival endpoint and sacrificed. The day of sacrifice was recorded as the last day of survival.

Histology: Rats were deeply anesthetized and transcardially perfused with PBS followed by ice-cold 4% PBS-paraformaldehyde. 40 μM frozen sections were sliced on a cryostat and brains from animals implanted with transfected CNS-1 cells were sliced coronally. Every fifth section was stained with cresyl violet for estimation of tumor volumes.

Image Analysis and Tumor Volume Estimation: Tissue sections were analyzed using NIH Image (http://rsb.info.nih.gov/nih-image/). Tumor areas were defined manually on every fifth section through the tumor by a researcher blinded to the experimental conditions. Tumor volumes were reconstructed using Calvalieri's estimator of morphometric volume (Rosen and Harry, 1990, Journal of Neuroscience Methods 35: 115-124).

Electrophoresis and Western Analysis: Samples were electrophoresed on 8% SDS-PAGE gels and proteins were then electrophoretically transferred to nitrocellulose (Laemmli et al., 1970, Nature 227:680-685; Towbin et al., 1979, Proc. Natl. Acad. Sci. U.S.A. 76:4350-4354). Blots were incubated with rabbit affinity purified antisera (1:10,000) or B50 (1:5000) (Matthews et al., 2000, J. Biol. Chem. 275: 22695-22703) followed by alkaline phosphatase-conjugated goat anti-rabbit IgG secondary antibody (1:4000, Jackson ImmunoResearch Labs, West Grove, Pa.). Immunoreactive bands were visualized with nitro blue tetrazolium and 5-bromo-4-chloro-3-indoyl phosphate.

Statistical Analysis: The significance of comparisons between animals implanted with transfected cell lines was determined by one-way analysis of variance (ANOVA). Results from the in vitro proliferation and cell death assays were analyzed using a 4×7 (cell line×day) repeated measures ANOVA.

The results of the experiments presented in this Example are now described.

The Effect of the NVY Mutation on BEHAB Cleavage: To study the role of BEHAB cleavage in primary CNS tumors, a construct was generated that blocked or inhibited BEHAB cleavage. The effect of mutating the BEHAB Glu³⁹⁵-Ser³⁹⁶ cleavage site on cleavage of the full length protein was examined. Previous work on aggrecan, which has a cleavage site very similar to the BEHAB Glu³⁹⁵-Ser³⁹⁶ site, showed that mutation of the three amino acids just downstream of the cleavage site from ARG to NVY completely blocked cleavage (Fosang et al., 2000, J. Biol. Chem. 275: 33027-33037). Therefore, using site-directed mutagenesis, the effect of the NVY mutation on BEHAB cleavage was examined. This mutation changed the amino acid cleavage site from ³⁹³Glu-Ser-Glu-Ser-Arg-Gly³⁹⁸ to ³⁹³Glu-Ser-Glu-Asn-Val-Tyr³⁹⁸ (SEQ ID NO:1 and SEQ ID NO:2, respectively). To evaluate the effect of the NVY mutation on BEHAB cleavage, CNS-1 cells were transiently transfected with either full-length BEHAB, or with the mutated BEHAB construct and the resultant protein was analyzed by Western blot with the B6 and B50 antibodies (specifically binding the C-terminus and the neo-epitope formed by BEHAB cleavage, respectively, Matthews et al., 2000. J. Biol. Chem. 275: 22695-22703).

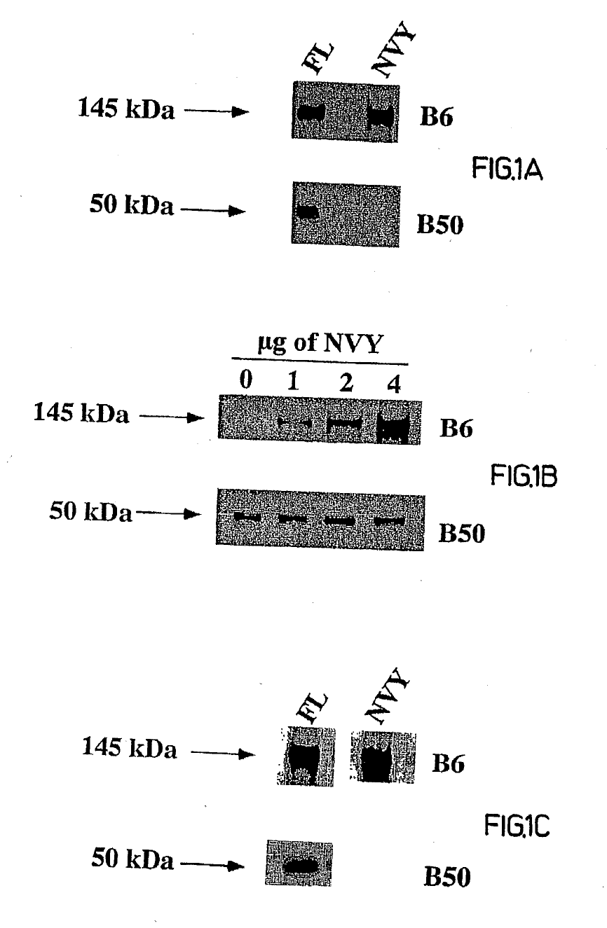

Cells transiently transfected with both the normal and mutated BEHAB produced the full-length product as shown with the B6 antibody (FIG. 1A). Normal full-length BEHAB was cleaved at the ³⁹³Glu-Ser-Glu-Ser-Arg-Gly³⁹⁸ cleavage site as shown with the B50 antibody. In sharp contrast, while a large amount of full-length protein was made by cells transfected with the NVY mutant construct, no cleavage was detected (FIG. 1A). To determine if the NVY mutated form of BEHAB was completely incapable of being cleaved, samples were immunoprecipitated with the B50 antibody to detect trace amount so cleavage product. Even with this higher sensitivity assay, no reactive product was detected, indicating that the NVY mutation completely inhibited cleavage of the full-length protein.

The effect of the NVY mutation on cleavage of BEHAB was further investigated by co-transfecting CNS-1 cells with the normal full-length BEHAB and NVY constructs. These experiments were designed to determine if the NVY mutant BEHAB could inhibit cleavage of the normal substrate. Cells were co-transfected with 2 μg of the full-length BEHAB expression construct and either 0, 1, 2, or 4 μg of the mutated BEHAB construct. While the addition of the NVY mutant construct increased the amount of full-length BEHAB, it had no effect on the ability of the protease to cleave the full-length BEHAB protein (FIG. 1B). These results suggest that, although not wishing to be bound by any particular theory, while the mutated form of BEHAB is itself uncleavable, it does not necessarily inhibit the protease.

The Effect of the NVY Mutant on Cell Proliferation and Cell Death:

To evaluate the effects of the NVY cleavage site mutation of BEHAB on cell proliferation and cell death, CNS-1 cells were stably transfected with either a green fluorescent protein (GFP) expression vector (CNS-1-GFP), the normal BEHAB expression vector (CNS-1-FL), or the NVY mutated vector (CNS-1-NVY). Pools of stably transfected cells were selected in G418 and analyzed as described above. As observed using transient transfections, pools of CNS-1-NVY cells made full-length BEHAB, but did not cleave it (FIG. 1C).

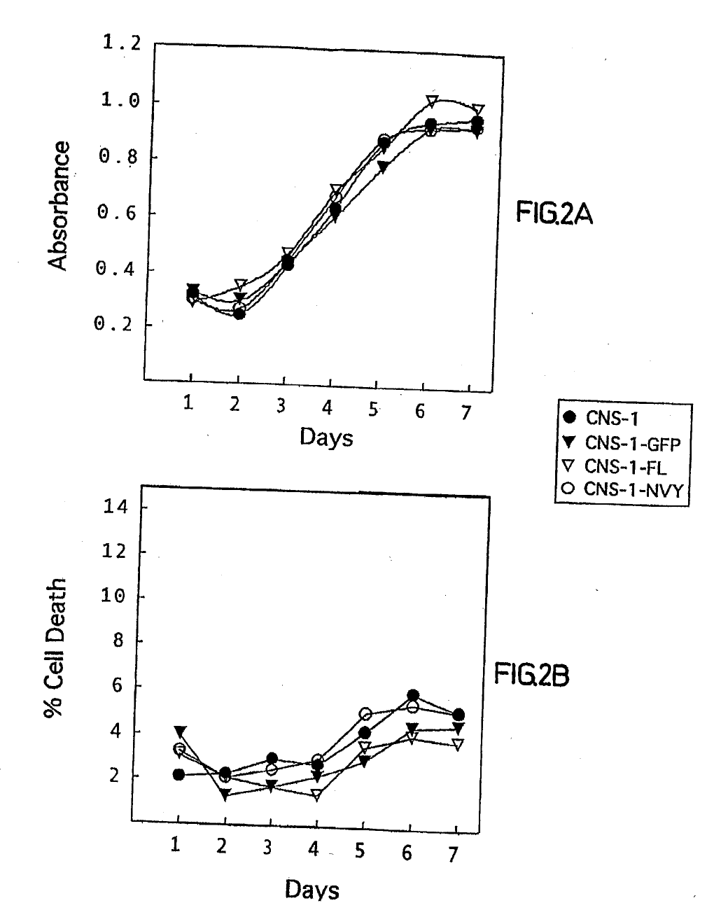

Cell proliferation was analyzed in vitro on stably transfected cells using an MTT assay. MTT is converted to a dye by the succinate-tetrazolium reductase system, an enzyme system of the mitochondrial respiratory chain, thereby providing a measure of the number of viable cells. Proliferation was evaluated over seven days. There were no differences in cell proliferation in any of the transfected cells (FIG. 2A). Furthermore, none of the transfected pools showed any differences from the non-transfected parental CNS-1 cells.

Cell death was evaluated using the LDH assay. LDH is an enzyme present normally only in the cytoplasm. LDH activity was measured in the media, where LDH would only be present if released by dead or dying cells. There was a low level of about 2% to about 3% cell death over the first four days in all cell lines. Cell death increased slightly on every subsequent day, however, there were no differences in the percent dell death between any cell line at any time point (FIG. 2B). Therefore, while the mutated construct is uncleavable, it has no apparent effect on cell proliferation or death in vitro.

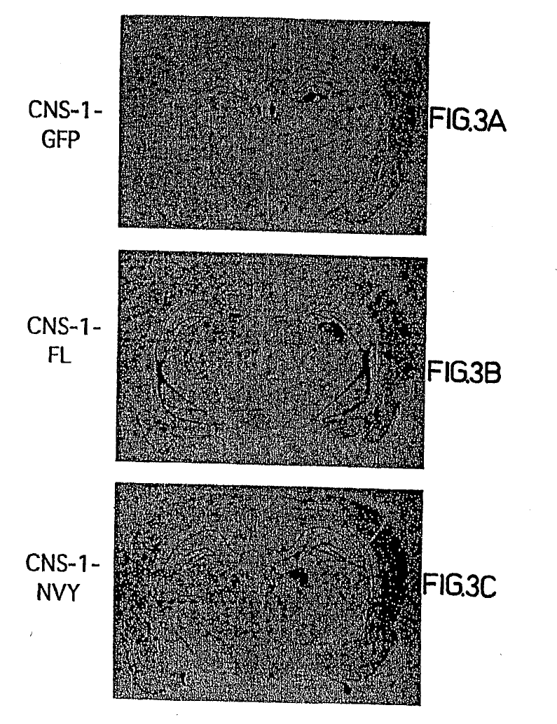

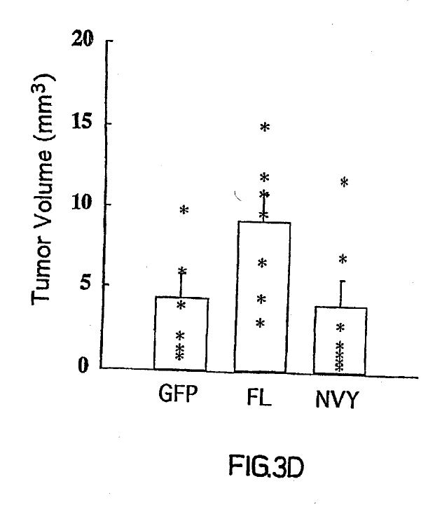

The NVY Uncleavable BEHAB Does Not Affect the Phenotype of CNS-1 Tumors: Previous studies have indicated that CNS-1 cells transfected with the normal full-length BEHAB form larger tumors than GFP transfected controls. However, in 9L cells, which neither produce nor cleave BEHAB, transfection with full-length BEHAB does not change the phenotype of the tumors (Zhang et al., 1998, J. of Neuroscience 18: 2370-2376). To investigate the role of BEHAB cleavage in tumor progression, pools of CNS-1 cells stably transfected with the expression vectors described above were grown as intracranial grafts. Eight days after tumor implantation, animals (n=8) were perfused and their brains processed for histology. Every fifth section was stained with cresyl violet and tumor areas were estimated by image analysis. Volumes were reconstructed from these analyses. Consistent with previous results, CNS-1-FL tumors were larger and more infiltrative than CNS-GFP. CNS-1-NVY tumors were not significantly different in size from CNS-1-GFP tumors. Importantly however, CNS-1-NVY tumors were significantly smaller than CNS-1-FL tumors (Table 1, FIG. 3). While not wishing to bound by any particular theory, these results demonstrate that production of BEHAB alone is insufficient to increase tumor progression, but cleavage of the full-length protein plays a critical role in this process.

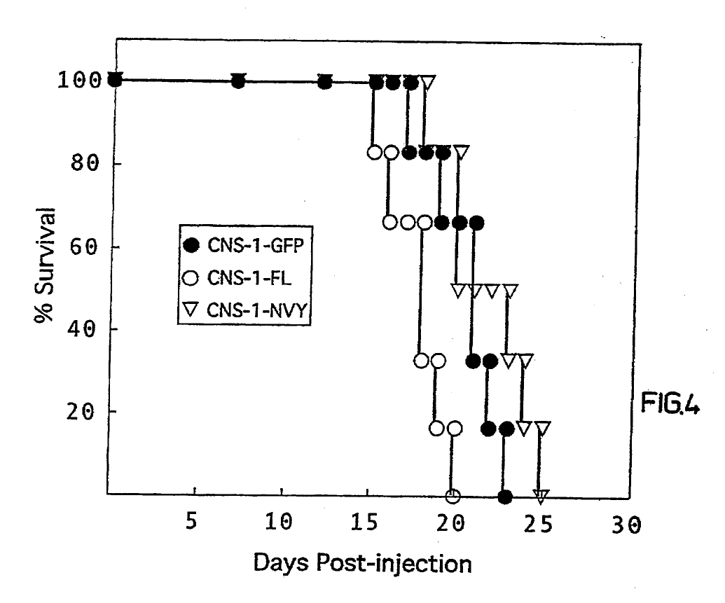

To further investigate the effects of mutated BEHAB on tumor progression, the effect of the mutant BEHAB construct on animal (n=6) survival was investigated. It was previously discovered that CNS-1-FL tumors decrease animal survival (Nutt et al., 2001, Cancer Res. 61: 7056-7059). Consistent with these results, animals implanted with CNS-1-FL tumors reached the survival endpoint an average of three days earlier than animals implanted with CNS-1-GFP tumors. However, CNS-1-NVY implanted animals did not reach the survival endpoint faster than controls. In fact, animals with CNS-1-NVY tumors survived slightly longer than animals implanted with CNS-1-GFP, although with the number of animals in this study to date, the difference is not significant. CNS-1-NVY implanted animals survived an average of four days longer than CNS-1-FL implanted animals, a 23% increase in survival (Table 1, FIG. 4).

TABLE 1

| | Tumor | Significance | | Significance |
|---|---|---|---|---|
| | Volume | Compared to | Survival in | Compared to |
| Tumor Type | in mm³ | CNS-1-FL | Days | CNS-1-FL |
| CNS-1-FL | 9.2 ± 4.3 | | 17.6 ± 0.76 | |
| CNS-1-GFP | 4.4 ± 3.4 | p < 0.05 | 20.5 ± 0.88 | p < 0.05 |
| CNS-1-NVY | 4.0 ± 4.2 | p < 0.05 | 21.6 ± 1.1 | p < 0.05 |

### EXAMPLE 2

### Subcellular Fractions Of Rat Brain

Female Lewis rats of several postnatal ages were deeply anesthetized under halothane and sacrificed by decapitation. Embryos of 14 to 18 gestational days were quickly removed from terminally anesthetized pregnant rats onto ice and decapitated. Forebrains were quickly dissected on ice and homogenized in 10 volumes of 25 mM TrisHCl, pH 7.4, containing 0.32 M sucrose (TS buffer) and protease inhibitor cocktail (Complete EDTA-free, Roche, Indianapolis, Ind.). The homogenate was centrifuged at 950 g for 10 minutes and the nuclear pellet (P1) was washed once by rapid rehomogenization in TS buffer and centrifuged as before. The post-nuclear supernatants were combined and centrifuged at 100,000 g for 60 minutes to prepare total particulate and soluble fractions.

For further subcellular fractionation from adult rat forebrain samples, the postnuclear supernatant, obtained as indicated above, was centrifuged according to established protocols (Rodriguez de Lores et al, 1967, J. Neurochem 14:215-225),yielding a mitochondrial/synaptosomal pellet (P2), a light microsomal pellet (P3) and a final soluble fraction. To separate mitochondrial and synaptosomal membranes, the P2 pellet was resolved in a discontinuous sucrose gradient as described in, for example (Jones and Matus, 1974, Biochim. Biophys. Acta 356: 276-287). To prepare subcellular fractions for protein electrophoresis, aliquots of membrane and soluble fractions were equilibrated at a final total protein concentration of 1-2 mg/ml in 40 mM TrisHCl/40 mM sodium acetate, pH 8 (CH buffer), containing 10 mM EDTA, and treated with 0.25 U/ml of protease-free chondroitinase ABC from Proteus vulgaris (EC 4.2.2.4, Seikagaku, Falmouth, Mass.) for 8 hours at 37° C. Chondroitinase reaction was stopped by boiling the samples in the presence of 1×gel-loading buffer.

### Release Of BEHAB Isoforms From Brain Membranes

To characterize the association of different BEHAB isoforms with the cell membrane, total membranes (1 to 2 mg total protein/ml) obtained from rat forebrain were resuspended in 50 mM TrisHCl buffer, pH 7.4, in the presence or absence of 10 mM EDTA or 0.2% Triton X-100, for 1 hour at 4° C. Alternatively, membranes were resuspended in 100 mM sodium carbonate, pH 11.3, for 30 minutes at 4° C. After incubation, membranes were centrifuged at 20,800 g for 20 minutes. Released BEHAB was recovered in the supernatant, and the membranes containing retained BEHAB were washed twice with 50 mM TrisHCl buffer and resuspended in the same initial volume. All samples were finally equilibrated with CH buffer and treated with chondroitinase ABC prior to protein electrophoresis. For immunoprecipitation studies, membranes were extracted for 1 hour at 4° C. in 50 mM TrisHCl, pH 7.4, containing 0.6% w/v CHAPS, and further processed according to standard protocols.

### PI-PLC Treatment And Detergent Extraction With Triton X-114

To reveal the GPI anchored isoform of BEHAB, rat brain microsomal membranes were resuspended in CH buffer with 0.25 U/ml chondroitinase ABC and 1 U/ml of phosphatidylinositol-specific phopholipase-C from Bacillus cereus (PI-PLC, EC 3.1.4.10, Sigma, St. Louis, Mo.) for 8 hours at 37° C. Samples were subsequently separated by SDS-PAGE. To determine the electrophoretic mobility of GPI anchored BEHAB before releasing its GPI anchor, a protein extraction was performed on chondroitinased rat brain membranes using the detergent Triton X-114 (Sigma, St. Louis, Mo.). Triton X-114 allows the separation of water-soluble membrane-associated proteins from hydrophobic or GPI-bound proteins, according to a previously established procedure (Bordier, 1981, J. Biol. Chem. 256:1604-1607). Briefly, microsomal membranes were resuspended in CH buffer at 2 mg total protein/ml and detergent extracted for 60 minutes on ice by adding precondensed 10% v/v Triton X-114 to a final concentration of 2% v/v. Solubilized proteins were recovered in the supernatant after centrifuging the extract at 20,800 g for 20 minutes in a refrigerated centrifuge. This supernatant was warmed at 37° C., producing an aqueous phase and a detergent-containing phase. Both phases were isolated by centrifugation at 2000 g for 10 minutes at room temperature and exhaustively washed to avoid cross-contamination. Extracted proteins in the detergent phase were precipitated with acidic ethanol at −20° C., washed with cold acetone and re-solubilized in a small volume of CH buffer in the presence of 0.6% w/v CHAPS. Samples from the aqueous and detergent phases were treated with PI-PLC for 8 hours at 37° C. before SDS-PAGE.

### Cell Cultures And Transfections

The rat CNS-1 glioma cell line (generously provided by Dr. W. Hickey, Darthmouth-Hitchcock Medical Center, Lebanon, N.H.) was grown at 5% CO₂ in RPMI-1640 medium supplemented with 10% FCS (Hyclone, Logan Utah), 50 μg/ml penicillin and 50 μg/ml streptomycin (Gibco, La Jolla, Calif.) (Kruse et al, 1994, J. Neurooncol. 22: 191-200). The mouse oligodendrocyte precursor Oli-neu line (generously provided by Dr. J. Trotter, Department of Neurobiology, University of Heidelberg, Germany) was grown on poly-D-Lysine precoated plates (Beckton-Dickinson, Franklin Lakes, N.J.) in Sato's medium (Jung et al, 1995, Eur. J. Neurosci. 7: 1245-1265) supplemented with 25 μg/ml Geneticin (Gibco, La Jolla, Calif.). CNS-1 cells were transfected as described in, for example Zhang et al (1998, J. Neurosci. 18: 2370:2376) with a pcDNA3.1 vector (Invitrogen, La Jolla, Calif.) containing the full-length rat BEHAB cDNA (nucleotides 1-2863, generously provided by Dr. Yu Yamaguchi, Burnham Institute) (Yamada et al, 1995, Biochem. Biophys Res. Commun. 216: 957-963). Control cells were transfected with a pcDNA3.1 vector containing a cDNA insert encoding GFP (generously provided by Dr. Tom Hughes, Yale University). Stable transfectants were selected in 1 mg/ml Geneticin (Gibco, La Jolla, Calif.). Oli-neu cells were transiently transfected with the same rat BEHAB cDNA. In addition, the full-length rat BEHAB cDNA was subcloned in a pcDNA3.1IV5-6xHis vector, to produce V5/6xHis-tagged BEHAB, which was also transiently transfected in Oli-neu cells. In all cases, the expression of the desired transgene was confirmed by Northern blot analysis (Zhang et al, 1998, J. Neurosci. 18: 2370:2376) and the presence of BEHAB protein was confirmed by Western blot analysis, as described elsewhere herein.

### Preparation Of Cell Membranes And Immunocytochemistry

Cells were routinely collected 24-48 hours post-transfection and homogenized in 25 mM phosphate buffer, pH 7.4, containing a protease inhibitor cocktail (Complete, EDTA-free, Roche, Indianapolis, Ind.) and 2 U/ml RNAse-free DNAse I (Roche, Indianapolis, Ind.). Total membranes were obtained by centrifugation at 20,800 g×30 min and prepared for protein electrophoresis.

For live immunocytochemical staining of transfected Oli-neu cells, cultures were grown on glass coverslips in 24-well plates for 24-48 hours before transfection with the V5/6xHis-tagged full-length BEHAB cDNA. Unfixed, unpermeabilized cultures were rinsed in DMEM (Gibco, La Jolla, Calif.) without serum and then incubated with a monoclonal anti-V5 antibody (Invitrogen, La Jolla, Calif.) at 4° C. for 30 min. Cultures were rinsed in DMEM, fixed for 20 minutes in 4% paraformaldehyde, pH 7.4, rinsed and then incubated for 60 minutes with Alexa-conjugated goat anti-mouse IgG₁ secondary antibody (Molecular Probes, Eugene, Oreg.). Cultures were finally rinsed in PBS, briefly counterstained with propidium iodide (0.2 μg/ml) and prepared for fluorescence microscopy.

### Intracranial Grafts

Intracranial grafts of stably transfected CNS-1 cells were performed as described in, for example, Jaworski et al (1996, Cancer Res. 56:2293-2298). Briefly, cells were harvested at 80% confluence, washed in PBS and resuspended in injection buffer (PBS supplemented with 1 μg/ml MgCl₂, 1 μg/ml CaCl₂ and 0.1% w/v D(+)glucose) at a concentration of 5×10⁴ cells/μl. The cell suspension (3 μl) was injected stereotaxically into the thalamus of 45-day-old female Lewis rats over a 5-minute period. The animals were returned to their cages and monitored for signs of compromised neurological functions during a two-week period. After two weeks, rats were terminally anesthetized and decapitated, and brains were quickly dissected, frozen on dry ice and stored at −70° C. for further processing. Brains were grossly sectioned and samples were obtained from the visualized tumors as well as from the equivalent regions of the contralateral side of the brain, where tumors were not detected. Soluble and particulate fractions of normal rat brain and rat brain gliomas were prepared as described elsewhere herein.

### Glycosidase Treatments

To remove O-linked oligosaccharides present in BEHAB isoforms, chondroitinased samples were equilibrated in 10 mM TrisHCl, 10 mM sodium acetate, 100 mM NaCl, pH 7, and treated with 20 mU/ml O-glycosidase from Diplococcus pneumoniae (EC 3.2.1.97, Roche, Indianapolis, Ind.) and 100 mU/ml neuraminidase from Arthrobacter ureafaciens (EC 3.2.1.18, Roche, Indianapolis, Ind.). Similarly, N-linked oligosaccharides were removed by incubation with 100 U/ml glycopeptidase F from Chryseobacterium meningosepticum (EC 3.5.1.52, Sigma, St. Louis, Mo.). In all cases, samples were incubated with the enzymes for 8 hours at 37° C. in the presence of protease inhibitors. Enzyme digestions were stopped by boiling the samples in 1×gel-loading buffer.

### Western Blot Analysis

Samples were electrophoresed on reducing 7% SDS polyacrylamide gels and proteins were electrophoretically transferred to nitrocellulose. Blots were incubated with an affinity-purified rabbit polyclonal antibody (B6) produced against a synthetic peptide corresponding to the GAG attachment region (amino acids 506-529) of rat BEHAB. Alternatively, BEHAB was detected by using affinity-purified rabbit polyclonal antibodies produced against synthetic peptides corresponding to the amino acids 60-73 of rat BEHAB (antibody B5) and the amino acids 859-879 of human BEHAB (antibody B_{CRP}). The 50-kDa cleavage product of BEHAB was detected with an antibody (B50) directed against the neoepitope originated by the proteolytic processing of the full-length protein. The antibodies B6, B5 and B50 have been previously described for the detection or rat BEHAB in rat brain samples (Matthews et al, 2000, J. Biol. Chem. 275: 38885-38890). V5/His-tagged recombinant full-length BEHAB was detected with a monoclonal antibody anti-V5 epitope (Invitrogen, La Jolla, Calif.). In all cases, alkaline-phosphatase conjugated secondary antibodies were used. Immunoreactive bands were visualized with nitro-blue tetrazolium and 5-bromo-4-chloro-3-indoyl phosphate.

The results of the experiments presented in this Example are now described.

### Identification Of A Novel, Developmentally Regulated, BEHAB Isoform In The Rat Brain

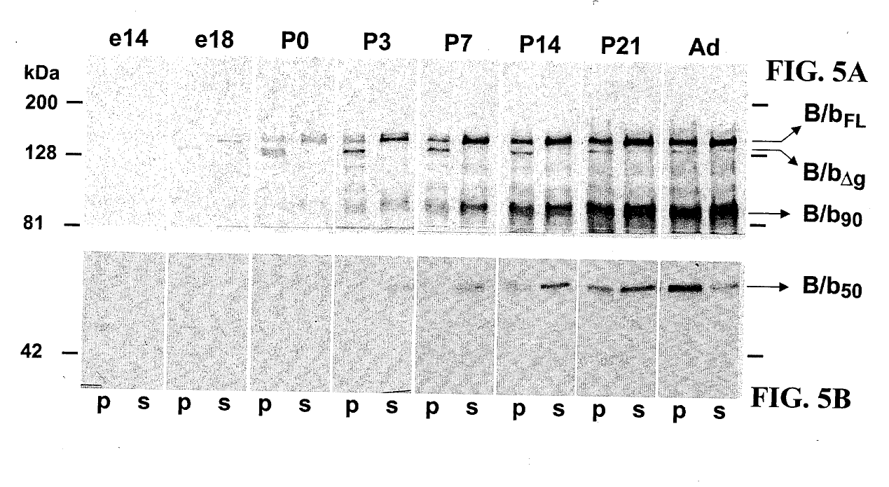

BEHAB protein was detected in the rat brain as early as embryonic day 18 (E18), with levels increasing during postnatal development to reach the adult level of expression by postnatal day 21 (P21) (FIG. 5). The lack of detectable protein at E14 was consistent with previous observations demonstrating BEHAB mRNA was first detected by in situ hybridization in the rat cortex only after E16 (Jaworski et al, 1995, J. Neurosci. 15: 1352-1362). After removal of GAG chains by chondroitinase treatment, the largest isoform of BEHAB migrated at about 150 kDa (full-length BEHAB). The full-length BEHAB isoform was detected predominantly in the soluble fraction early in development but later was found in both soluble and particulate fractions.

In parallel with the increase in expression of full-length BEHAB over the course of development, the major cleavage products of full-length BEHAB also increased (FIG. 5). Full-length BEHAB and its cleavage products are first detected in the soluble fraction, but over the course of development localize increasingly to the particulate fraction of rat brain homogenates.

The immunochemical analysis disclosed herein also reveal a new, glycosylation-variant BEHAB isoform with a molecular weight of about 130-kDa in the particulate fraction. The glycosylation-variant BEHAB was upregulated during early postnatal development, from P3 to P14, while peak expression of full-length BEHAB was later in development, from P21 to adulthood.

### Glycosylation-Variant BEHAB Is Distinct From the GPI-linked BEHAB Isoform

The glycosylation-variant BEHAB isoform and the GPI-linked BEHAB isoform co-localize in the particulate fraction of rat brain isolates. Given the common particulate localization of the glycosylation-variant BEHAB isoform and GPI-linked isoform, the differences between these two isoforms was investigated. Both full-length BEHAB and the glycosylation-variant BEHAB were released from the particulate fraction by non-ionic detergents such as Triton X-100 (FIG. 6A) and zwitterionic detergents such as CHAPS. Indeed, both isoforms were released by detergent concentrations as low as 0.2% w/v Triton X-100, indicating a particular sensitivity to disruption of their hydrophobic interactions with the membranes. This observation suggested that glycosylation-variant BEHAB may not be a GPI-linked protein, since many GPI-linked proteins sit in plasma membrane rafts and tend to have low solubility in Triton X-100 (Schroeder et al, 1994, Proc. Natl Acad. Sci. USA 91: 12130-12134). However, the GPI-linked BEHAB isoform was sensitive to Triton X-100 extraction (Seidenbecher et al, 1995, J. Biol. Chem. 270: 27206-27212). Conversely, brief treatment with alkaline sodium carbonate, which releases membrane associated but not GPI-linked or integral membrane proteins, also released both full-length BEHAB and glycosylation-variant BEHAB isoform from the particulate fraction (FIG. 6A). Together, these results indicate that both full-length BEHAB and glycosylation-variant BEHAB are likely peripherally associated with the cell membrane.

To further test whether glycosylation-variant BEHAB might be the GPI-linked BEHAB isoform, membranes from adult rat brain were treated with PI-PLC, an enzyme that can release GPI-linked proteins. PI-PLC treatment generated a band of approximately 120 kDa, which was clearly distinguished from full-length BEHAB and glycosylation-variant BEHAB in straight 7% acrylamide gels (FIG. 6B). This 120-kDa band was entirely released by PI-PLC, and was not detected in the resulting particulate (membrane) fraction (FIG. 6C). Treatment of the soluble fraction of the brain homogenate with PI-PLC did not give rise to a 120-kDa band, indicating that the GPI-linked isoform resulting in the 120-kDa band specifically localized to the particulate fraction, as would be predicted for a GPI-linked protein. The resulting supernatant (s) and pellet fractions (p) were analyzed by western blot, showing that the 120-kDa band is completely released from membranes by PI-PLC treatment, while the 130-kDa band is not released. A BEHAB isoform in the aqueous phase does not shift in mobility after PI-PLC treatment (H₂O+PLC) but the 150-kDa band in the detergent phase shifts to approximately 120 kDa after PI-PLC treatment (Tx+PLC), confirming that this is the GPI-linked splice variant of BEHAB (FIG. 6). Arrows indicate full-length BEHAB (B/b_{FL}), glycosylation-variant BEHAB (B/b_{Δg}) and GPI-linked BEHAB isoforms.

Full-length BEHAB has been detected with and without GAG addition (Yamaguchi, 1996, Perspect. Dev. Neurobiol. 3: 307-317). Therefore, because detection of the 120-kDa band released by PI-PLC required prior treatment with chondroitinase ABC, the data suggested that the majority of the GPI-linked isoform is invested with chondroitin sulfate proteoglycans.

While PI-PLC treatment led to the evolution of the 120-kDa band, the immunoreactivity of glycosylation-variant BEHAB was unaffected. This result indicated that the 120-kDa band was not derived from glycosylation-variant BEHAB and thus that the glycosylation-variant BEHAB was not the GPI-linked BEHAB isoform. Because there was no obvious depletion of any of the immunoreactive bands following PI-PLC treatment of the GPI-anchored BEHAB isoform, the molecular weight the GPI-anchored BEHAB isoform prior to removal of its lipid anchor was investigated. In order to determine the molecular weight of the GPI-anchored BEHAB isoform, the GPI-linked isoform was isolated from all other BEHAB isoforms prior to the removal of the lipid anchor. To that end, the membrane fraction was extracted with the detergent Triton X-114.

Triton X-114 forms a homogeneous solution with water at 4° C. but partitions into an aqueous phase and a detergent phase at 37° C. Only proteins with significant hydrophobic domains, including integral membrane and GPI-linked proteins, partition into the detergent phase, while membrane associated proteins partition into the aqueous phase (Bordier, 1981 J. Biol. Chem. 256: 1604-1607). Accordingly, all BEHAB isoforms should partition to the aqueous phase, leaving only the GPI-anchored isoform in the detergent phase. Triton X-114 extraction resulted in only a single band of approximately 150-kDa band in the final detergent phase (FIG. 6D). When proteins precipitated from this phase were treated with PI-PLC, the 150-kDa band disappeared and was replaced by a band of approximately 120-kDa, the position previously observed for the GPI-linked BEHAB isoform without its anchor. Further, PI-PLC treatment of the proteins in the watersoluble phase, which included glycosylation-variant BEHAB, did not lead to any loss of the 150-kDa band and did not lead to the appearance of a band of approximately 120-kDa. These results confirmed that glycosylation-variant BEHAB is not the glypiated isoform of BEHAB. Further, the results indicate that prior to removal of its lipid anchor, GPI-linked BEHAB runs at a similar molecular weight as the predominant secreted isoform, full-length BEHAB, thus simultaneous detection of the two forms is difficult without prior extraction.

### Glycosylation-variant BEHAB Is Not A Cleavage Product Of Full-length BEHAB

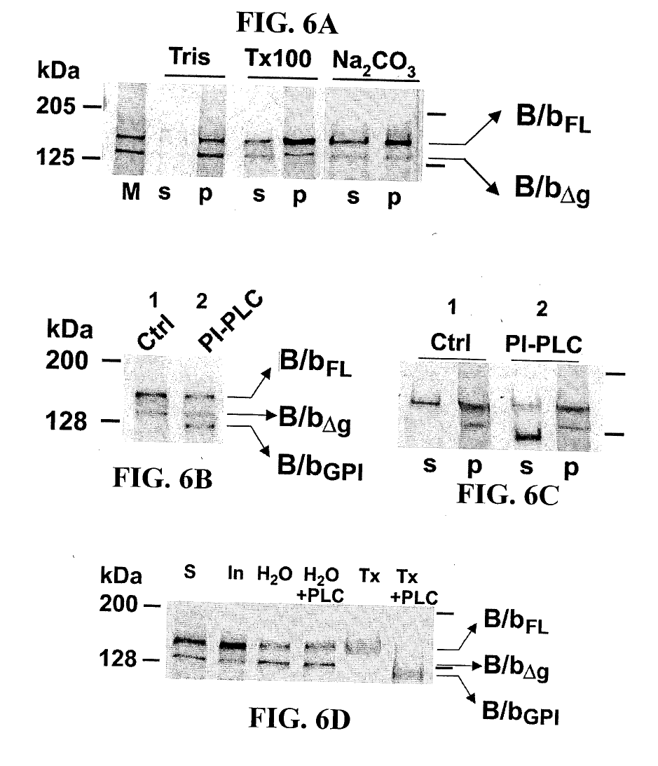

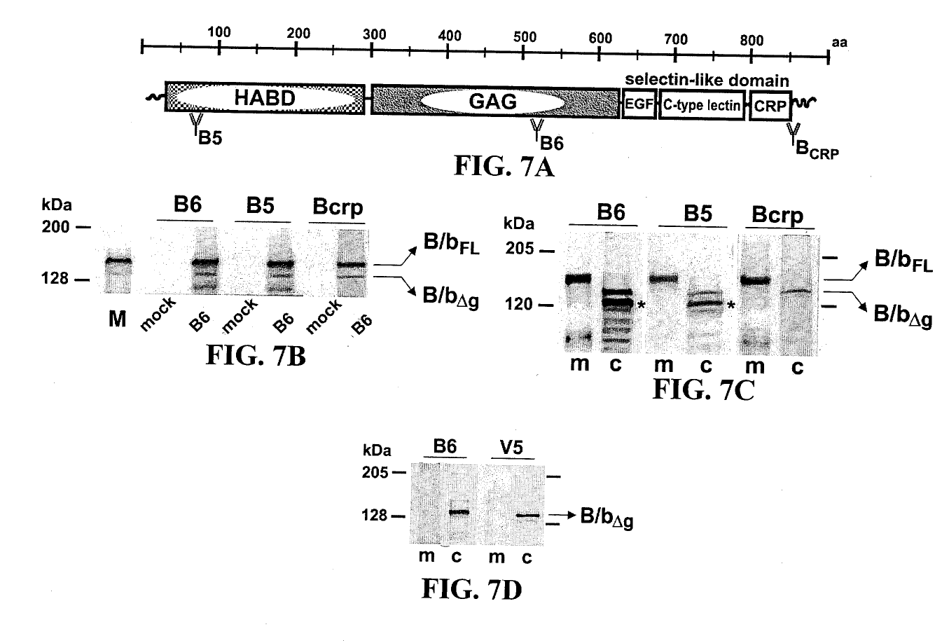

The results described herein demonstrated that glycosylation-variant BEHAB is distinct from the GPI-linked isoform of BEHAB. Another possible source of glycosylation-variant BEHAB is that it is a cleavage product of full-length, full-length BEHAB. The presence of the N- and C-terminal epitopes of full-length BEHAB on glycosylation-variant BEHAB were used to investigate if glycosylation-variant BEHAB is a cleavage product of full-length BEHAB. Full-length BEHAB was immunoprecipitated from a rat brain membrane preparation using the antibody B6, which recognizes an epitope in the GAG-attachment region of rat BEHAB (FIG. 7A). The immunoprecipitated proteins were immunoblotted with antibodies to epitopes located within 10 kDa of the N-(B5) and C-(B_{CRP}) termini of BEHAB (FIG. 7A). If glycosylation-variant BEHAB represents a terminally-truncated cleavage product of full-length BEHAB, one of the N- or C-terminus antibodies would fail to detect the glycosylation-variant BEHAB isoform. However, both full-length BEHAB and glycosylation-variant BEHAB were detected by all three of the anti-BEHAB antibodies (FIG. 7B), suggesting that glycosylation-variant BEHAB was not a cleavage product of full-length BEHAB. Further, detection of glycosylation-variant BEHAB by the C-terminal B_{CRP} antibody provided direct evidence that glycosylation-variant BEHAB was not the GPI-linked isoform, because the CRP domain is absent from the GPI-linked splice variant.

These data demonstrated that glycosylation-variant BEHAB was neither the GPI-linked isoform nor was it a cleavage product of the full-length transcript. To further investigate the molecular source of glycosylation-variant BEHAB, the rat glioma cell line, CNS-1, was transfected with BEHAB cDNA encoding the full-length rat BEHAB protein. Transfected CNS-1 cells produced both full-length BEHAB and the glycosylation-variant BEHAB isoform, and both were detected with all three anti-BEHAB antibodies. Moreover, the distribution of full-length BEHAB and the glycosylation-variant BEHAB isoform closely paralleled that observed in rat brain, with full-length BEHAB found predominantly in the culture medium and glycosylation-variant BEHAB exclusively membrane-associated (FIG. 7C).

BEHAB expression in the mouse oligodendrocyte precursor cell line, Oli-neu, which endogenously expresses both full-length and GPI-anchored BEHAB mRNAs was also studied. Because the antibodies recognized rat BEHAB but not mouse BEHAB, Oli-neu cells were transfected with cDNA encoding the full-length rat BEHAB. In contrast to CNS-1 cells, we detected only the glycosylation-variant isoform in transfected Oli-neu cells. However, like the CNS-1 cells, the expressed glycosylation variant BEHAB isoform was exclusively found in the membrane fraction (FIG. 7D). To confirm these results, Oli-neu cells were transfected with a full-length rat BEHAB cDNA with a C-terminal V5/6xHis epitope. Again, Oli-neu cells only expressed the tagged glycosylation-variant isoform, detected both with anti-BEHAB antibodies and with anti-V5 antibodies (FIG. 7D), providing further evidence that the membrane-associated glycosylation-variant BEHAB isoform represents a full-length BEHAB protein.

### Full-length BEHAB And The Glycosylation-Variant BEHAB Isoform Arise From Differential Glycosylation Of A Single Core Protein

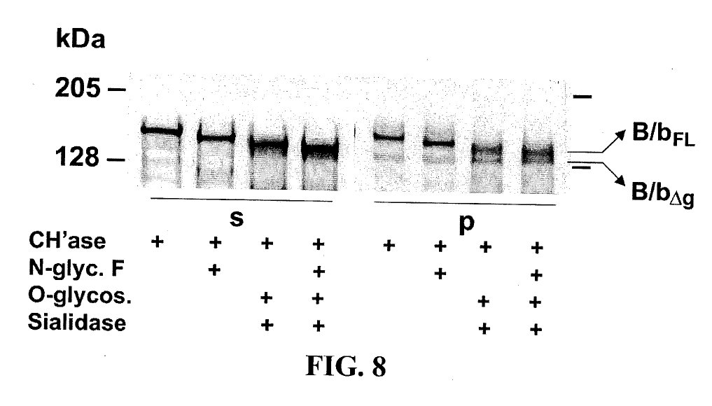

Since glycosylation-variant BEHAB was derived from the same mRNA as full-length BEHAB and was not a result of proteolytic processing, it is possible that a different post-translation modification must be responsible for the size difference between these two isoforms. BEHAB carries N-linked sugars, chondroitin sulfate GAG chains, and additional O-linked sugars. Therefore differences in glycosylation may account for the size difference between glycosylation-variant BEHAB and full-length BEHAB. Unlike full-length BEHAB, the glycosylation-variant BEHAB isoform appeared to lack chondroitin sulfate chains. Chondroitinase ABC treatment neither shifted the apparent molecular mass of the band nor enhanced, or decreased its immunoreactivity. Further, a combination of enzymes that remove N- and O-linked sugars shifted the full-length BEHAB band towards the position of glycosylation-variant BEHAB (FIG. 8). In contrast, the electrophoretic mobility of glycosylation-variant BEHAB was not affected by treatment with glycosidases, indicating that this isoform lacked most of the N- and O-linked sugars present on full-length BEHAB. These results indicated that glycosylation-variant BEHAB is a distinct, un-glycosylated or under-glycosylated isoform of BEHAB/brevican.

### Glycosylation-Variant BEHAB Associates With Brain Membranes By A Calcium-Independent Mechanism

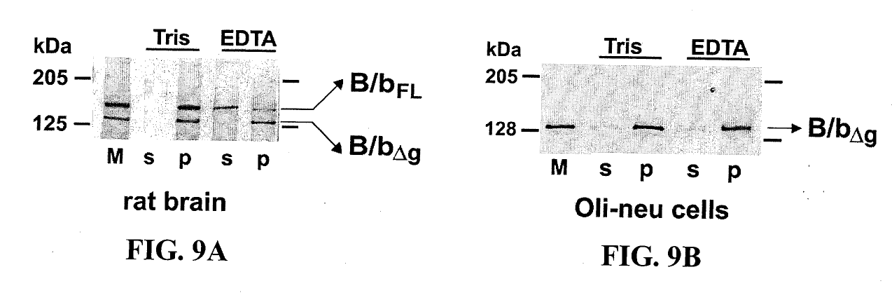

The conditions in which glycosylation-variant BEHAB and the membrane-associated component of full-length BEHAB were released from the membrane preparation were investigated. BEHAB is bound to tenascin-R and a subset of sulfated glycolipids by calcium dependent mechanisms (Asperg et al, 1997, Proc. Natl. Acad. Sci. USA 94: 10116-10121; Miura et al, 1999, J. Biol. Chem. 274: 11431-11438). The calcium-dependent association of BEHAB with the membrane was investigated using the divalent cation chelator, EDTA. Full-length BEHAB was partially released by EDTA, confirming a calcium-dependent association with the membrane. In marked contrast, glycosylation-variant BEHAB was not released by EDTA, indicating association with the membrane by a calcium-independent mechanism. The calcium-independent binding of glycosylation-variant BEHAB to the cell membrane was further supported by V5/6xHis-BEHAB transfected Oli-neu (FIG. 9B) and CNS-1 cells. Glycosylation-variant BEHAB association with the cell membrane in these two cell lines was also EDTA-insensitive, mimicking the behavior of rat brain glycosylation-variant BEHAB.

### Glycosylation-variant BEHAB Is Enriched In The Microsomal Membrane Fraction And Is Present On The Cell Surface

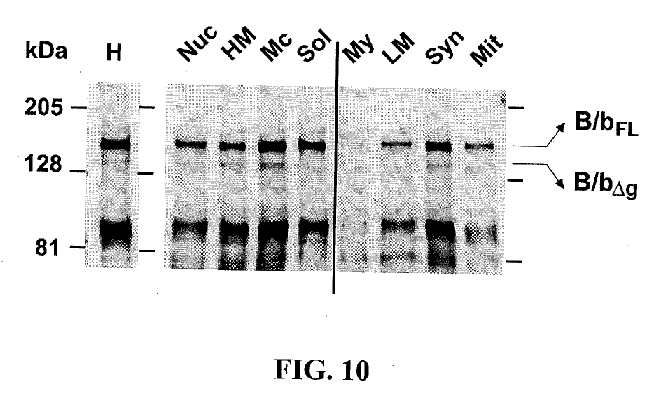

Full-length BEHAB was detected in most of the major subcellular fractions of rat brain, while a glycosylation-variant BEHAB isoform was enriched in the light microsomal fraction (FIG. 10), consistent with an association with the cell membrane. Glycosylation-variant BEHAB was also detected in the heavy mitochondrial/synaptosomal fraction, but a further subfractionation in a discontinuous sucrose gradient, demonstrated that glycosylation-variant BEHAB was restricted to the synaptosomal subfraction, providing further evidence that this isoform likely localizes to the plasma membrane.

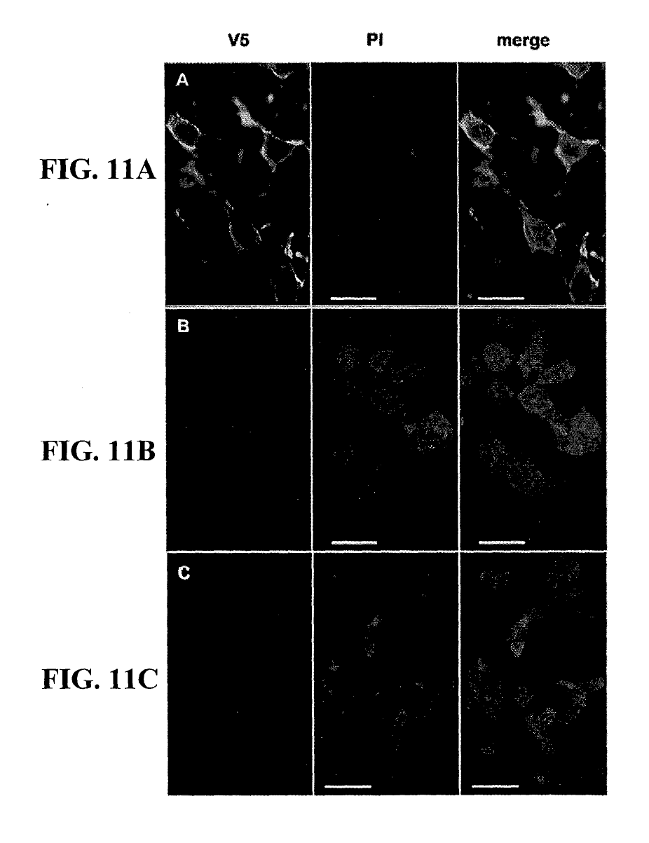

Since the synaptosomal and microsomal fractions contain endoplasmic reticulum membranes, the detection of glycosylation-variant BEHAB in microsomes together with its lack of oligosaccharides raised the possibility that glycosylation-variant BEHAB might be a precursor form of secreted BEHAB, retained in the intracellular secretory pathway due to the lack of post-translational glycosylation. Examination of the subcellular localization of glycosylation-variant BEHAB in Oli-neu cells was used to determine whether glycosylation variant BEHAB was transported to the cell surface. As described herein, Oli-neu cells transfected with BEHAB cDNA expressed only the glycosylation-variant BEHAB isoform (FIG. 7D), thereby providing a model to study its cellular localization in the absence of the full-length BEHAB isoform. Staining of live V5/6xHis-BEHAB transfected Oli-neu cells demonstrated that this isoform was indeed detected on the extracellular surface (FIG. 11). These results demonstrate that, despite its lack of glycosylation, glycosylation-variant BEHAB is transported through the secretory pathway to the cell surface.

### Glycosylation-Variant BEHAB Is The Major Isoform Of BEHAB Upregulated In A Rat Model Of Invasive Glioma

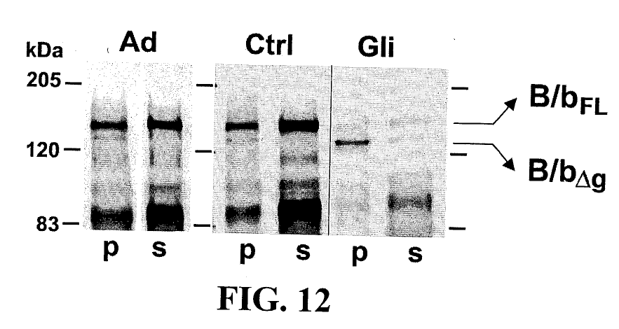

As detailed elsewhere herein, BEHAB mRNA is highly upregulated in human glioma as well as in rat models of invasive glioma. In order to determine if full-length BEHAB and glycosylation-variant BEHAB were differentially regulated in a rat model of invasive glioma, expression of BEHAB isoforms in tumors produced in adult rat brain following stereotaxic placement of CNS-1 cells transfected with BEHAB cDNA was examined. Rat brain glioma tissue was obtained from the hemisphere in which BEHAB cDNA transfected CNS-1 cells were placed, while control tissue was obtained from opposite, untreated hemisphere (FIG. 12). The expression of BEHAB isoforms in the control side was identical to that described above for full-length rat brain (FIG. 5). In the soluble fraction of glioma samples full-length BEHAB expression was reduced compared to control, although these tumors contain much more contaminating blood proteins than normal tissue, making an accurate assessment of soluble proteins difficult due to spurious total protein readings. However, in the particulate fraction of glioma samples glycosylation-variant BEHAB was dramatically upregulated compared to control, and full-length BEHAB was downregulated so that it was almost undetectable compared to control.

### Glycosylation-Variant BEHAB Is Only Detected In Human Glioma Tissues And Not In Healthy Tissue

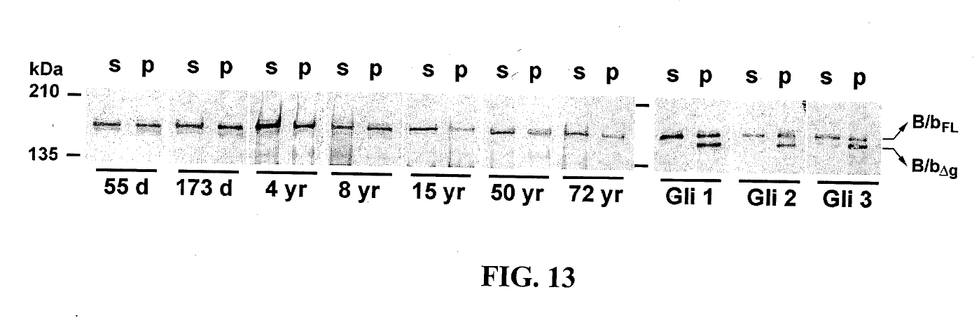

BEHAB mRNA is highly upregulated in human glioma, as well as in rat models of invasive glioma. Glycosylation-variant BEHAB represents the major upregulated BEHAB isoform in a rat model of glioma and also in surgical samples of human glioma. In order to determine whether full-length BEHAB and glycosylation-variant BEHAB were differentially regulated in human gliomas, the expression of BEHAB isoforms in surgical samples of human gliomas was compared to post-mortem normal human brain samples. Brain homogenates were prepared essentially as described elsewhere herein. In normal human brain samples, a full-length BEHAB isoform was detected with an approximate molecular weight of 160 kDa (after treatment with chondroitinase) at all ages examined, from 55 days old to 72 years old (FIG. 13). It is important to note that human full-length BEHAB runs 10 kDa larger than rat full-length BEHAB, which is due in part to the fact that the primary amino acid sequence of human BEHAB is longer than that of rat. In the soluble fraction from human glioma samples, BEHAB expression was not detectably increased over normal levels. The marked increase in expression of BEHAB mRNA in glioma (Jaworski et al., 1996, Cancer Research, 56: 2293-2298; Gary et al., 2000, Gene 256: 139-147) likely gives rise instead to the dramatic upregulation of glycosylation-variant BEHAB, which is detected only in the particulate (membrane) fraction from glioma samples (FIG. 13). While rat glycosylation-variant BEHAB is 20 kDa smaller than rat full length BEHAB, human glycosylation-variant BEHAB is only about 10 kDa smaller than the human full length BEHAB. In both rat and human, glycosylation-variant BEHAB is only found in the membrane fraction. However, adult human glycosylation-variant BEHAB is only detected in glioma and not in normal brain, while both isoforms are detected in the normal rat brain. BEHAB mRNA and protein expression are down regulated in the adult human brain (Gary et al., 2000, Gene 256: 139-147), while expression levels remain high into adulthood in the rat. In assays of more than 15 samples of human brain from various ages, glycosylation-variant BEHAB has not been detected in normal human brain, but human glycosylation-variant BEHAB was detected in every glioma sample assayed. These data indicate that glycosylation-variant BEHAB represents a glioma-specific isoform and is the major upregulated isoform of BEHAB in glioma.

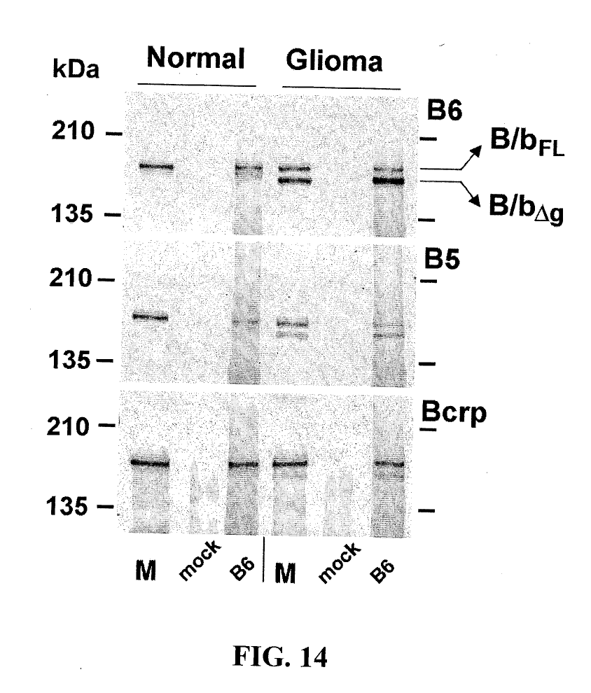

The human glycosylation-variant BEHAB isoform, as in the rat, is not a cleavage product of full-length BEHAB. The N- and C-terminal epitopes of full-length BEHAB were also detected on glycosylation-variant BEHAB. Full-length BEHAB was immunoprecipitated from normal human brain and from glioma membrane preparations using the antibody B6, which recognizes an epitope in the GAG-attachment region of human BEHAB (FIG. 7). The immunoprecipitated proteins were immunoblotted with antibodies to epitopes located within 10 kDa of the N-(B5) and C-(B_{CRP}) termini of BEHAB (FIG. 7A). If glycosylation-variant BEHAB represented a terminally-truncated cleavage product of full length BEHAB, one of the N- or C-terminus antibodies would fail to detect a glycosylation-variant BEHAB isoform found in glioma. However, as demonstrated in experiments with rat glycosylation-variant BEHAB, both human full-length BEHAB and human glycosylation-variant BEHAB were detected by all three of the anti-BEHAB antibodies (FIG. 14), demonstrating that glycosylation-variant BEHAB is not a cleavage product of full-length BEHAB. Further, detection of glycosylation-variant BEHAB in glioma samples by the C-terminal B_{CRP} antibody provided direct evidence that glycosylation-variant BEHAB was not the GPI-linked BEHAB isoform, because the CRP domain is absent from the GPI-linked splice variant.

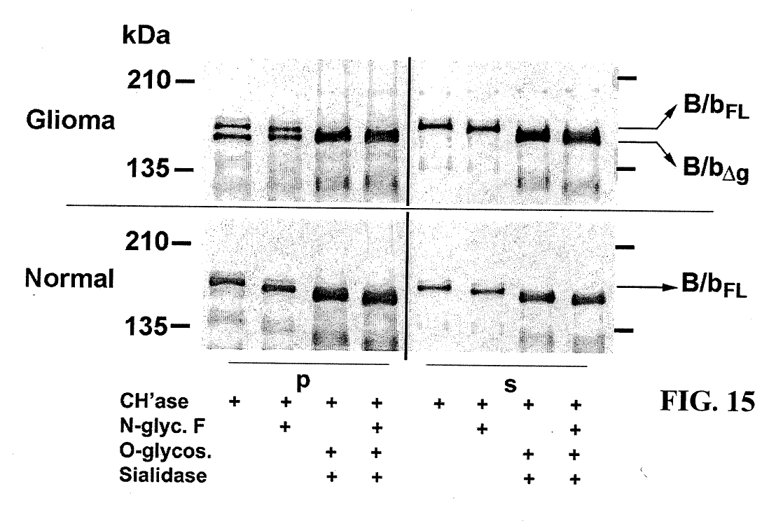

Since glycosylation-variant BEHAB was derived from the same mRNA as full-length BEHAB and was not a result of proteolytic processing, a different post-translation modification may be responsible for the size difference between these two isoforms. In addition, while rat glycosylation-variant BEHAB is 20 kDa smaller than rat full-length BEHAB, human glycosylation-variant BEHAB is only 10 kDa smaller than full-length human BEHAB (FIGS. 5 and 13), suggesting species differences in post-translation modification. BEHAB in both rat and human carries N-linked sugars, chondroitin sulfate GAG chains, and additional O-linked sugars. Therefore, differences in glycosylation may account for the size difference between glycosylation-variant BEHAB and full-length BEHAB and between BEHAB from rat and human. Unlike full-length BEHAB, glycosylation-variant BEHAB appeared to lack chondroitin sulfate chains. Chondroitinase ABC treatment neither shifted the apparent molecular mass of the band nor enhanced, or decreased its immunoreactivity (FIG. 15). Further, a combination of enzymes that removed N- and O-linked sugars shifted the full-length BEHAB band towards the position of glycosylation-variant BEHAB in both normal brain and glioma (FIG. 15). In contrast, the electrophoretic mobility of glycosylation-variant BEHAB in glioma was not affected by treatment with glycosidases, indicating that glycosylation-variant BEHAB lacked most, if not all, of the N- and O-linked sugars present on full-length BEHAB. These results indicated that glycosylation-variant BEHAB is a distinct, un-glycosylated or under-glycosylated isoform of BEHAB. Interestingly, in the rat brain, full-length BEHAB runs at an approximate molecular weight of 150 kDa and shifts to an approximate molecular weight of 130 kDa when deglycosylated (FIG. 8). In contrast, full-length human BEHAB runs at an approximate molecular weight of 160 kDa but is shifted by only 10 kDa to 150 kDa by enzymatic deglycosylation (FIG. 15). This indicates that species differences in glycosylation account for the difference in sizes of these isoforms in rats and humans. Further, these results suggest that despite these size differences, rat and human glycosylation-variant BEHAB appear to be derived by a common mechanism and are biochemically similar molecules.

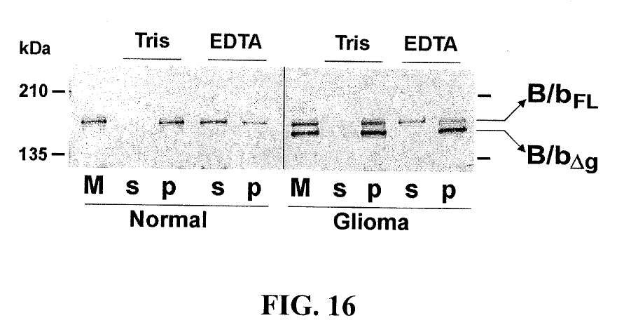

The conditions in which human glycosylation-variant BEHAB and the membrane-associated component of full-length BEHAB were released from normal human brain and glioma membrane preparations were investigated. BEHAB is bound to tenascin-R and a subset of sulfated glycolipids by calcium dependent mechanisms (Asperg et al, 1997, Proc. Natl. Acad. Sci. USA 94: 10116-10121; Miura et al, 1999, J. Biol. Chem. 274: 11431-11438). The calcium-dependent association of BEHAB with the membrane was investigated using the divalent cation chelator, EDTA. Full-length BEHAB was partially released by EDTA in samples from both normal human brain and glioma, confirming a calcium-dependent association with the membrane as was demonstrated in rat. In marked contrast, glycosylation-variant BEHAB was not released by EDTA in glioma samples, indicating association with the membrane by a calcium-independent mechanism (FIG. 16). Glycosylation-variant BEHAB in human glioma mimics the behavior of rat brain glycosylation-variant BEHAB, suggesting that glycosylation-variant BEHAB from rat and human are binding to the cell membrane by molecularly similar, or identical, mechanisms.

### Discussion

Previous work has demonstrated that the 9L gliosarcoma cell line, which grows as a non-invasive tumor, becomes invasive when transfected with a 5′ fragment of BEHAB (Zhang et al., 1998, J. Neuroscience 18: 2370-2376). However, transfection of 9L cells with full-length BEHAB does not change the tumor phenotype from wild type 9L cells. In sharp contrast, CNS-1 cells transfected with full-length BEHAB increase the size and invasiveness of tumors. The difference in phenotype of the different cell lines is explained by differences in the ability of these two cell lines to cleave BEHAB. CNS-1 cells cleave the full-length protein at the Glu³⁹⁵-Ser³⁹⁶ cleavage site, creating 90 kDa and 50 kDa fragments. In contrast, 9L cells do not proteolytically process the full-length protein. Additionally, previous studies showed that BEHAB cleavage in the CNS-1 cells is mediated by ADAMTS-4 (Matthews et al., 2000, J. Biol. Chem. 275: 22695-22703). The role of BEHAB cleavage in glioma progression was evaluated using CNS-1 cells transfected with a mutated, uncleavable, form of BEHAB. The effects of this construct on tumor progression and animal survival were studied, and for the first time firmly demonstrate the role of BEHAB cleavage in the progression of gliomas.

Mutation of BEHAB/brevican cleavage site from ³⁹³Glu-Ser-Glu-Ser-Arg-Gly³⁹⁸ to ³⁹³Glu-Ser-Glu-Asn-Val-Tyr³⁹⁸ (SEQ ID NO:1 and SEQ ID NO:2, respectively) made the protein completely uncleavable by CNS-1 cells. Importantly, this uncleavable mutant had a different effect than the normal full-length construct on rat CNS-1 tumors. While CNS-1-FL tumors were larger and decreased the survival time of animals relative to animals with control tumors, CNS-1-NVY tumors were not phenotypically distinguishable from the control tumors. These studies clearly demonstrate the critical role that both cleavage and processing play in BEHAB function in primary CNS tumors.

CNS-1-NVY tumors were smaller than CNS-1-FL tumors, but phenotypically similar to control tumors. This result is informative about the mechanism of BEHAB cleavage-mediated effects in gliomas, especially in light of the fact that CNS-1 cells are induced to express endogenous BEHAB when grown in the brain. Accordingly, all three of the cell lines used in these experiments express normal levels of endogenous BEHAB; CNS-1-FL tumors express, in addition to endogenous BEHAB, exogenous BEHAB in a cleavable form; and CNS-1-NVY tumors express, in addition to endogenous BEHAB, exogenous BEHAB in an uncleavable form. Therefore, if the NVY mutant was working as a dominant negative in CNS-1 cell tumors, it would be expected that the CNS-1-NVY tumors would be significantly smaller than the control tumors (CNS-1-GFP) by disrupting the normal function of the BEHAB, which is not the case. Rather, it appears that the CNS-1-NVY tumors have instead have been transfected with a molecule that produces no change in their phenotype compared to control tumors, strongly suggesting that the cleavage products of BEHAB mediate unique interactions and/or functions not mediated by the full-length protein. As an example, if BEHAB cleavage products simply solublized the matrix and allowed cell movement, the production of uncleavable BEHAB would counteract this effect. However, tumors with or without the uncleavable substrate seem to be very similar. These results suggest that BEHAB cleavage products have a unique function that is not mediated by the full-length protein itself. The data evident herein demonstrate that BEHAB cleavage potentiates the progression of primary CNS tumors and that inhibiting BEHAB cleavage can reduce tumor progression. These studies strongly indicate that inhibition of BEHAB cleavage may represent an important new therapeutic strategy, and therefore inhibition of BEHAB cleavage or the function of the cleavage products will serve as an effective and novel method for treating primary CNS tumors.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

We claim:

1. An isolated rat glycosylation-variant BEHAB isoform, wherein the glycosylation-variant BEHAB isoform has a molecular weight of about 130 kDa.

2. The glycosylation-variant BEHAB isoform of claim 1, wherein the isolated nucleic acid encoding the glycosylation-variant BEHAB isoform comprises the isolated nucleic acid of SEQ ID NO: 5.

3. An isolated rat underglycosylated BEHAB isoform, wherein the underglycosylated BEHAB isoform has a molecular weight of about 130 kDa.

4. The underglycosylated BEHAB isoform of claim 3, wherein the isolated nucleic acid encoding the underglycosylated BEHAB isoform comprises the isolated nucleic acid of SEQ ID NO: 5.

5. An isolated rat unglycosylated BEHAB isoform, wherein the unglycosylated BEHAB isoform has a molecular weight of about 130 kDa.

6. The unglycosylated BEHAB isoform of claim 5, wherein the isolated nucleic acid encoding the unglycosylated BEHAB isoform comprises the isolated nucleic acid of SEQ ID NO: 5.

7. An isolated human glycosylation-variant BEHAB isoform, wherein the glycosylation-variant BEHAB isoform has a molecular weight of about 150 kDa.

8. The glycosylation-variant BEHAB isoform of claim 7, wherein the isolated nucleic acid encoding the glycosylation-variant BEHAB isoform comprises the isolated nucleic acid of SEQ ID NO: 7.

9. An isolated human underglycosylated BEHAB isoform, wherein the underglycosylated BEHAB isoform has a molecular weight of about 150 kDa.

10. The underglycosylated BEHAB isoform of claim 9, wherein the isolated nucleic acid encoding the underglycosylated BEHAB isoform comprises the isolated nucleic acid of SEQ ID NO: 7.

11. An isolated human unglycosylated BEHAB isoform, wherein the unglycosylated BEHAB isoform has a molecular weight of about 150 kDa.

12. The unglycosylated BEHAB isoform of claim 11, wherein the isolated nucleic acid encoding the unglycosylated BEHAB isoform comprises the isolated nucleic acid of SEQ ID NO: 7.

13. A method of making a glycosylation-variant BEHAB isoform, the method comprising transfecting a cell with an isolated nucleic acid encoding a BEHAB protein and isolating a glycosylation-variant BEHAB therefrom.

14. The method of claim 13, wherein the cell is an Oli-neu cell.

15. The method of claim 13, wherein the isolated nucleic acid encoding BEHAB protein comprises the isolated nucleic acid of SEQ ID NO:5.

16. A method of detecting a glycosylation-variant BEHAB isoform in a mammal, the method comprising contacting a biological sample of the mammal with an antibody that specifically binds with a glycosylation-variant BEHAB isoform or fragment thereof, and detecting the binding of the antibody to the biological sample, wherein binding of the antibody with the sample detects a glycosylation-variant BEHAB isoform in a mammal.

17. The method of claim 16, wherein the mammal is a human.

18. The method of claim 16, wherein the antibody is selected from the group consisting of B5, B6, or B_{CRP}.

19. The method of claim 16, wherein the antibody comprises a tag polypeptide covalently linked thereto.

20. A method of diagnosing a primary CNS tumor in a mammal, the method comprising obtaining a biological sample from a mammal suspected of having a primary CNS tumor, assessing the level of a glycosylation-variant BEHAB isoform in the biological sample, and comparing the level of a glycosylation-variant BEHAB isoform in the biological sample with the level of a glycosylation-variant BEHAB isoform in a biological sample obtained from a mammal not suspected of having a primary CNS tumor, wherein a higher level of a glycosylation-variant BEHAB isoform in the biological sample from the mammal suspected of having a primary CNS tumor compared with the level of a glycosylation-variant BEHAB isoform in the biological sample from the mammal not suspected of having a primary CNS tumor is an indication that the mammal suspected of having a primary CNS tumor has a primary CNS tumor, thereby diagnosing a primary CNS tumor in a mammal.

21. The method of claim 20, wherein the mammal is a human.

22. The method of claim 20, wherein the biological sample is selected from the group consisting of blood, neural tissue, cerebrospinal fluid, urine, saliva and brain tissue.

23. A method of treating a primary CNS tumor in a mammal, the method comprising administering to a mammal an effective amount of a glycosylation-variant BEHAB isoform inhibitor, thereby treating a primary CNS tumor in a mammal.

24. The method of claim 23, wherein the mammal is a human.

25. The method of claim 23, wherein the glycosylation-variant BEHAB isoform inhibitor is selected from the group consisting of an antibody, a protein and a peptidomimetic.

26. The method of claim 25, wherein the antibody specifically binds to a glycosylation-variant BEHAB isoform, or fragment thereof.

27. A method of assessing the effectiveness of a treatment for a primary CNS tumor in a mammal, the method comprising assessing the level of a glycosylation-variant BEHAB isoform in the mammal before, during, or after administration of a treatment for a primary CNS tumor to said mammal, wherein a lower level of the glycosylation-variant BEHAB isoform in the mammal during or after administration of the treatment for a primary CNS tumor with the level of the glycosylation-variant BEHAB isoform in the mammal before administration of the treatment for a primary CNS tumor is an indication of the effectiveness of the treatment for a primary CNS tumor in the mammal, thereby assessing the effectiveness of the treatment for a primary CNS tumor in the mammal.

28. The method of claim 27, wherein the mammal is a human.

29. The method of claim 28, wherein the treatment for a primary CNS tumor is selected from the group consisting of chemotherapy, radiation therapy, and surgery.

30. A method of identifying a compound that affects expression of a glycosylation-variant BEHAB isoform in a cell, the method comprising contacting a cell with a test compound and comparing the level of glycosylation-variant BEHAB isoform expression in the cell with the level of glycosylation-variant BEHAB isoform expression in an otherwise identical cell not contacted with the test compound, wherein a higher or lower level of glycosylation-variant BEHAB isoform expression in the cell contacted with the test compound compared with the level of glycosylation-variant BEHAB isoform expression in the otherwise identical cell not contacted with the test compound is an indication that the test compound affects expression of the glycosylation-variant BEHAB isoform in a cell, thereby identifying a compound that affects expression of the glycosylation-variant BEHAB isoform in a cell.

31. A compound identified by the method of claim 30.

32. A method of identifying a compound that reduces expression of a glycosylation-variant BEHAB isoform in a cell, the method comprising contacting a cell with a test compound and comparing the level of glycosylation-variant BEHAB isoform expression in the cell with the level of glycosylation-variant BEHAB isoform expression in an otherwise identical cell not contacted with the test compound, wherein a higher or lower level of glycosylation-variant BEHAB isoform expression in the cell contacted with the test compound compared with the level of glycosylation-variant BEHAB isoform expression in the otherwise identical cell not contacted with the test compound is an indication that the test compound reduces expression of the glycosylation-variant BEHAB isoform in a cell, thereby identifying a compound that reduces expression of the glycosylation-variant BEHAB isoform in a cell.

33. A compound identified by the method of claim 32.

34. A method of treating a primary CNS tumor in a mammal, the method comprising isolating a cell from a mammal, contacting the cell with a glycosylation-variant BEHAB isoform, or a fragment thereof, and administering the cell so contacted to the mammal.

35. The method of claim 34, wherein the cell is an antigen presenting cell.

36. The method of claim 34, wherein the cell is a dendritic cell.

37. A kit for detecting a glycosylation-variant BEHAB isoform, the kit comprising an antibody to a glycosylation-variant BEHAB isoform, the kit further comprising an instructional material for the use thereof.

38. A kit for diagnosing a primary CNS tumor in a mammal, the kit comprising an antibody to a glycosylation-variant BEHAB isoform, the kit further comprising an applicator, and an instructional material for the use thereof.

39. A kit for treating a primary CNS tumor, the kit comprising a composition comprising an antibody that specifically binds with a glycosylation-variant BEHAB isoform, or a fragment thereof, and a pharmaceutically acceptable carrier, the kit further comprising an applicator, and an instructional material for use thereof.

40. A kit for treating a primary CNS tumor with immune therapy, the kit comprising a glycosylation-variant BEHAB isoform, or a fragment thereof, the kit further comprising an applicator, and an instructional material for use thereof.
